(19) United States
(12) Patent Application Publication
(10) Pub. No.: US 2024/0010745 A1
(43) Pub. Date: Jan. 11, 2024

(54) METHODS AND COMPOSITIONS FOR DEPLETING NATURAL KILLER CELLS AND USES THEREOF IN CELLULAR THERAPIES

(71) Applicant: Nanjing Legend Biotech Co., Ltd., Nanjing (CN)

(72) Inventors: Ming Zeng, Irving, TX (US); Huihui Zhang, Nanjing (CN)

(21) Appl. No.: 18/038,623

(22) PCT Filed: Dec. 14, 2021
(86) PCT No.: PCT/CN2021/137876

§ 371 Date: May 24, 2023

(30) Foreign Application Priority Data
Dec. 14, 2020 (WO) PCT/CN2020/136157

(51) Int. Cl. C07K 16/28; A61K 39/00; C07K 16/08; A61P 35/00

(52) U.S. Cl. CPC C07K 16/2896; A61K 39/4611; A61K 39/4622; A61K 39/4631; C07K 16/2878; C07K 16/087; C07K 16/2803; C07K 16/2851; C07K 16/283; C07K 16/2866; C07K 16/2875; A61P 35/00; A61K 39/464411; A61K 39/464838; A61K 39/464419; A61K 39/464429; A61K 39/464417; A61K 2039/505; C07K 2317/732; C07K 2317/734; C07K 2317/21; C07K 2317/56; C07K 2317/569; A61K 2239/17; A61K 2239/21; A61K 2239/22; A61K 2239/48; C07K 2317/73; C07K 2317/53; C07K 2319/03; C07K 2319/02

(57) ABSTRACT

An engineered immune cell comprising a first functional exogenous receptor capable of binding and depleting natural killer (NK) cells, and a second functional exogenous receptor, wherein the engineered immune cell has reduced MHC I on cell surface.

### CROSS REFERENCE TO RELATED APPLICATIONS

This application claims benefit of priority of the International Application No. PCT/CN2020/136157 filed on Dec. 14, 2020, the content of which is incorporated herein by reference in its entirety.

### SEQUENCE LISTING

This application incorporates by reference a Sequence Listing submitted with this application in a text format, entitled “L2-W20245WO_SEQ_LISTING,” created on Dec. 10, 2021 having a size of 65,301 bytes.

### 1. FIELD

Provided herein are methods or compositions for depleting host natural killer cells in connection with cellular therapies (e.g., allogeneic CAR-T cell therapies) for treating a disease or disorder such as cancer.

### 2. BACKGROUND

Adoptive transfer of T cells represents an emerging innovative therapeutic strategy against cancer. Allogeneic T cell therapies are of particular interest due to their potential for providing cost and time efficient treatments to patients. However, in the field of allogeneic cellular therapies, e.g., using healthy donor cells, two important issues need to be addressed. The first issue is graft-versus-host disease (GVHD), which is caused by donor-derived T cells that recognize HLA mismatches through T-cell receptors (TCRs) and attack patient tissues. It can be fatal even in the case of HLA-matched donors, as minor mismatches can still cause a reaction. It can also occur after blood transfusion. But due to widespread albinism of blood products and radiation on high-risk populations (usually those who have been severely immunosuppressed after purine-like chemotherapy), it is rarely seen now. The second issue is that allogeneic T cells are recognized as foreign bodies and rejected by the recipient's immune system because they are not HLA-matched. Therefore, there is still a need in the art for improved methods and engineered immune cells particularly for allogeneic therapies for treating a disease or disorder such as cancer.

### 3. SUMMARY

In one aspect, provided herein is a method for treating a disease or disorder in a subject comprising administering to the subject an engineered immune cell, wherein the method comprises depleting the subject's immune cells (such as natural killer (NK) cells), and wherein the engineered immune cell has a reduced expression of the MHC I molecule on its surface.

In some embodiments, provided herein is a method for treating a disease or disorder in a subject comprising: (i) administering to the subject an agent comprising an antibody capable of binding an antigen expressed on immune cells (such as NK cells), thereby depleting the immune cells; and (ii) administering to the subject an engineered immune cell (such as an engineered T cell) comprising a functional exogenous receptor after step (i), wherein the functional exogenous receptor comprises an extracellular binding domain, a transmembrane domain, and an intracellular signaling domain, wherein the engineered immune cell (such as an engineered T cell) has reduced MHC I on cell surface, and wherein optionally the endogenous expression of the antigen targeted by the antibody is down regulated in the engineered immune cell (such as an engineered T cell).

In some embodiments, the antigen targeted by the antibody is also a cell surface marker on an activated T cell.

In some embodiments, the antibody is capable of depleting NK cells via antibody-dependent cellular cytotoxicity (ADCC), complement-dependent cytotoxicity (CDC), antibody-dependent cellular phagocytosis (ADCP), or induced apoptosis. In other embodiments, the agent comprising the antibody is an antibody-drug conjugate (ADC) and the drug conjugated to the antibody is capable of depleting NK cells.

In some embodiments, the expression of the MHC I is reduced by expressing MHC I knockdown molecule on the engineered immune cell. In some embodiments, the MHC I knockdown molecule is sICP47 protein. In some embodiments, the sICP47 protein is derived from ICP47 of Simian Agent 8 (SA8), Cercopithecine herpesvirus 16 (CeHV-16), Cercopithecine herpesvirus 1 (CeHV-1), Macacine alphaherpesvirus 1, Pappine alphaherpesvirus 2 or functional variant thereof. In some embodiments, the sICP47 protein comprises an amino acid sequence selected from the group consisting of SEQ ID NOs: 10-15 or variants thereof.

In some embodiments, the antibody is an anti-CD38 antibody, wherein optionally (i) the anti-CD38 antibody comprises a VH comprising an amino acid sequence of SEQ ID NO: 1, and/or a VL comprising an amino acid sequence of SEQ ID NO: 2; or (ii) the anti-CD38 antibody comprises a VH comprising an amino acid sequence of SEQ ID NO: 3, and/or a VL comprising an amino acid sequence of SEQ ID NO: 4.

In some embodiments, the antibody is an anti-CS1 antibody, wherein optionally the anti-CS1 antibody comprises a VH comprising an amino acid sequence of SEQ ID NO: 5, and/or a VL comprising an amino acid sequence of SEQ ID NO: 6.

In some embodiments, the antibody is an anti-IL-T2 antibody, wherein optionally the anti-IL-T2 antibody comprises a VH comprising an amino acid sequence of SEQ ID NO: 28, and/or a VL comprising an amino acid sequence of SEQ ID NO: 29.

In some embodiments, the antibody is an anti-CD137 antibody, wherein optionally the anti-CD137 antibody comprises a VH comprising an amino acid sequence of SEQ ID NO: 30, and/or a VL comprising an amino acid sequence of SEQ ID NO: 31.

In some embodiments, the antibody is an anti-NKG2A antibody, wherein optionally the anti-NKG2A antibody comprises a VH comprising an amino acid sequence of SEQ ID NO: 32, and/or a VL comprising an amino acid sequence of SEQ ID NO: 33.

In some embodiments, the antibody is an anti-NKG2D antibody, wherein optionally (i) the anti-NKG2D antibody comprises a VH comprising an amino acid sequence of SEQ ID NO: 34, and/or a VL comprising an amino acid sequence of SEQ ID NO: 35; or (ii) the anti-NKG2D antibody comprises a VH comprising an amino acid sequence of SEQ ID NO: 36, and/or a VL comprising an amino acid sequence of SEQ ID NO: 37.

In some embodiments, the antibody is an anti-CD16 antibody, wherein optionally the anti-CD16 antibody comprises a VH comprising an amino acid sequence of SEQ ID NO: 38, and/or a VL comprising an amino acid sequence of SEQ ID NO: 39.

In some embodiments, the antibody is an anti-CD16a antibody, wherein optionally the anti-CD16a antibody comprises a VH comprising an amino acid sequence of SEQ ID NO: 40, and/or a VL comprising an amino acid sequence of SEQ ID NO: 41.

In some embodiments, the antibody is an anti-KIR antibody, wherein optionally the anti-KIR antibody comprises a VH comprising an amino acid sequence of SEQ ID NO: 42, and/or a VL comprising an amino acid sequence of SEQ ID NO: 43.

In some embodiments, the antibody is an anti-CD56 antibody, wherein optionally the anti-CD56 antibody comprises a VH comprising an amino acid sequence of SEQ ID NO: 44, and/or a VL comprising an amino acid sequence of SEQ ID NO: 45.

In some embodiments, the antibody is an anti-CD226 antibody, wherein optionally (i) the anti-CD226 antibody comprises a VH comprising an amino acid sequence of SEQ ID NO: 46, and/or a VL comprising an amino acid sequence of SEQ ID NO: 47; or (ii) the anti-CD226 antibody comprises a VH comprising an amino acid sequence of SEQ ID NO: 48, and/or a VL comprising an amino acid sequence of SEQ ID NO: 49.

In some embodiments, the antibody is an anti-CD25 antibody, wherein optionally (i) the anti-CD25 antibody comprises a VH comprising an amino acid sequence of SEQ ID NO: 50, and/or a VL comprising an amino acid sequence of SEQ ID NO: 51; or (ii) the anti-CD226 antibody comprises a VH comprising an amino acid sequence of SEQ ID NO: 52, and/or a VL comprising an amino acid sequence of SEQ ID NO: 53.

In some embodiments, the antibody is an anti-CD83 antibody, wherein optionally the anti-CD83 antibody comprises a VH comprising an amino acid sequence of SEQ ID NO: 54, and/or a VL comprising an amino acid sequence of SEQ ID NO: 55.

In some embodiments, the antibody is an anti-KLRG1 antibody, wherein optionally the anti-KLRG1 antibody comprises a VH comprising an amino acid sequence of SEQ ID NO: 56, and/or a VL comprising an amino acid sequence of SEQ ID NO: 57.

In some embodiments, the antibody is an anti-CD70 antibody, wherein optionally the anti-CD70 antibody comprises a VH comprising an amino acid sequence of SEQ ID NO: 58, and/or a VL comprising an amino acid sequence of SEQ ID NO: 59.

In some embodiments, the antibody is an anti-CD30 antibody, wherein optionally the anti-CD30 antibody comprises a VH comprising an amino acid sequence of SEQ ID NO: 60, and/or a VL comprising an amino acid sequence of SEQ ID NO: 61.

In some embodiments, the antibody is an anti-CD229 antibody, wherein optionally the anti-CD229 antibody comprises a VH comprising an amino acid sequence of SEQ ID NO: 62, and/or a VL comprising an amino acid sequence of SEQ ID NO: 63.

In some embodiments, the antibody is an anti-NCR3 antibody, wherein optionally the anti-NCR3 antibody comprises a VH comprising an amino acid sequence of SEQ ID NO: 64, and/or a VL comprising an amino acid sequence of SEQ ID NO: 65.

In some embodiments, the antibody is a monospecific or multispecific antibody.

In some embodiments, the functional exogenous receptor is a T cell receptor (TCR), a chimeric antigen receptor (CAR), a chimeric TCR (cTCR), or a T cell antigen coupler (TAC)-like chimeric receptor.

In some more specific embodiments, the functional exogenous receptor is a CAR In some embodiments, the extracellular binding domain of the CAR comprises an antigen binding domain capable of binding a tumor antigen. In some embodiments, the tumor antigen is BCMA. In some embodiments, the antigen binding domain comprises an amino acid sequence of SEQ ID NO: 7, SEQ ID NO: 8 and/or SEQ ID NO: 9.

In another aspect, provided herein is an engineered immune cell (such as an engineered T cell) comprising (i) a first functional exogenous receptor capable of binding and depleting natural killer (NK) cells, comprising a first extracellular binding domain capable of binding to a first antigen, a first transmembrane domain, and a first intracellular signaling domain; and (ii) a second functional exogenous receptor comprising a second extracellular binding domain capable of binding to a second antigen, a second transmembrane domain, and a second intracellular signaling domain, wherein the engineered immune cell (such as an engineered T cell) has reduced MHC I on cell surface, and wherein optionally the endogenous expression of the first antigen is down regulated in the engineered immune cell (such as an engineered T cell). In some embodiments, the first antigen is also a cell surface marker on an activated T cell.

In some embodiments, the expression of the MHC I is reduced by expressing MHC I knockdown molecule on the engineered immune cell. In some embodiments, the MHC I knockdown molecule is sICP47 protein. In some embodiments, the sICP47 protein is derived from ICP47 of Simian Agent 8 (SA8), Cercopithecine herpesvirus 16 (CeHV-16), Cercopithecine herpesvirus 1 (CeHV-1), Macacine alphaherpesvirus 1, Pappine alphaherpesvirus 2 or functional variant thereof. In some embodiments, the sICP47 protein comprises an amino acid sequence selected from the group consisting of SEQ ID NOs: 10-15 or variants thereof.

In some embodiments, the first functional exogenous receptor is a first chimeric antigen receptor (CAR), and wherein the second functional exogenous receptor is a T cell receptor (TCR), a chimeric TCR (cTCR), a T cell antigen coupler (TAC)-like chimeric receptor or a second CAR. In some embodiments, the second functional exogenous receptor is a second CAR.

In some embodiments, the first antigen is selected from a group consisting of CD38, CS1, IL-T2, CD137, NKG2A, NKG2D, CD16, CD56, CD138, CD25, CD69, SLAM family members, L-SELECTIN, CD226, Kir family members, TIGIT, TRAIL, and the natural cytotoxicity receptors NCR1, NCR2, NCR3, wherein optionally SLAM family members are selected from a group consisting of SLAMF1, SLAMF4(2B4), SLAMF6, SLAMF3 and SLAMF5, and wherein optionally Kir family members are selected from a group consisting of KIR2DL1, KIR2DS1, KIR2DL2/L3, KIR2DS2, KIR2DS4, KIR3DL1, and KIR3DL2.

In some embodiments, the first extracellular binding domain comprises:
(i) an anti-CD38 binding domain, wherein optionally (i) the anti-CD38 binding domain comprises a VH comprising an amino acid sequence of SEQ ID NO: 1, and/or a VL comprising an amino acid sequence of SEQ ID NO: 2; or (ii) the anti-CD38 binding domain comprises a VH comprising an amino acid sequence of SEQ ID NO: 3, and/or a VL comprising an amino acid sequence of SEQ ID NO: 4;
(ii) an anti-CS1 binding domain, wherein optionally the anti-CS1 binding domain comprises a VH comprising an amino acid sequence of SEQ ID NO: 5, and/or a VL comprising an amino acid sequence of SEQ ID NO: 6;
(iii) an anti-IL-T2 binding domain, wherein optionally the anti-IL-T2 binding domain comprises a VH comprising an amino acid sequence of SEQ ID NO: 28, and/or a VL comprising an amino acid sequence of SEQ ID NO: 29;
(iv) an anti-CD137 binding domain, wherein optionally the anti-CD137 binding domain comprises a VH comprising an amino acid sequence of SEQ ID NO: 30, and/or a VL comprising an amino acid sequence of SEQ ID NO: 31;
(v) an anti-NKG2A binding domain, wherein optionally the anti-NKG2A antibody comprises a VH comprising an amino acid sequence of SEQ ID NO: 32, and/or a VL comprising an amino acid sequence of SEQ ID NO: 33;
(vi) an anti-NKG2D binding domain, wherein optionally (i) the anti-NKG2D binding domain comprises a VH comprising an amino acid sequence of SEQ ID NO: 34, and/or a VL comprising an amino acid sequence of SEQ ID NO: 35; or (ii) the anti-NKG2D binding domain comprises a VH comprising an amino acid sequence of SEQ ID NO: 36, and/or a VL comprising an amino acid sequence of SEQ ID NO: 37;
(vii) an anti-CD16 binding domain, wherein optionally the anti-CD16 binding domain comprises a VH comprising an amino acid sequence of SEQ ID NO: 38, and/or a VL comprising an amino acid sequence of SEQ ID NO: 39;
(viii) an anti-CD16a binding domain, wherein optionally the anti-CD16a binding domain comprises a VH comprising an amino acid sequence of SEQ ID NO: 40, and/or a VL comprising an amino acid sequence of SEQ ID NO: 41;
(viii) an anti-KIR binding domain, wherein optionally the anti-KIR binding domain comprises a VH comprising an amino acid sequence of SEQ ID NO: 42, and/or a VL comprising an amino acid sequence of SEQ ID NO: 43;
(viii) an anti-CD56 binding domain, wherein optionally the anti-CD56 binding domain comprises a VH comprising an amino acid sequence of SEQ ID NO: 44, and/or a VL comprising an amino acid sequence of SEQ ID NO: 45;
(ix) an anti-CD226 binding domain, wherein optionally (i) the anti-CD226 binding domain comprises a VH comprising an amino acid sequence of SEQ ID NO: 46, and/or a VL comprising an amino acid sequence of SEQ ID NO: 47; or (ii) the anti-CD226 binding domain comprises a VH comprising an amino acid sequence of SEQ ID NO: 48, and/or a VL comprising an amino acid sequence of SEQ ID NO: 49;
(x) the antibody is an anti-CD25 binding domain, wherein optionally (i) the anti-CD25 binding domain comprises a VH comprising an amino acid sequence of SEQ ID NO: 50, and/or a VL comprising an amino acid sequence of SEQ ID NO: 51; or (ii) the anti-CD226 binding domain comprises a VH comprising an amino acid sequence of SEQ ID NO: 52, and/or a VL comprising an amino acid sequence of SEQ ID NO: 53;
(xi) an anti-CD83 binding domain, wherein optionally the anti-CD83 binding domain comprises a VH comprising an amino acid sequence of SEQ ID NO: 54, and/or a VL comprising an amino acid sequence of SEQ ID NO: 55;
(xii) an anti-KLRG1 binding domain, wherein optionally the anti-KLRG1 binding domain comprises a VH comprising an amino acid sequence of SEQ ID NO: 56, and/or a VL comprising an amino acid sequence of SEQ ID NO: 57;
(xiii) an anti-CD70 binding domain, wherein optionally the anti-CD70 binding domain comprises a VH comprising an amino acid sequence of SEQ ID NO: 58, and/or a VL comprising an amino acid sequence of SEQ ID NO: 59;
(xiv) an anti-CD30 binding domain, wherein optionally the anti-CD30 binding domain comprises a VH comprising an amino acid sequence of SEQ ID NO: 60, and/or a VL comprising an amino acid sequence of SEQ ID NO: 61;
(xv) an anti-CD229 binding domain, wherein optionally the anti-CD229 binding domain comprises a VH comprising an amino acid sequence of SEQ ID NO: 62, and/or a VL comprising an amino acid sequence of SEQ ID NO: 63; or
(xvi) an anti-NCR3 binding domain, wherein optionally the anti-NCR3 binding domain comprises a VH comprising an amino acid sequence of SEQ ID NO: 64, and/or a VL comprising an amino acid sequence of SEQ ID NO: 65.

In some embodiments, the first extracellular binding domain comprises a monospecific binding domain. In some embodiments, the first extracellular binding domain comprises a multispecific or multivalent binding domain.

In yet another aspect, provided herein is an engineered immune cell (such as an engineered T cell) comprising a functional exogenous receptor comprising an extracellular binding domain, a transmembrane domain, and an intracellular signaling domain, wherein the extracellular binding domain comprises (i) a first binding domain capable of binding a first antigen on natural killer (NK) cells, and (ii) a second binding domain capable of binding a second antigen, wherein the engineered immune cell (such as an engineered T cell) has reduced MHC I on cell surface; wherein the engineered immune cell (such as an engineered T cell)s is capable of depleting NK cells; and wherein optionally the endogenous expression of the first antigen is down regulated in the engineered immune cell (such as an engineered T cell). In some embodiments, the first antigen is also a cell surface marker on an activated T cell.

In some embodiments, the expression of the MHC I is reduced by expressing MHC I knockdown molecule on the engineered immune cell. In some embodiments, the MHC I knockdown molecule is sICP47 protein. In some embodiments, the sICP47 protein is derived from ICP47 of Simian Agent 8 (SA8), Cercopithecine herpesvirus 16 (CeHV-16), Cercopithecine herpesvirus 1 (CeHV-1), Macacine alphaherpesvirus 1, Pappine alphaherpesvirus 2 or functional variant thereof. In some embodiments, the sICP47 protein comprises an amino acid sequence selected from the group consisting of SEQ ID NOs: 10-15 or variants thereof.

In some embodiments, the functional exogenous receptor is a chimeric antigen receptor (CAR).

In some embodiments, the first antigen is selected from a group consisting of CD38, CS1, IL-T2, CD137, NKG2A, NKG2D. CD16. CD56. CD138. CD25. CD69, SLAM family members, L-SELECTIN, CD226, Kir family members, TIGIT. TRAIL, and the natural cytotoxicity receptors NCR1, NCR2, NCR3, wherein optionally SLAM family members are selected from a group consisting of SLAMF1, SLAMF4(2B4), SLAMF6, SLAMF3 and SLAMF5, and wherein optionally Kir family members are selected from a group consisting of KIR2DL1, KIR2DS1, KIR2DL2/L3, KIR2DS2, KIR2DS4, KIR3DL1, and KIR3DL2.

In some embodiments, the first binding domain comprises:
(i) an anti-CD38 binding domain, wherein optionally (i) the anti-CD38 binding domain comprises a VH comprising an amino acid sequence of SEQ ID NO: 1, and/or a VL comprising an amino acid sequence of SEQ ID NO: 2; or (ii) the anti-CD38 binding domain comprises a VH comprising an amino acid sequence of SEQ ID NO: 3, and/or a VL comprising an amino acid sequence of SEQ ID NO: 4;
(ii) an anti-CS1 binding domain, wherein optionally the anti-CS1 binding domain comprises a VH comprising an amino acid sequence of SEQ ID NO: 5, and/or a VL comprising an amino acid sequence of SEQ ID NO: 6;
(iii) an anti-IL-T2 binding domain, wherein optionally the anti-IL-T2 binding domain comprises a VH comprising an amino acid sequence of SEQ ID NO: 28, and/or a VL comprising an amino acid sequence of SEQ ID NO: 29;
(iv) an anti-CD137 binding domain, wherein optionally the anti-CD137 binding domain comprises a VH comprising an amino acid sequence of SEQ ID NO: 30, and/or a VL comprising an amino acid sequence of SEQ ID NO: 31;
(v) an anti-NKG2A binding domain, wherein optionally the anti-NKG2A antibody comprises a VH comprising an amino acid sequence of SEQ ID NO: 32, and/or a VL comprising an amino acid sequence of SEQ ID NO: 33;
(vi) an anti-NKG2D binding domain, wherein optionally (i) the anti-NKG2D binding domain comprises a VH comprising an amino acid sequence of SEQ ID NO: 34, and/or a VL comprising an amino acid sequence of SEQ ID NO: 35; or (ii) the anti-NKG2D binding domain comprises a VH comprising an amino acid sequence of SEQ ID NO: 36, and/or a VL comprising an amino acid sequence of SEQ ID NO: 37;
(vii) an anti-CD16 binding domain, wherein optionally the anti-CD16 binding domain comprises a VH comprising an amino acid sequence of SEQ ID NO: 38, and/or a VL comprising an amino acid sequence of SEQ ID NO: 39;
(viii) an anti-CD16a binding domain, wherein optionally the anti-CD16a binding domain comprises a VH comprising an amino acid sequence of SEQ ID NO: 40, and/or a VL comprising an amino acid sequence of SEQ ID NO: 41.
(ix) an anti-KIR binding domain, wherein optionally the anti-KIR binding domain comprises a VH comprising an amino acid sequence of SEQ ID NO: 42, and/or a VL comprising an amino acid sequence of SEQ ID NO: 43;
(x) an anti-CD56 binding domain, wherein optionally the anti-CD56 binding domain comprises a VH comprising an amino acid sequence of SEQ ID NO: 44, and/or a VL comprising an amino acid sequence of SEQ ID NO: 45;
(xi) an anti-CD226 binding domain, wherein optionally (i) the anti-CD226 binding domain comprises a VH comprising an amino acid sequence of SEQ ID NO: 46, and/or a VL comprising an amino acid sequence of SEQ ID NO: 47; or (ii) the anti-CD226 binding domain comprises a VH comprising an amino acid sequence of SEQ ID NO: 48, and/or a VL comprising an amino acid sequence of SEQ ID NO: 49;
(xii) the antibody is an anti-CD25 binding domain, wherein optionally (i) the anti-CD25 binding domain comprises a VH comprising an amino acid sequence of SEQ ID NO: 50, and/or a VL comprising an amino acid sequence of SEQ ID NO: 51; or (ii) the anti-CD226 binding domain comprises a VH comprising an amino acid sequence of SEQ ID NO: 52, and/or a VL comprising an amino acid sequence of SEQ ID NO: 53;
(xiii) an anti-CD83 binding domain, wherein optionally the anti-CD83 binding domain comprises a VH comprising an amino acid sequence of SEQ ID NO: 54, and/or a VL comprising an amino acid sequence of SEQ ID NO: 55;
(xiv) an anti-KLRG1 binding domain, wherein optionally the anti-KLRG1 binding domain comprises a VH comprising an amino acid sequence of SEQ ID NO: 56, and/or a VL comprising an amino acid sequence of SEQ ID NO: 57;
(xv) an anti-CD70 binding domain, wherein optionally the anti-CD70 binding domain comprises a VH comprising an amino acid sequence of SEQ ID NO: 58, and/or a VL comprising an amino acid sequence of SEQ ID NO: 59;
(xvi) an anti-CD30 binding domain, wherein optionally the anti-CD30 binding domain comprises a VH comprising an amino acid sequence of SEQ ID NO: 60, and/or a VL comprising an amino acid sequence of SEQ ID NO: 61;
(xvii) an anti-CD229 binding domain, wherein optionally the anti-CD229 binding domain comprises a VH comprising an amino acid sequence of SEQ ID NO: 62, and/or a VL comprising an amino acid sequence of SEQ ID NO: 63; or
(xviii) an anti-NCR3 binding domain, wherein optionally the anti-NCR3 binding domain comprises a VH comprising an amino acid sequence of SEQ ID NO: 64, and/or a VL comprising an amino acid sequence of SEQ ID NO: 65.

In some embodiments, the first binding domain comprises a monospecific binding domain. In some embodiments, the first binding domain comprises a multispecific or multivalent binding domain.

In some embodiments, the second antigen is a tumor antigen. In some embodiments, the tumor antigen is BCMA. In some embodiments, the antigen binding domain comprises an amino acid sequence of SEQ ID NO: 7, SEQ ID NO: 8 and/or SEQ ID NO: 9.

In some embodiments, the engineered immune cell is a T cell, a natural killer cell, a macrophage, a peripheral blood mononuclear cell (PBMC), a monocyte, a neutrophil, or an eosinophil. In some embodiments, the T cell is a cytotoxic T cell, a helper T cell, a natural killer T cell, or a γδT cell.

In yet another aspect, provided herein is a pharmaceutical composition, comprising the engineered immune cell (such as an engineered T cell) provided herein, and a pharmaceutically acceptable excipient.

In yet another aspect, provided herein is a method for treating a disease or disorder in a subject comprising administering to the subject a therapeutically effective amount of the engineered immune cell (such as an engineered T cell), or the pharmaceutical composition provided herein. In some embodiments, the disease or disorder is cancer. In some embodiments, the cancer is blood cancer. In other embodiments, the cancer is solid tumor cancer. In some embodiments, the subject is a human subject in need thereof.

### 4. BRIEF DESCRIPTION OF THE FIGURES

FIG. 1 shows the expression of CD38 in MM tumor cell line NCI-H929 cells and activated NK cells. MM tumor cell line NCI-H929 cells and activated NK cells were stained with anti-CD38 antibody, the NCI-H929 cells showed 97.4% CD38⁺ population, and active NK showed 85.3% CD38⁺ population.

FIG. 2 shows that in vitro treatment of PBMC with Daratumumab decreased the percentage of NK cell population.

FIG. 3 shows that anti-CD38 antibodies depleted NK cells in mice.

FIG. 4 shows in vivo treatment with Daratumumab in NCG mice helps the survival of MHC I KD/KO T cells.

FIGS. 5A-5C show the results of the MLR assays PBMCs (HLA-A2⁺ donor) and allogeneic T cells (HLA-A2⁻ donor). FIG. 5A shows the percentages of HLA-A2⁺ cells for unT, B2M KO T and M08+CD38&BCMA CAR-T (CD38 KO). FIG. 5B shows the FACS analysis data of day 2 and day 4. FIG. 5C shows the FACS analysis data of day 6. FIG. 5D shows the results of graft T cells in vivo HVG model (conducted with PBMCs (HLA-A2⁺ donor) as host cells and allogeneic T cells (HLA-A2− donor, including unT (CD38 KO), BCMA CAR-T, CD38/BCMA CAR-T (CD38 KO), and M08CD38/BCMA CAR-T (CD38 KO) as graft cells.

FIGS. 6A-6B show the results of the MLR assays conducted with PBMCs (HLA-A2-donor) and allogeneic T cells (HLA-A2⁺ donor). FIG. 6A shows depletion of B2M KO T cells was observed at day 6, and CD38 CAR-T cells were also rejected by allo-PBMC cells (from day 2 to day 6: 14.2% to 7.67%); whereas M08+CD38&BCMA CAR-T cells expanded to 70.5% at day 8. FIG. 6B shows that M08+CD38&BCMA CAR-T cells enriched during the MLR assay.

FIG. 7 shows the in vitro anti-tumor efficacy of CD38&BCMA CAR-T cells.

FIG. 8 shows the results of the MLR assays conducted with PBMCs (HLA-A2⁻ donor) and allogeneic NK cells (HLA-A2⁺ donor). By the treatment of CD38 antibody (Daratumumab), the host rejection from PBMCs were well controlled.

FIG. 9 shows the surface expression of CD56, CD16, 2B4, NCR1, NCR2, NCR3, KIR2DL1, KIR2DL2/L3, CD226, NKG2A, NKG2D, TIGIT, CD25, CD69 L-selectin and TRAIL on activated NK cells by FACS analysis.

FIG. 10A shows anti-alloreaction effects of M08+IL-T2/BCMA CAR-T cells and unT cells when co-cultured with allogeneic NK cells. FIG. 10B shows the results of the HVG MLR assays conducted with PBMCs (HLA-A2⁻ donor) and allogeneic unT cells and M08+IL-T2/BCMA CAR-T cells (HLA-A2⁺ donor).

FIG. 11A shows anti-alloreaction effects of CD137 CAR-T, M08+CD137 CAR-T, NKG2A CAR-T, M08+NKG2A CAR-T cells, M08+NKG2D-1 CAR-T, M08+NKG2D-2 CAR-T, M08+NCR3 CAR-T, M08+CD16 CAR-T, M08+CD16a CAR-T, M08+Kir CAR-T, M08+CD56 CAR-T and unT cells when co-cultured with allogeneic NK cells. FIG. 11B shows anti-alloreaction effects of M08+CD226-1/BCMA CAR-T, M08+CD226-2/BCMA CAR-T, M08+CD25-1/BCMA CAR-T cells, M08+CD25-2/BCMA CAR-T, M08+CD83/BCMA CAR-T, M08+KLRG1/BCMA CAR-T and unT cells when co-cultured with allogeneic NK cells.

FIG. 12 shows the results of the MLR assays conducted with PBMCs (HLA-A2⁺ donor) and allogeneic T cells (HLA-A2⁻ donor) and M08+NKG2A CAR-T cells (HLA-A2-donor). On day 4 after co-culture, CD56+/CD3− cells and CD56+/CD3+ cells in PBMCs were depleted by M08+NKG2A CAR-T cells.

FIG. 13A shows the CAR percentage of M08+CD70 CAR-T, M08+IL-T2 CAR-T, M08+CD30 CAR-T and M08+CD229 CAR T cells at day 7 and Day 14. CAR % cells were enriched during cell culture. FIG. 13B shows the anti-alloreaction effects of M08+CD226-1/BCMA CAR-T, M08+CD226-2/BCMA CAR-T, M08+CD70/BCMA CAR-T and unT cells when co-cultured with allogeneic T cells.

### 5. DETAILED DESCRIPTION

The present disclosure is based, in part, on the surprising finding of improved expansion, survival, and/or function of engineered immune cells (e.g., CAR-T cells) when depletion of recipient's immune cells (such as NK or T cells) involoved in immune rejection is used in conjunction with reduced expression of MHC I on the engineered immune cells. Such a combination can be achieved by using a prior treatment (i.e. depletion) with a therapeutic agent targeting immune cells (e.g. NK cells) before the administration of engineered immune cells; and/or by introducing into the engineered immune cells an exogenous moiety targeting immune cells (e.g. NK cells), for example, using an engineered immune cell (e.g., CAR-T cells) which has a functional exogenous receptors comprising two extracellular binding domains (e.g. a tandem CAR) or has two functional exogenous receptors (e.g. a dual CAR).

An important challenge for allogeneic cellular therapies is rejection of the infused engineered immune cells by the recipient's immune system because they are not HLA-matched. The immune response that results in graft rejection is a complex phenomenon, with respect both to the manner in which the graft antigens are presented to, and recognized by. See Graham et al., Cells, 7 (10) (2018); Yinmeng et al., Curr Opin Hematol., 22(6): 509-515 (2015); Pervinder et al., Front Immunol., 13(3):184 (2012); and Margaret, Graft rejection. Encyclopedia of Immunology, ISBN: 0-12-226765-6 (1998).

Allogeneic MHC molecule on a foreign cell can be recognized by a single T cell receptor (TCR), which is focused on exposed amino acid polymorphisms of the allogeneic MHC molecule independent of the peptide bound to it. Cytotoxic CD8 T-cell responses directed against MHC class I alloantigens are the principal arm of the cellular response against a transplanted organ. See Rocha et al., Immunol Rev., 196:51-64 (2003); and Le Moine et al., Transplantation, 73(9):1373-1381 (2002). Recipient dendritic cells present acquired MHC alloantigen both as intact protein, for recognition by cytotoxic CD8 T cells, and as processed allopeptide, for recognition by helper CD4 T cells. See Simon et al., PNAS, 112(41): 12788-12793 (2005).

Mechanisms involved in host immunity recognition of alloantigens is very complicate. Allo-MHC molecules can create many new pMHC complexes that can serve as ligands for various T cell clones. See Curr Opin Hematol., 22(6): 509-515 (2015); Pervinder et al., Front Immunol., 13(3):184 (2012). The prevalence of either model in T cell allorecognition presumably depends upon the degree of heterogeneity (structural and/or conformational) between recipient and donor MHC molecules. Induction of the adaptive immune response to an allograft begins with recognition of alloantigen by recipient T cells and occurs through three main processes known as the direct, the indirect, and the semi-direct pathways of antigen presentation. By MHC I knockout (KO) or knock-down (KD), the rejection from T cells may be avoid or reduced. However, the rejection by the recipient's immune system cannot be eliminated by MHC I knockout or knock-down. See Nowak et al., PLoS ONE, 7(9):e44718 (2012); Totterman et al., Transplantation, 47:817-823 (1989); Karre et al., Nature, 319:675-678 (1986); and Vampa et al., Transplantation, 7:1220-1228 (2003). For example, currently existing allogeneic CAR-T cells cannot last inside the body as long as their autologous counterparts. See, e.g., Karen et al., Clin Cancer Res, 24(24) (2018). Because the survival of a certain amount of CAR-T cells in the body plays a decisive role in its efficacy in vivo, there is clearly a need for improved allogeneic therapies.

In this context, the present disclosure makes a surprising finding that engineered T cells have much improved expansion and survival when an exogenous NK cell targeting moiety is introduced into the engineered T cells or when a treatment to a recipient with a NK cell targeting moiety is used prior to the infusion of the engineered T cells. Without being bound with any particular theories, another important immune cells involved in immune rejection are NK cells; so allo-reactive NK cells may also have a key role in immune response mechanisms elicited by the allograft. NK cells are innate lymphoid cells that control viral infections and tumors through cytotoxicity and production of cytokines such as IFN-γ. Loss of MHC-I may relieve inhibitory signals, allowing NK cell activation. Therefore, NK cells may complement T cell immunity by killing infected and transformed cells that down-regulate MHC-I to evade MHC-I restricted T cells.

In one aspect, provided herein is a method for treating a disease or disorder in a subject comprising administering to the subject an engineered immune cell, wherein the method comprises depleting the subject's immune cells, and wherein the engineered immune cell has a reduced expression of the MHC I molecule on its surface. In some embodiments, the method comprises depleting the subject's NK cells. In some embodiments, the method comprises depleting the subject's T cells. In other embodiments, the method comprises depleting both NK cells and T cells in the subject. In another aspect, provided herein is an engineered immune cell comprising a means for depleting immune cells such as NK cells and/or T cells and the immune cells has a reduced expression of the MHC I molecule on its surface.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “functional exogenous receptor” as used herein, refers to an exogenous receptor (e.g., TCR, cTCR, TAC-like chimeric receptor, or CAR) that retains its biological activity after being introduced into a T cell. The biological activity include but are not limited to the ability of the exogenous receptor in specifically binding to a molecule, properly transducing downstream signals, such as inducing cellular proliferation, cytokine production and/or performance of regulatory or cytolytic effector functions.

The term “chimeric antigen receptor” or “CAR” as used herein refers to an artificially constructed hybrid protein or polypeptide containing a binding moiety (e.g. an antibody) linked to immune cell (e.g. T cell) signaling or activation domains. CARs can be synthetic receptors that retarget T cells to tumor surface antigens (Sadelain et al., Nat. Rev. Cancer 3(1):35-45 (2003); Sadelain et al., Cancer Discovery 3(4):388-398 (2013)). CARs can provide both antigen binding and immune cell activation functions onto an immune cell such as a T cell. CARs have the ability to redirect T-cell specificity and reactivity toward a selected target in a non-MHC-restricted manner, exploiting the antigen-binding properties of monoclonal antibodies. The non-MHC-restricted antigen recognition can give T-cells expressing CARs the ability to recognize an antigen independent of antigen processing, thus bypassing a mechanism of tumor escape.

Several “generations” of CARs have been developed. First-generation CAR T-cells utilize an intracellular domain from the CD3ζ-chain of the TCR, which provides so called ‘signal 1’, and induces cytotoxicity against targeted cells. Engagement and signaling via the CD3ζ chain is required for T-cell stimulation and proliferation but is not often sufficient for sustained proliferation and activity in the absence of a second signal or ‘signal 2’. Second-generation CARs were developed to enhance efficacy and persistence in vivo after reinfusion into a subject and contain an second costimulatory signaling domain (CD28 or 4-1BB) intracellular domain that functions to provide ‘signal 2’ to mitigate anergy and activation-induced cell death seen with first generation CAR T-cells. Third-generation CARs are further optimized by use of two distinct costimulatory domains in tandem, e.g., CD28/4-1BB/CD3ζ or CD28/OX-40/CD3ζ. (see, e.g., Yeku et al., 2016, Armored CAR T-cells: utilizing cytokines and pro-inflammatory ligands to enhance CAR T-cell anti-tumour efficacy. Biochem Soc Trans. 44(2):412). CARs have been further optimized or “armored” to secrete active cytokines or express costimulatory ligands that further improve efficacy and persistence. The CAR used in the present invention can be first-generation, second-generation, third-generation, or “armored” CARs.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T cell” refers to a T cell that expresses a CAR.

“T cell receptor” or “TCR” as used herein refers to endogenous or recombinant/engineered T cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR comprises a TCRγ polypeptide chain and a TCRS polypeptide chain. In some embodiments, the TCR specifically binds a tumor antigen. In some embodiments, the TCR specifically binds a tumor antigen/MHC. “TCR-T” refers to a T cell that expresses a recombinant/engineered TCR.

“TCR complex” as used herein refers to a complex of TCR and CD3. “TCR subunits” used herein refers to a subunit of the TCR complex, which include, for example, TCRα, TCRβ, TCRγ, TCRδ, CD3ε, CD3γ, CD3δ, and CD3θ.

The term “deplete” or “depleting” as used herein in the context of a type of cells refers to reducing the amount of the type of cells. The term “deplete” or “depleting” includes both complete elimination and partial reduction such as decreasing by about 10%, 20%, 30%, 40%, 50%, 60%, 70%, 80%, 85%, 90%, 91%, 92%, 93%, 94%, 95%, 96%, 97%, 98%, or 99%.

The term “down regulation” or “down regulated” or alike as used herein in connection with a protein refers to that the expression or an activity of the protein is reduced. In some embodiments, the expression or an antivity of the protein is completed eliminated. The term also includes the situation where the expression or an activity of the protein is partially decreased, for example, by about 10%, 20%, 30%, 40%, 50%, 60%, 70%, 80%, or 90%.

The term “antibody,” “immunoglobulin,” or “lg” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG. IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113. Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The affinity at one binding site does not always reflect the true strength of the interaction between an antibody and an antigen. A binding molecule or antigen binding domain that binds to or specifically binds to an antigen can be identified, for example, by immunoassays, Octet®, Biacore®, or other techniques known to those of skill in the art. A binding molecule or antigen binding domain that binds to an antigen includes one that is capable of binding the antigen with sufficient affinity such that the binding molecule is useful, for example, as a therapeutic and/or diagnostic agent in targeting the antigen.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see. Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boemer et al., J. Immunol. 147(1):86-95 (1991), and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled. e.g., mice (see, e.g., Jakobovits, Curr. Opin. Biotechnol. 6(5):561-66 (1995); Brüggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies. e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150.000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for p and a isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while p and E contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by well-known numbering systems, such as Kabat, AbM, Chothia, Contact, IMGT, or combinations thereof. The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat Numbering) | | | | |
| CDR H1 | H31--H35 | H26--H35 | H26--H32 | H30--H35 | |
| | (Chothia Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5 and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia. European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

The term “donor subject” or “donor” refers to herein a subject whose cells are being obtained for further in vitro engineering. The donor subject can be a patient that is to be treated with a population of cells (i.e., an autologous donor), or can be an individual who donates a blood sample (e.g., lymphocyte sample) that, upon generation of the population of cells, will be used to treat a different individual or patient (i.e., an allogeneic donor). Those subjects who receive the cells that were prepared by the present methods can be referred to as “recipient” or “recipient subject.”

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A. B, and/or C” is intended to encompass each of the following embodiments: A, B. and C; A, B, or C; A or C; A or B; B or C; A and C; A and B; B and C; A (alone); B (alone); and C (alone).

5.2. Pre-Treatment for Depleting Natural Killer Cells

In one aspect, provided herein is a method for treating a disease or disorder using an allogeneic cellular therapy, in which prior to administering the engineered immune cells (e.g., CAR-T cells), the subject is administered with a treatment (e.g., with a therapeutic antibody or antibody-drug conjugate) to deplete NK cells in recipients.

In some embodiments, provided herein is a method for treating a disease or disorder in a subject comprising first administering to the subject an agent for depleting NK cells and then administering to the subject an engineered immune cell (such as an engineered T cell) comprising a functional exogenous receptor such as CAR. In some embodiments, an engineered immune cell (such as an engineered T cell) has reduced MHC I on cell surface, e.g., by knocking down or knocking out MHC I molecule. In some embodiments, the antigen targeted by the antibody in the pre-treatment is down regulated in the engineered immune cell (such as an engineered T cell).

Engineered immune cells provided herein include but not limited to T cells (such as a cytotoxic T cell, a helper T cell, a natural killer T cell, or a γδT cell), NK cells, macrophages, peripheral blood mononuclear cells (PBMC), monocytes, neutrophils, eosinophils, and so on. Many aspects in the present disclosure are described in the context of T cells for convenience, but they also apply to other types of immune cells.

5.2.1. Pre-Treatment with NK Cell Depleting Antibodies

In some embodiments, this pre-treatment is an antibody based treatment using antibodies targeting an antigen expressed on NK cells, and thereby depleting NK cells in the recipient subject. In some embodiments, the antibody targets an antigen universally expressed on NK cells. In some embodiments, the antibodies can deplete NK cells via antibody-dependent cellular cytotoxicity (ADCC). In some embodiments, the antibody targets an antigen universally expressed on NK cells. In some embodiments, the antibodies can deplete NK cells via complement-dependent cytotoxicity (CDC). In some embodiments, the antibody targets an antigen universally expressed on NK cells. In some embodiments, the antibodies can deplete NK cells via antibody-dependent cellular phagocytosis (ADCP). In other embodiments, the antibodies can deplete NK cells via induced apoptosis. In yet other embodiments, the antibody is conjugated to a drug as an antibody-drug conjugate (ADC) and the drug is capable of depleting NK cells. In some embodiments, the antigen targeted by the antibody is also a cell surface antigen on an activated T cell such as CD38.

The antibodies provided herein can be monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies). An antibody can be human, humanized, chimeric and/or affinity matured, as well as an antibody from other species, for example, mouse, rabbit, llama, etc. The antibodies provided herein also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Antibodies may be agonistic antibodies or antagonistic antibodies. Antibodies may be neither agonistic nor antagonistic.

In certain embodiments, the antibodies provided herein can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric sequences may include humanized sequences.

In certain embodiments, the antibodies can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin.

In certain embodiments, the antibodies provided herein can comprise portions of a “fully human antibody” or “human antibody.”

In certain embodiments, the antibodies provided herein can comprise portions of a “recombinant human antibody.”

In certain embodiments, the binding molecules or antigen binding domains can comprise a portion of a “monoclonal antibody.”

In some embodiments, the antibody provided herein binds to an antigen (e.g., human CD38 or human CS1 or human CD56 or human CD138) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M. e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore® TM-2000, or the Biacore®TM-3000 system.

In some embodiments, the antibody is an anti-CD38 antibody. In some embodiments, the anti-CD38 antibody is derived from Daratumumab. In other embodiments, the anti-CD38 antibody is derived from Isatuximab. In some embodiments, the anti-CD38 antibody comprises a VH comprising an amino acid sequence of SEQ ID NO: 1, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 1. In some embodiments, the anti-CD38 antibody comprises a VL comprising an amino acid sequence of SEQ ID NO: 2, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 2. In other embodiments, the anti-CD38 antibody comprises a VH comprising an amino acid sequence of SEQ ID NO: 3 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 3. In other embodiments, the anti-CD38 antibody comprises a VL comprising an amino acid sequence of SEQ ID NO: 4 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 4. In some specific embodiments, the anti-CD38 antibody comprises a VH comprising an amino acid sequence of SEQ ID NO: 1, and/or a VL comprising an amino acid sequence of SEQ ID NO: 2. In other specific embodiments, the anti-CD38 antibody comprises a VH comprising an amino acid sequence of SEQ ID NO: 3, and/or a VL comprising an amino acid sequence of SEQ ID NO: 4.

In other embodiments, the antibody is an anti-CS1 antibody. In some embodiments, the anti-CS1 antibody is derived from Elotuzumab. In other embodiments, the anti-CS1 antibody comprises a VH comprising an amino acid sequence of SEQ ID NO: 5 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 5. In other embodiments, the anti-CS1 antibody comprises a VL comprising an amino acid sequence of SEQ ID NO: 6 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 6. In some embodiments, the anti-CS1 antibody comprises a VH comprising an amino acid sequence of SEQ ID NO: 5, and/or a VL comprising an amino acid sequence of SEQ ID NO: 6.

In some embodiments, the antibody is an anti-IL-T2 antibody. In some embodiments, the anti-IL-T2 antibody comprises a VH comprising an amino acid sequence of SEQ ID NO: 28 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 28. In some embodiments, the anti-IL-T2 antibody comprises a VL comprising an amino acid sequence of SEQ ID NO: 29 or an amino acid sequence having at least 75%, 8(0%, 85%, 86%, 87%, 88%, 89%, 90/o, 91%, 92%, 93%, 94%, 95%, 96%, 97%, 98%, 99%, or 100% sequence identity to SEQ ID NO: 29. In some embodiments, the anti-IL-T2 antibody comprises a VH comprising an amino acid sequence of SEQ ID NO: 28, and/or a VL comprising an amino acid sequence of SEQ ID NO: 29.

In some embodiments, the antibody is an anti-CD137 antibody. In some embodiments, the antibody comprises a VH comprising an amino acid sequence of SEQ ID NO: 30 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 30. In some embodiments, the antibody comprises a VL comprising an amino acid sequence of SEQ ID NO: 31 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 31. In some embodiments, the anti-CD137 antibody comprises a VH comprising an amino acid sequence of SEQ ID NO: 30, and/or a VL comprising an amino acid sequence of SEQ ID NO: 31.

In some embodiments, the antibody is an anti-NKG2A antibody. In some embodiments, the antibody comprises a VH comprising an amino acid sequence of SEQ ID NO: 32 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 32. In some embodiments, the antibody comprises a VL comprising an amino acid sequence of SEQ ID NO: 33 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 33. In some embodiments, the anti-NKG2A antibody comprises a VH comprising an amino acid sequence of SEQ ID NO: 32, and/or a VL comprising an amino acid sequence of SEQ ID NO: 33.

In some embodiments, the antibody is an anti-NKG2D antibody. In some embodiments, the antibody comprises a VH comprising an amino acid sequence of SEQ ID NO: 34 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 34. In some embodiments, the antibody comprises a VL comprising an amino acid sequence of SEQ ID NO: 35 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 35. In some embodiments, the anti-NKG2D antibody comprises a VH comprising an amino acid sequence of SEQ ID NO: 34, and/or a VL comprising an amino acid sequence of SEQ ID NO: 35. In some embodiments, the antibody comprises a VH comprising an amino acid sequence of SEQ ID NO: 36 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 36. In some embodiments, the antibody comprises a VL comprising an amino acid sequence of SEQ ID NO: 37 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 37. In some embodiments, the anti-NKG2D antibody comprises a VH comprising an amino acid sequence of SEQ ID NO: 36, and/or a VL comprising an amino acid sequence of SEQ ID NO: 37.

In some embodiments, the antibody is an anti-CD16 antibody. In some embodiments, the antibody comprises a VH comprising an amino acid sequence of SEQ ID NO: 38 or an amino acid sequence having at least 75%, 80%, 85%, 86%, 87%, 88%, 89%, 90%, 91%, 92%, 93%, 94%, 95%, 9%%, 97%, 98%, 99%, or 100% sequence identity to SEQ ID NO: 38. In some embodiments, the antibody comprises a VL comprising an amino acid sequence of SEQ ID NO: 39 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 39. In some embodiments, the anti-CD16 antibody comprises a VH comprising an amino acid sequence of SEQ ID NO: 38, and/or a VL comprising an amino acid sequence of SEQ ID NO: 39.

In some embodiments, the antibody is an anti-CD16a antibody. In some embodiments, the antibody comprises a VH comprising an amino acid sequence of SEQ ID NO: 40 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 40. In some embodiments, the antibody comprises a VL comprising an amino acid sequence of SEQ ID NO: 41 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 41. In some embodiments, the anti-CD16a antibody comprises a VH comprising an amino acid sequence of SEQ ID NO: 40, and/or a VL comprising an amino acid sequence of SEQ ID NO: 41.

In some embodiments, the antibody is an anti-KIR antibody. In some embodiments, the antibody comprises a VH comprising an amino acid sequence of SEQ ID NO: 42 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 42. In some embodiments, the antibody comprises a VL comprising an amino acid sequence of SEQ ID NO: 43 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 43. In some embodiments, the anti-KIR antibody comprises a VH comprising an amino acid sequence of SEQ ID NO: 42, and/or a VL comprising an amino acid sequence of SEQ ID NO: 43.

In some embodiments, the antibody is an anti-CD56 antibody. In some embodiments, the antibody comprises a VH comprising an amino acid sequence of SEQ ID NO: 44 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 44. In some embodiments, the antibody comprises a VL comprising an amino acid sequence of SEQ ID NO: 45 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 45. In some embodiments, the anti-CD56 antibody comprises a VH comprising an amino acid sequence of SEQ ID NO: 44, and/or a VL comprising an amino acid sequence of SEQ ID NO: 45.

In some embodiments, the antibody is an anti-CD226 antibody. In some embodiments, the antibody comprises a VH comprising an amino acid sequence of SEQ ID NO: 46 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 46. In some embodiments, the antibody comprises a VL comprising an amino acid sequence of SEQ ID NO: 47 or an amino acid sequence having at least 75%, 80%, 85%, 86%, 87%, 88%, 89%, 90° %, 91%, 92%, 93%, 94%, 95%, 96%, 97%, 98%, 99%, or 100% sequence identity to SEQ ID NO: 47. In some embodiments, the anti-CD226 antibody comprises a VH comprising an amino acid sequence of SEQ ID NO: 46, and/or a VL comprising an amino acid sequence of SEQ ID NO: 47. In some embodiments, the antibody comprises a VH comprising an amino acid sequence of SEQ ID NO: 48 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 48. In some embodiments, the antibody comprises a VL comprising an amino acid sequence of SEQ ID NO: 49 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 49. In some embodiments, the anti-CD226 antibody comprises a VH comprising an amino acid sequence of SEQ ID NO: 48, and/or a VL comprising an amino acid sequence of SEQ ID NO: 49.

In some embodiments, the antibody is an anti-CD25 antibody. In some embodiments, the antibody comprises a VH comprising an amino acid sequence of SEQ ID NO: 50 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 50. In some embodiments, the antibody comprises a VL comprising an amino acid sequence of SEQ ID NO: 51 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 51. In some embodiments, the anti-CD25 antibody comprises a VH comprising an amino acid sequence of SEQ ID NO: 50, and/or a VL comprising an amino acid sequence of SEQ ID NO: 51. In some embodiments, the antibody comprises a VH comprising an amino acid sequence of SEQ ID NO: 52 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 52. In some embodiments, the antibody comprises a VL comprising an amino acid sequence of SEQ ID NO: 53 or an amino acid sequence having at least 75%, 80%, 85%, 86%, 87%, 88%, 89%, 90%/c, 91%, 92%, 93%, 94%, 95%, 96%, 97%, 98%, 99%, or 100% sequence identity to SEQ ID NO: 53. In some embodiments, the anti-CD226 antibody comprises a VH comprising an amino acid sequence of SEQ ID NO: 52, and/or a VL comprising an amino acid sequence of SEQ ID NO: 53.

In some embodiments, the antibody is an anti-CD83 antibody. In some embodiments, the antibody comprises a VH comprising an amino acid sequence of SEQ ID NO: 54 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 54. In some embodiments, the antibody comprises a VL comprising an amino acid sequence of SEQ ID NO: 55 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 55. In some embodiments, the anti-CD83 antibody comprises a VH comprising an amino acid sequence of SEQ ID NO: 54, and/or a VL comprising an amino acid sequence of SEQ ID NO: 55.

In some embodiments, the antibody is an anti-KLRG1 antibody. In some embodiments, the antibody comprises a VH comprising an amino acid sequence of SEQ ID NO: 56 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 56. In some embodiments, the antibody comprises a VL comprising an amino acid sequence of SEQ ID NO: 57 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 57. In some embodiments, the anti-KLRG1 antibody comprises a VH comprising an amino acid sequence of SEQ ID NO: 56, and/or a VL comprising an amino acid sequence of SEQ ID NO: 57.

In some embodiments, the antibody is an anti-CD70 antibody. In some embodiments, the antibody comprises a VH comprising an amino acid sequence of SEQ ID NO: 58 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 58. In some embodiments, the antibody comprises a VL comprising an amino acid sequence of SEQ ID NO: 59 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 59. In some embodiments, the anti-CD70 antibody comprises a VH comprising an amino acid sequence of SEQ ID NO: 58, and/or a VL comprising an amino acid sequence of SEQ ID NO: 59.

In some embodiments, the antibody is an anti-CD30 antibody. In some embodiments, the antibody comprises a VH comprising an amino acid sequence of SEQ ID NO: 60 or an amino acid sequence having at least 75%, 80%, 85%, 86%, 87%, 88%, 89%, 90%, 91%, 92%, 93%, 94%, 95%, 9%%, 97%, 98%, 99%, or 100% sequence identity to SEQ ID NO: 60. In some embodiments, the antibody comprises a VL comprising an amino acid sequence of SEQ ID NO: 61 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 61. In some embodiments, the anti-CD30 antibody comprises a VH comprising an amino acid sequence of SEQ ID NO: 60, and/or a VL comprising an amino acid sequence of SEQ ID NO: 61.

In some embodiments, the antibody is an anti-CD229 antibody. In some embodiments, the antibody comprises a VH comprising an amino acid sequence of SEQ ID NO: 62 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 62. In some embodiments, the antibody comprises a VL comprising an amino acid sequence of SEQ ID NO: 63 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 63. In some embodiments, the anti-CD229 antibody comprises a VH comprising an amino acid sequence of SEQ ID NO: 62, and/or a VL comprising an amino acid sequence of SEQ ID NO: 63.

In some embodiments, the antibody is an anti-NCR3 antibody. In some embodiments, the antibody comprises a VH comprising an amino acid sequence of SEQ ID NO: 64 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 64. In some embodiments, the antibody comprises a VL comprising an amino acid sequence of SEQ ID NO: 65 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 65. In some embodiments, the anti-NCR3 antibody comprises a VH comprising an amino acid sequence of SEQ ID NO: 64, and/or a VL comprising an amino acid sequence of SEQ ID NO: 65.

In yet other embodiments, the antibody is an anti-CD56 antibody. In other embodiments, the antibody is an anti-CD138 antibody.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g. of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the pre-treatment with the present antibodies deplete at least 10% of NK cells in a recipient subject. In some embodiments, the pre-treatment with the present antibodies deplete at least 15% of NK cells in a recipient subject. In some embodiments, the pre-treatment with the present antibodies deplete at least 20% of NK cells in a recipient subject. In some embodiments, the pre-treatment with the present antibodies deplete at least 25% of NK cells in a recipient subject. In some embodiments, the pre-treatment with the present antibodies deplete at least 30% of NK cells in a recipient subject. In some embodiments, the pre-treatment with the present antibodies deplete at least 35% of NK cells in a recipient subject. In some embodiments, the pre-treatment with the present antibodies deplete at least 40% of NK cells in a recipient subject. In some embodiments, the pre-treatment with the present antibodies deplete at least 45% of NK cells in a recipient subject. In some embodiments, the pre-treatment with the present antibodies deplete at least 50% of NK cells in a recipient subject. In some embodiments, the pre-treatment with the present antibodies deplete at least 55% of NK cells in a recipient subject. In some embodiments, the pre-treatment with the present antibodies deplete at least 60% of NK cells in a recipient subject. In some embodiments, the pre-treatment with the present antibodies deplete at least 65% of NK cells in a recipient subject. In some embodiments, the pre-treatment with the present antibodies deplete at least 70% of NK cells in a recipient subject. In some embodiments, the pre-treatment with the present antibodies deplete at least 75% of NK cells in a recipient subject. In some embodiments, the pre-treatment with the present antibodies deplete at least 80% of NK cells in a recipient subject. In some embodiments, the pre-treatment with the present antibodies deplete at least 85% of NK cells in a recipient subject. In some embodiments, the pre-treatment with the present antibodies deplete at least 90% of NK cells in a recipient subject.

In some embodiments, two or more antibodies are administered to the subject to deplete NK cells prior to the cellular therapy.

5.2.2. Treatment with Engineered Immune Cells

The engineered immune cells (e.g., engineered T cells) provided in the present methods comprise a functional exogenous receptor such as a T cell receptor (TCR), a chimeric antigen receptor (CAR), a chimeric TCR (cTCR), or a T cell antigen coupler (TAC)-like chimeric receptor. In some embodiments, the engineered T cells are allogeneic T cells, e.g., from a healthy donor.

In some embodiments, the engineered T cell has reduced MHC I on cell surface, e.g., by knocking down or knocking out a MHC I molecule or interfering the presentation of MHC I or a portion thereof on cell surface. Any methods known in the art for down regulating MHC I may be used in the present disclosure. Various methods for down regulating MHC I are described in more detail in Section 5.6 below.

In some more specific embodiments, the functional exogenous receptor is a CAR comprising a polypeptide comprising: (a) an extracellular antigen binding domain; (b) a transmembrane domain; and (c) an intracellular signaling domain, each of which and additional regions are described in more detail below.

### Extracellular Antigen Binding Domain

The extracellular antigen binding domain of the CARs described herein comprises one or more antigen binding domains. In some embodiments, the extracellular antigen binding domain of the CAR provided herein is mono-specific. In other embodiments, the extracellular antigen binding domain of the CAR provided herein is multispecific. In other embodiments, the extracellular antigen binding domain of the CAR provided herein is multivalent. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. In a specific embodiment, the extracellular antigen binding domain of the present CARs comprise a single-chain Fv (sFv or scFv). In another specific embodiment, the extracellular antigen binding domain of the present CARs comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., VHH or V_{NAR}), binding molecules naturally devoid of light chains, single domains (such as V_{H} or V_{L}) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In certain embodiments, the extracellular antigen binding domain comprises multiple binding domains. In some embodiments, the extracellular antigen binding domain comprises multispecific antibodies or fragments thereof, e.g., an extracellular antigen binding domain comprising multiple binding domains (e.g., multiple VHHs) in tandem. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies of the present disclosure can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in the table below. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, BCMA, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-1), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

Additional non-limiting exemplary targets of the CARs provided herein include GPC2, CD276, Delta-like protein ligand 3 (DLL3), NY-ESO-1, melanoma associated antigen 4; survivin protein, synovial sarcoma X breakpoint protein 2. CD3, epidermal growth factor receptor (EGFR), erbb2 tyrosine kinase receptor, HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor, Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF. VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present CARs is CD19. In other specific embodiments, at least one target antigen of the present CARs is CD20. In yet other specific embodiments, at least one target antigen of the present CARs is CD22. In yet other specific embodiments, at least one target antigen of the present CARs is BCMA. In yet other specific embodiments, at least one target antigen of the present CARs is VEGFR2. In yet other specific embodiments, at least one target antigen of the present CARs is FAP. In yet other specific embodiments, at least one target antigen of the present CARs is EpCam. In yet other specific embodiments, at least one target antigen of the present CARs is GPC3. In yet other specific embodiments, at least one target antigen of the present CARs is CD133. In yet other specific embodiments, at least one target antigen of the present CARs is IL13Ra. In yet other specific embodiments, at least one target antigen of the present CARs is EGFRIII. In yet other specific embodiments, at least one target antigen of the present CARs is EphA2. In yet other specific embodiments, at least one target antigen of the present CARs is Muc1. In yet other specific embodiments, at least one target antigen of the present CARs is CD70. In yet other specific embodiments, at least one target antigen of the present CARs is CD123. In yet other specific embodiments, at least one target antigen of the present CARs is ROR1. In yet other specific embodiments, at least one target antigen of the present CARs is PSMA. In yet other specific embodiments, at least one target antigen of the present CARs is CD5. In yet other specific embodiments, at least one target antigen of the present CARs is GD2. In yet other specific embodiments, at least one target antigen of the present CARs is GAP. In yet other specific embodiments, at least one target antigen of the present CARs is CD33. In yet other specific embodiments, at least one target antigen of the present CARs is CEA. In yet other specific embodiments, at least one target antigen of the present CARs is PSCA. In yet other specific embodiments, at least one target antigen of the present CARs is Her2. In yet other specific embodiments, at least one target antigen of the present CARs is Mesothelin.

### Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the CAR comprises a transmembrane domain chosen from the transmembrane domain of an alpha, beta or zeta chain of aT-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CDlld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NCR2, NCR3, NCR1, NKG2D, and/or NKG2C.

In some specific embodiments, the transmembrane domain is derived from CD8α. In other specific embodiments, the transmembrane domain is derived from CD28α.

### Intracellular Signaling Domain

The intracellular signaling domain in the CARs provided herein is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM.” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3z, FcR gamma (FCERIG), FcR beta (Fc Epsilon Rib), CD3 gamma, CD3 delta, CD3 epsilon, CD5, CD22, CD79a, CD79b, and CD66d.

### Co-Stimulatory Signaling Domain

In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3z) and one or more co-stimulatory signaling domains. In some embodiments, the one or more co-stimulatory signaling domains and the primary intracellular signaling domain (such as cytoplasmic signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3z). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B. BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta. OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

In some embodiments, the co-stimulatory signaling domains are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Signal Peptide

In certain embodiments, the CARs provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain.

### Hinge Region

In some embodiments, the CARs provided herein comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor.

In some specific embodiments, the hinge domain is derived from CD8a. In some embodiments, the hinge domain is a portion of the hinge domain of CD88, e.g., a fragment containing at least 15 (e.g., 20, 25, 30, 35, or 40) consecutive amino acids of the hinge domain of CD8a. In other embodiments, the hinge domain is derived from CD28a. In some embodiments, the hinge domain is a portion of the hinge domain of CD28a, e.g., a fragment containing at least 15 (e.g., 20, 25, 30, 35, or 40) consecutive amino acids of the hinge domain of CD28a.

In some specific embodiments, the extracellular binding domain of the CAR comprises an antigen binding domain capable of binding a tumor antigen. In some embodiments, the tumor antigen is BCMA. In some embodiments, the antigen binding domain comprises an amino acid sequence of SEQ ID NO: 7, SEQ ID NO: 8 and/or SEQ ID NO: 9.

In certain embodiments, the CAR provided herein comprises amino acid sequences with certain percent identity relative to any one of the CARs exemplified in Section 6 below. In some embodiments, provided herein is a CAR comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of the CARs exemplified in Section 6 below.

In some embodiments, amino acid sequence modification(s) or substitutions of the CARs described herein are contemplated. Alterations (e.g., substitutions) may be made in CDRs, e.g., to improve antibody affinity. In some embodiments, substitutions, insertions, or deletions may occur within one or more CDRs so long as such alterations do not substantially reduce the ability of the antibody to bind antigen.

5.3. Engineered Immune Cells Comprising a NK Cell Targeting Moiety

In another aspect, provided herein is an engineered immune cell (e.g., an engineered T cell) comprising a moiety targeting an antigen on NK cells for depleting NK cells, in addition to a functional exogenous receptor such as a CAR, a TCR, a chimeric TCR (cTCR), or a TAC-like chimeric receptor. In some embodiments, the antigen on NK cells provided herein is also a cell surface antigen on an activated T cell (such as CD38).

In some embodiments, the moiety targeting NK cells is itself a functional exogenous receptor such as a CAR. Thus, in some embodiments, the engineered immune cells comprising a NK cell targeting moiety provided herein comprises two or more CARs (such as dual CAR-T cells), at least one of which comprises an extracellular domain targeting an antigen on NK cells for depleting NK cells in a recipient subject.

In other embodiments, the moiety targeting NK cells is one of two or more antigen binding domains in the extracellular domain of a multispecific CAR. Thus, in some embodiments, the engineered immune cells comprising a NK cell targeting moiety comprises a multispecific CAR comprising an antigen binding domain targeting an antigen on NK cells and at least another antigen binding domain targeting a second antigen such as a tumor antigen.

Exemplary dual CAR-T cells and multispecific CAR-T cells are described in more detail below.

In some embodiments, the engineered immune cell provided herein is an allogeneic T cell, e.g., from a healthy donor. In some embodiments, the engineered T cell has reduced MHC I on cell surface, e.g., by knocking down or knocking out MHC I molecule or interfering the presentation of MHC I or a portion thereof on cell surface. Various methods for down regulating MHC I are described in more detail in Section 5.6 below.

In some embodiments, the endogenous expression of the antigen on NK cells (e.g., CD38 or CS1) targeted by the present CAR-T cells is down regulated in the present engineered T cell, for example, by knocking down or knocking out this antigen in the engineered T cell. Down regulation of the antigen can be conducted using any known methods in the art, including the various methods described in Section 5.6 below.

5.3.1. Dual CAR-T Cells

Exemplary dual CAR-T cells provided herein comprises a first CAR targeting NK cells and a second CAR targeting a tumor antigen.

CARs Targeting NK Cells

In some embodiments, the CAR targeting NK cells provided herein comprises an extracellular domain capable of binding an antigen expressed on NK cells such as CD38, CS1, IL-T2, 4-1BB, NKG2A, NKG2D, CD16, NCR1, CD56, CD138, SLAM family members, CD226, Kir family members, TIGIT, and the natural cytotoxicity receptors NCR3 and NCR2, wherein optionally SLAM family members are selected from a group consisting of SLAMF1, SLAMF4(2B4), SLAMF6, SLAMF3 and SLAMF5, and wherein optionally Kir family members are selected from a group consisting of KIR2DL1, KIR2DS1, KIR2DL2/L3, KIR2DS2, KIR2DS4, KIR3DL1, and KIR3DL2.

In some embodiments, the antigen is CD38. In some embodiments, the antigen is CS1. In some embodiments, the antigen is IL-T2. In some embodiments, the antigen is 4-1BB. In some embodiments, the antigen is NKG2A. In some embodiments, the antigen is NKG2D. In some embodiments, the antigen is CD16. In some embodiments, the antigen is NCR1. In some embodiments, the antigen is CD56. In some embodiments, the antigen is CD138. In some embodiments, the antigen is from SLAM family members (such as SLAMF1, SLAMF4(2B4), SLAMF6, SLAMF3 and SLAMF5). In some embodiments, the antigen is CD226. In some embodiments, the antigen is from Kir family members (such as KIR2DL1, KIR2DS1, KIR2DL2/L3, KIR2DS2, KIR2DS4, KIR3DL1, and KIR3DL2). In some embodiments, the antigen is TIGIT. In some embodiments, the antigen is NCR3. In some embodiments, the antigen is NCR2.

In some embodiments, the extracellular antigen binding domain comprises an antibody or a fragment thereof. In a specific embodiment, the extracellular antigen binding domain of the present CARs comprise a single-chain Fv (sFv or scFv). In another specific embodiment, the extracellular antigen binding domain of the present CARs comprises one or more single domain antibodies (sdAbs) such as VHH domains. In some embodiments, the extracellular antigen binding domain comprises humanized antibodies or fragment thereof.

In some embodiments, the extracellular antigen binding domain comprises an anti-CD38 domain. In a specific embodiment, the extracellular domain comprises an antigen binding domain capable of binding human CD38 with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the anti-CD38 domain is derived from Daratumumab. In other embodiments, the anti-CD38 domain is derived from Isatuximab. In some embodiments, the anti-CD38 domain comprises a VH comprising an amino acid sequence of SEQ ID NO: 1, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 1. In some embodiments, the anti-CD38 domain comprises a VL comprising an amino acid sequence of SEQ ID NO: 2, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 2. In other embodiments, the anti-CD38 domain comprises a VH comprising an amino acid sequence of SEQ ID NO: 3 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 3. In other embodiments, the anti-CD38 domain comprises a VL comprising an amino acid sequence of SEQ ID NO: 4 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 4. In some specific embodiments, the anti-CD38 domain comprises a VH comprising an amino acid sequence of SEQ ID NO: 1, and/or a VL comprising an amino acid sequence of SEQ ID NO: 2. In other specific embodiments, the anti-CD38 domain comprises a VH comprising an amino acid sequence of SEQ ID NO: 3, and/or a VL comprising an amino acid sequence of SEQ ID NO: 4.

In some embodiments, the extracellular domain comprises an anti-CS1 domain. In a specific embodiment, the extracellular domain comprises an antigen binding domain capable of binding human CS1 with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the anti-CS1 domain is derived from Elotuzumab. In some embodiments, the anti-CS1 domain comprises a VH comprising an amino acid sequence of SEQ ID NO: 5, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 5. In some embodiments, the anti-CS1 domain comprises a VL comprising an amino acid sequence of SEQ ID NO: 6, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 6. In some specific embodiments, the anti-CS1 domain comprises a VH comprising an amino acid sequence of SEQ ID NO: 5, and/or a VL comprising an amino acid sequence of SEQ ID NO: 6.

In some embodiments, the binding domain is an anti-IL-T2 binding domain. In some embodiments, the anti-IL-T2 binding domain comprises a VH comprising an amino acid sequence of SEQ ID NO: 28 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 28. In some embodiments, the anti-IL-T2 binding domain comprises a VL comprising an amino acid sequence of SEQ ID NO: 29 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 29. In some embodiments, the anti-IL-T2 binding domain comprises a VH comprising an amino acid sequence of SEQ ID NO: 28, and/or a VL comprising an amino acid sequence of SEQ ID NO: 29.

In some embodiments, the binding domain is an anti-CD137 binding domain. In some embodiments, the binding domain comprises a VH comprising an amino acid sequence of SEQ ID NO: 30 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 30. In some embodiments, the binding domain comprises a VL comprising an amino acid sequence of SEQ ID NO: 31 or an amino acid sequence having at least 75%, 801%, 85%, 86%, 87%, 88%, 89%, 90%, 91%, 92%, 93%, 94%, 95%, 96%, 97%, 98%, 99%, or 100% sequence identity to SEQ ID NO: 31. In some embodiments, the anti-CD137 binding domain comprises a VH comprising an amino acid sequence of SEQ ID NO: 30, and/or a VL comprising an amino acid sequence of SEQ ID NO: 31.

In some embodiments, the binding domain is an anti-NKG2A binding domain. In some embodiments, the binding domain comprises a VH comprising an amino acid sequence of SEQ ID NO: 32 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 32. In some embodiments, the binding domain comprises a VL comprising an amino acid sequence of SEQ ID NO: 33 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 33. In some embodiments, the anti-NKG2A binding domain comprises a VH comprising an amino acid sequence of SEQ ID NO: 32, and/or a VL comprising an amino acid sequence of SEQ ID NO: 33.

In some embodiments, the binding domain is an anti-NKG2D binding domain. In some embodiments, the binding domain comprises a VH comprising an amino acid sequence of SEQ ID NO: 34 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 34. In some embodiments, the binding domain comprises a VL comprising an amino acid sequence of SEQ ID NO: 35 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 35. In some embodiments, the anti-NKG2D binding domain comprises a VH comprising an amino acid sequence of SEQ ID NO: 34, and/or a VL comprising an amino acid sequence of SEQ ID NO: 35. In some embodiments, the binding domain comprises a VH comprising an amino acid sequence of SEQ ID NO: 36 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 36. In some embodiments, the binding domain comprises a VL comprising an amino acid sequence of SEQ ID NO: 37 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 37. In some embodiments, the anti-NKG2D binding domain comprises a VH comprising an amino acid sequence of SEQ ID NO: 36, and/or a VL comprising an amino acid sequence of SEQ ID NO: 37.

In some embodiments, the binding domain is an anti-CD16 binding domain. In some embodiments, the binding domain comprises a VH comprising an amino acid sequence of SEQ ID NO: 38 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 38. In some embodiments, the binding domain comprises a VL comprising an amino acid sequence of SEQ ID NO: 39 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 39. In some embodiments, the anti-CD16 binding domain comprises a VH comprising an amino acid sequence of SEQ ID NO: 38, and/or a VL comprising an amino acid sequence of SEQ ID NO: 39.

In some embodiments, the binding domain is an anti-CD16a binding domain. In some embodiments, the binding domain comprises a VH comprising an amino acid sequence of SEQ ID NO: 40 or an amino acid sequence having at least 75%, 8(0%, 85%, 86%, 87%, 88%, 89%, 90%, 91%, 92%, 93%, 94%, 95%, %%, 97%, 98%, 99%, or 100% sequence identity to SEQ ID NO: 40. In some embodiments, the binding domain comprises a VL comprising an amino acid sequence of SEQ ID NO: 41 or an amino acid sequence having at least 75%, 80%, 85%, 86%, 87%, 88%, 89%, 90%, 91%, 92%, 93%, 94%, 95%, 9%%, 97%, 98%, 99%, or 100% sequence identity to SEQ ID NO: 41. In some embodiments, the anti-CD16a binding domain comprises a VH comprising an amino acid sequence of SEQ ID NO: 40, and/or a VL comprising an amino acid sequence of SEQ ID NO: 41.

In some embodiments, the binding domain is an anti-KIR binding domain. In some embodiments, the binding domain comprises a VH comprising an amino acid sequence of SEQ ID NO: 42 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 42. In some embodiments, the binding domain comprises a VL comprising an amino acid sequence of SEQ ID NO: 43 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 43. In some embodiments, the anti-KIR binding domain comprises a VH comprising an amino acid sequence of SEQ ID NO: 42, and/or a VL comprising an amino acid sequence of SEQ ID NO: 43.

In some embodiments, the binding domain is an anti-CD56 binding domain. In some embodiments, the binding domain comprises a VH comprising an amino acid sequence of SEQ ID NO: 44 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 44. In some embodiments, the binding domain comprises a VL comprising an amino acid sequence of SEQ ID NO: 45 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 45. In some embodiments, the anti-CD56 binding domain comprises a VH comprising an amino acid sequence of SEQ ID NO: 44, and/or a VL comprising an amno acid sequence of SEQ ID NO: 45.

In some embodiments, the binding domain is an anti-CD226 binding domain. In some embodiments, the binding domain comprises a VH comprising an amino acid sequence of SEQ ID NO: 46 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 46. In some embodiments, the binding domain comprises a VL comprising an amino acid sequence of SEQ ID NO: 47 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 47. In some embodiments, the anti-CD226 binding domain comprises a VH comprising an amino acid sequence of SEQ ID NO: 46, and/or a VL comprising an amino acid sequence of SEQ ID NO: 47. In some embodiments, the binding domain comprises a VH comprising an amino acid sequence of SEQ ID NO: 48 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 48. In some embodiments, the binding domain comprises a VL comprising an amino acid sequence of SEQ ID NO: 49 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 49. In some embodiments, the anti-CD226 binding domain comprises a VH comprising an amino acid sequence of SEQ ID NO: 48, and/or a VL comprising an amino acid sequence of SEQ ID NO: 49.

In some embodiments, the binding domain is an anti-CD25 binding domain. In some embodiments, the binding domain comprises a VH comprising an amino acid sequence of SEQ ID NO: 50 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 50. In some embodiments, the binding domain comprises a VL comprising an amino acid sequence of SEQ ID NO: 51 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 51. In some embodiments, the anti-CD25 binding domain comprises a VH comprising an amino acid sequence of SEQ ID NO: 50, and/or a VL comprising an amino acid sequence of SEQ ID NO: 51. In some embodiments, the binding domain comprises a VH comprising an amino acid sequence of SEQ ID NO: 52 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 52. In some embodiments, the binding domain comprises a VL comprising an amino acid sequence of SEQ ID NO: 53 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 53. In some embodiments, the anti-CD226 binding domain comprises a VH comprising an amino acid sequence of SEQ ID NO: 52, and/or a VL comprising an amino acid sequence of SEQ ID NO: 53.

In some embodiments, the binding domain is an anti-CD83 binding domain. In some embodiments, the binding domain comprises a VH comprising an amino acid sequence of SEQ ID NO: 54 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 54. In some embodiments, the binding domain comprises a VL comprising an amino acid sequence of SEQ ID NO: 55 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 55. In some embodiments, the anti-CD83 binding domain comprises a VH comprising an amino acid sequence of SEQ ID NO: 54, and/or a VL comprising an amino acid sequence of SEQ ID NO: 55.

In some embodiments, the binding domain is an anti-KLRG1 binding domain. In some embodiments, the binding domain comprises a VH comprising an amino acid sequence of SEQ ID NO: 56 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 56. In some embodiments, the binding domain comprises a VL comprising an amino acid sequence of SEQ ID NO: 57 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 57. In some embodiments, the anti-KLRG1 binding domain comprises a VH comprising an amino acid sequence of SEQ ID NO: 56, and/or a VL comprising an amino acid sequence of SEQ ID NO: 57.

In some embodiments, the binding domain is an anti-CD70 binding domain. In some embodiments, the binding domain comprises a VH comprising an amino acid sequence of SEQ ID NO: 58 or an amino acid sequence having at least 75%, 800%, 85%, 86%, 87%, 88%, 89%, 90%, 91%, 92%, 93%, 94%, 95%, 96%, 97%, 98%, 99%, or 100% sequence identity to SEQ ID NO: 58. In some embodiments, the binding domain comprises a VL comprising an amino acid sequence of SEQ ID NO: 59 or an amino acid sequence having at least 75%, 80%, 85%, 86%, 87%, 88%, 89%, 90%, 91%, 92%, 93%, 94%, 95%, %%, 97%, 98%, 99%, or 100% sequence identity to SEQ ID NO: 59. In some embodiments, the anti-CD70 binding domain comprises a VH comprising an amino acid sequence of SEQ ID NO: 58, and/or a VL comprising an amino acid sequence of SEQ ID NO: 59.

In some embodiments, the binding domain is an anti-CD30 binding domain. In some embodiments, the binding domain comprises a VH comprising an amino acid sequence of SEQ ID NO: 60 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 60. In some embodiments, the binding domain comprises a VL comprising an amino acid sequence of SEQ ID NO: 61 or an amino acid sequence having at least 75%, 80%, 85%, 86%, 87%, 88%, 89%, 90%, 91%, 92%, 93%, 94%, 95%, %%, 97%, 98%, 99%, or 100% sequence identity to SEQ ID NO: 61. In some embodiments, the anti-CD30 binding domain comprises a VH comprising an amino acid sequence of SEQ ID NO: 60, and/or a VL comprising an amino acid sequence of SEQ ID NO: 61.

In some embodiments, the binding domain is an anti-CD229 binding domain. In some embodiments, the binding domain comprises a VH comprising an amino acid sequence of SEQ ID NO: 62 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 62. In some embodiments, the binding domain comprises a VL comprising an amino acid sequence of SEQ ID NO: 63 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 63. In some embodiments, the anti-CD229 binding domain comprises a VH comprising an amino acid sequence of SEQ ID NO: 62, and/or a VL comprising an amino acid sequence of SEQ ID NO; 63.

In some embodiments, the binding domain is an anti-NCR3 binding domain. In some embodiments, the binding domain comprises a VH comprising an amino acid sequence of SEQ ID NO: 64 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 64. In some embodiments, the binding domain comprises a VL comprising an amino acid sequence of SEQ ID NO: 65 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO; 65. In some embodiments, the anti-NCR3 binding domain comprises a VH comprising an amino acid sequence of SEQ ID NO: 64, and/or a VL comprising an amino acid sequence of SEQ ID NO: 65.

### CARs Targeting Tumor Antigens

The extracellular antigen binding domain of the CAR targeting a second antigen (e.g., a tumor antigen) in the present dual CAR-T cells comprises one or more antigen binding domains. In some embodiments, the extracellular antigen binding domain comprises an antibody or a fragment thereof.

In some embodiments, the extracellular antigen binding domain comprises an antibody or a fragment thereof.

In a specific embodiment, the extracellular antigen binding domain of the present CARs comprise a single-chain Fv (sFv or scFv). In another specific embodiment, the extracellular antigen binding domain of the present CARs comprises one or more single domain antibodies (sdAbs) such as VHH domains. In some embodiments, the extracellular antigen binding domain comprises humanized antibodies or fragment thereof.

In certain embodiments, the extracellular antigen binding domain comprises multiple binding domains (e.g., tandem CAR). In some embodiments, the extracellular antigen binding domain comprises multispecific antibodies or fragments thereof. In other embodiments, the extracellular antigen binding domain comprises multivalent antibodies or fragments thereof. In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor, including, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

Additional non-limiting exemplary targets of the CARs provided herein include GPC2. CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor, HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA. Mucin 1, HLA antigen (such as HLA class I antigen A-2 alpha, HLA class I antigen A-II alpha, and HLA class II antigen), c-Met, hepatocyte growth factor receptor, K-Ras GTPase (KRAS). IL-15 receptor. Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present CARs is CD19. In other specific embodiments, at least one target antigen of the present CARs is CD20. In yet other specific embodiments, at least one target antigen of the present CARs is CD22. In yet other specific embodiments, at least one target antigen of the present CARs is BCMA. In yet other specific embodiments, at least one target antigen of the present CARs is VEGFR2. In yet other specific embodiments, at least one target antigen of the present CARs is FAP. In yet other specific embodiments, at least one target antigen of the present CARs is EpCam. In yet other specific embodiments, at least one target antigen of the present CARs is GPC3. In yet other specific embodiments, at least one target antigen of the present CARs is CD133. In yet other specific embodiments, at least one target antigen of the present CARs is IL13Ra. In yet other specific embodiments, at least one target antigen of the present CARs is EGFRIII. In yet other specific embodiments, at least one target antigen of the present CARs is EphA2. In yet other specific embodiments, at least one target antigen of the present CARs is Muc1. In yet other specific embodiments, at least one target antigen of the present CARs is CD70. In yet other specific embodiments, at least one target antigen of the present CARs is CD123. In yet other specific embodiments, at least one target antigen of the present CARs is ROR1. In yet other specific embodiments, at least one target antigen of the present CARs is PSMA. In yet other specific embodiments, at least one target antigen of the present CARs is CD5. In yet other specific embodiments, at least one target antigen of the present CARs is GD2. In yet other specific embodiments, at least one target antigen of the present CARs is GAP. In yet other specific embodiments, at least one target antigen of the present CARs is CD33. In yet other specific embodiments, at least one target antigen of the present CARs is CEA. In yet other specific embodiments, at least one target antigen of the present CARs is PSCA. In yet other specific embodiments, at least one target antigen of the present CARs is Her2. In yet other specific embodiments, at least one target antigen of the present CARs is Mesothelin. In some more specific embodiments, at least one antigen is selected from those listed above.

In certain embodiments, the CAR provided herein comprises amino acid sequences with certain percent identity relative to any one of the CARs exemplified in the Section 6 below. In some embodiments, provided herein is a CAR comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of that of the CARs exemplified in the Section 6 below.

In some embodiments, the tumor antigen is BCMA. In some embodiments, the antigen binding domain comprises an amino acid sequence of SEQ ID NO: 7, SEQ ID NO: 8 and/or SEQ ID NO: 9.

The CARs targeting NK cells and the CARs targeting a tumor antigen may also include a transmembrane domain, a hinge region, an intracellular signaling domain, a co-stimulatory domain, and/or a signal peptide, each of which is as described in Section 5.2.2 above.

Specifically, in certain embodiments, the CARs provided herein may comprise a signal peptide at the N-terminus of the polypeptide. In some embodiments, the signal peptide is derived from a molecule selected from the group consisting of CD8α. GM-CSF receptor α, and IgG1 heavy chain.

In some embodiments, the CARs provided herein comprise a hinge domain that is located between the extracellular antigen binding domain and the transmembrane domain. In some specific embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8a. In other embodiments, the hinge domain is derived from CD28α. In some embodiments, the hinge domain is a portion of the hinge domain of CD28α, e.g., a fragment containing at least 15 (e.g., 20, 25, 30, 35, or 40) consecutive amino acids of the hinge domain of CD28a.

The CARs of the present disclosure comprise a transmembrane domain that can be directly or indirectly fused to the extracellular antigen binding domain. In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR. HVEM (LIGHTR), SLAMF7, NKp80 (KLRFl), CD160, CD19, IL-2R beta, IL-2R gamma, IL-7R a, ITGAL, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Lv9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NCR2, NCR3, NCR1, NKG2D, and/or NKG2C. In some specific embodiments, the transmembrane domain is derived from CD8α. In some embodiments, the transmembrane domain is a transmembrane domain of CD28α.

The intracellular signaling domain in the CARs provided herein is responsible for activation of at least one of the normal effector functions of the immune effector cell expressing the CARs. In some embodiments, the primary intracellular signaling domain contains a signaling motif known as immunoreceptor tyrosine-based activation motif, or ITAM. Exemplary ITAM-containing primary cytoplasmic signaling sequences include those derived from CD3z, FcR gamma (FCER1G), FcR beta (Fc Epsilon Rib), CD3 gamma, CD3 delta, CD3 epsilon, CD5, CD22, CD79a, CD79b, and CD66d.

In some embodiments, the CAR comprises at least one co-stimulatory signaling domain. The co-stimulatory signaling domain of any co-stimulatory molecule may be compatible for use in the CARs described herein. Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1 BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1. HLA Class I, HLA-DR, Ikaros, Integrin alpha 4/CD49d. Integrin alpha 4 beta 1, Integrin alpha 4 beta 7/LPAM-1, LAG-3, TCL1A, TCL1B, CRTAM, DAP12. Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

### Polypeptides Comprising Dual CARs

In yet another aspect, provided herein is a polypeptide comprising a CAR targeting NK cells as described above and a CAR targeting a tumor antigen as described above, wherein the two CARs are linked by a peptide linker.

Any linkers that are cleavable in cells may be used in the present disclosure to link multiple CARs.

In some embodiments, the peptide linker is a 2A self-cleaving peptide. The members of 2A peptides are named after the virus in which they have been first described. For example, F2A, the first described 2A peptide, is derived from foot-and-mouth disease virus. The self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Self-cleaving peptides are found in members of the picomaviridae virus family, including aphthoviruses such as foot-and-mouth disease virus (FMDV), equine rhinitis A virus (ERAV), thosea asigna virus (TaV) and porcine teschovirus-1 (PTV-1) (see Donnelly et al., J. Gen. Virol., 82: 1027-101 (2001); Ryan et al., J. Gen. Virol., 72: 2727-2732 (2001)) and cardioviruses such as theilovirus (e.g., theiler's murine encephalomyelitis) and encephalomyocarditis viruses. The 2A peptides derived from FMDV, ERAV, PTV-1, and TaV are sometimes referred to as “F2A,” “E2A,” “P2A,” and “T2A,” respectively, and are included in the present disclosure, e.g., as described in Donnelly et al., J. Gen. Virol., 78: 13-21 (1997); Ryan and Drew, EMBO J., 13: 928-933 (1994); Szymczak et al., Nature Biotech., 5: 589-594 (2004); Hasegawa et al., Stem Cells, 25(7): 1707-12 (2007). In yet other embodiments, intein mediated protein splicing system is used herein, e.g., as described in Shah and Muir, Chem Sci., 5(1): 446-461 (2014) and Topilina and Mills, Mobile DNA, 5 (5) (2014). Other methods known in the art can also be used in the present constructs.

In some embodiments, the 2A self-cleaving peptide is selected from a group consisting of F2A, E2A, P2A, T2A, or variants thereof.

5.3.2. CAR-T Cells Comprising Multispecific CARs

Exemplary multispecific CAR-T cells provided herein comprises a multispecific CAR (e.g., a bispecific CAR) comprising an extracellular domain comprising an antigen binding domain targeting an antigen on NK cells and an antigen binding domain targeting a tumor antigen.

In some embodiments, the two or more antigen binding domains in the present multispecific CARs comprise an antibody or a fragment thereof.

### Antigen Binding Domains Targeting NK Cells

In some embodiments, the antigen expressed on NK cells is selected from a group consisting of CD38, CS1, 4-1BB, NKG2A, NKG2D, CD16, NCR1, CD56, CD138, SLAM family members (such as SLAMF1, SLAMF4(2B4), SLAMF6, SLAMF3 and SLAMF5), CD226, Kir family members (such as KIR2DL1, KIR2DS1, KIR2DL2/L3, KIR2DS2, KIR2DS4, KIR3DL1, and KIR3DL2), TIGIT, and the natural cytotoxicity receptors NCR3 and NCR2.

In some embodiment, the antigen is CD38. In some embodiment, the antigen is CS1. In some embodiments, the antigen is 4-1BB. In some embodiments, the antigen is NKG2A. In some embodiments, the antigen is NKG2D. In some embodiments, the antigen is CD16. In some embodiments, the antigen is NCR1. In some embodiments, the antigen is CD56. In some embodiments, the antigen is CD138. In some embodiments, the antigen is a SLAM family member (such as SLAMF1, SLAMF4(2B4), SLAMF6, SLAMF3 and SLAMF5). In some embodiments, the antigen is CD226. In some embodiments, the antigen is a Kir family member (such as KIR2DL1, KIR2DS1, KIR2DL2/L3, KIR2DS2, KIR2DS4, KIR3DL1, and KIR3DL2). In some embodiments, the antigen is TIGIT. In some embodiments, the antigen is NCR3. In some embodiments, the antigen is NCR2.

In some embodiments, the extracellular domain comprises an anti-CD38 domain. In a specific embodiment, the extracellular domain comprises an antigen binding domain capable of binding human CD38 with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M). In some embodiments, the anti-CD38 domain is derived from Daratumumab. In other embodiments, the anti-CD38 domain is derived from Isatuximab. In some embodiments, the anti-CD38 domain comprises a VH comprising an amino acid sequence of SEQ ID NO: 1, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 1. In some embodiments, the anti-CD38 domain comprises a VL comprising an amino acid sequence of SEQ ID NO: 2, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 2. In other embodiments, the anti-CD38 domain comprises a VH comprising an amino acid sequence of SEQ ID NO: 3 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 3. In other embodiments, the anti-CD38 domain comprises a VL comprising an amino acid sequence of SEQ ID NO: 4 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 4. In some specific embodiments, the anti-CD38 domain comprises a VH comprising an amino acid sequence of SEQ ID NO: 1, and/or a VL comprising an amino acid sequence of SEQ ID NO: 2. In other specific embodiments, the anti-CD38 domain comprises a VH comprising an amino acid sequence of SEQ ID NO: 3, and/or a VL comprising an amino acid sequence of SEQ ID NO: 4.

In some embodiments, the extracellular domain comprises an anti-CS1 domain. In a specific embodiment, the extracellular domain comprises an antigen binding domain capable of binding human CS1 with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the anti-CS1 domain is derived from Elotuzumab. In some embodiments, the anti-CS1 domain comprises a VH comprising an amino acid sequence of SEQ ID NO: 5, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 5. In some embodiments, the anti-CS1 domain comprises a VL comprising an amino acid sequence of SEQ ID NO: 6, or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 6. In some specific embodiments, the anti-CS1 domain comprises a VH comprising an amino acid sequence of SEQ ID NO: 5, and/or a VL comprising an amino acid sequence of SEQ ID NO: 6.

In some embodiments, the binding domain is an anti-IL-T2 binding domain. In some embodiments, the anti-IL-T2 binding domain comprises a VH comprising an amino acid sequence of SEQ ID NO: 28 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 28. In some embodiments, the anti-IL-T2 binding domain comprises a VL comprising an amino acid sequence of SEQ ID NO: 29 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 29. In some embodiments, the anti-IL-T2 binding domain comprises a VH comprising an amino acid sequence of SEQ ID NO: 28, and/or a VL comprising an amino acid sequence of SEQ ID NO: 29.

In some embodiments, the binding domain is an anti-CD137 binding domain. In some embodiments, the binding domain comprises a VH comprising an amino acid sequence of SEQ ID NO: 30 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 30. In some embodiments, the binding domain comprises a VL comprising an amino acid sequence of SEQ ID NO: 31 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 31. In some embodiments, the anti-CD137 binding domain comprises a VH comprising an amino acid sequence of SEQ ID NO: 30, and/or a VL comprising an amino acid sequence of SEQ ID NO: 31.

In some embodiments, the binding domain is an anti-NKG2A binding domain. In some embodiments, the binding domain comprises a VH comprising an amino acid sequence of SEQ ID NO: 32 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 32. In some embodiments, the binding domain comprises a VL comprising an amino acid sequence of SEQ ID NO: 33 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 33. In some embodiments, the anti-NKG2A binding domain comprises a VH comprising an amino acid sequence of SEQ ID NO: 32, and/or a VL comprising an amino acid sequence of SEQ ID NO: 33.

In some embodiments, the binding domain is an anti-NKG2D binding domain. In some embodiments, the binding domain comprises a VH comprising an amino acid sequence of SEQ ID NO: 34 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 34. In some embodiments, the binding domain comprises a VL comprising an amino acid sequence of SEQ ID NO: 35 or an amino acid sequence having at least 75%, 800%, 85%, 86%, 87%, 88%, 89%, 90%, 91%, 92%, 93%, 94%, 95%, 96%, 97%, 98%, 99%, or 100% sequence identity to SEQ ID NO: 35. In some embodiments, the anti-NKG2D binding domain comprises a VH comprising an amino acid sequence of SEQ ID NO: 34, and/or a VL comprising an amino acid sequence of SEQ ID NO: 35. In some embodiments, the binding domain comprises a VH comprising an amino acid sequence of SEQ ID NO: 36 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 36. In some embodiments, the binding domain comprises a VL comprising an amino acid sequence of SEQ ID NO: 37 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 37. In some embodiments, the anti-NKG2D binding domain comprises a VH comprising an amino acid sequence of SEQ ID NO: 36, and/or a VL comprising an amino acid sequence of SEQ ID NO: 37.

In some embodiments, the binding domain is an anti-CD16 binding domain. In some embodiments, the binding domain comprises a VH comprising an amino acid sequence of SEQ ID NO: 38 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 38. In some embodiments, the binding domain comprises a VL comprising an amino acid sequence of SEQ ID NO: 39 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 39. In some embodiments, the anti-CD16 binding domain comprises a VH comprising an amino acid sequence of SEQ ID NO: 38, and/or a VL comprising an amino acid sequence of SEQ ID NO: 39.

In some embodiments, the binding domain is an anti-CD16a binding domain. In some embodiments, the binding domain comprises a VH comprising an amino acid sequence of SEQ ID NO: 40 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 40. In some embodiments, the binding domain comprises a VL comprising an amino acid sequence of SEQ ID NO: 41 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 41. In some embodiments, the anti-CD16a binding domain comprises a VH comprising an amino acid sequence of SEQ ID NO: 40, and/or a VL comprising an amino acid sequence of SEQ ID NO: 41.

In some embodiments, the binding domain is an anti-KIR binding domain. In some embodiments, the binding domain comprises a VH comprising an amino acid sequence of SEQ ID NO: 42 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 42. In some embodiments, the binding domain comprises a VL comprising an amino acid sequence of SEQ ID NO: 43 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 43. In some embodiments, the anti-KIR binding domain comprises a VH comprising an amino acid sequence of SEQ ID NO: 42, and/or a VL comprising an amino acid sequence of SEQ ID NO: 43.

In some embodiments, the binding domain is an anti-CD56 binding domain. In some embodiments, the binding domain comprises a VH comprising an amino acid sequence of SEQ ID NO: 44 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 44. In some embodiments, the binding domain comprises a VL comprising an amino acid sequence of SEQ ID NO: 45 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 45. In some embodiments, the anti-CD56 binding domain comprises a VH comprising an amino acid sequence of SEQ ID NO: 44, and/or a VL comprising an amino acid sequence of SEQ ID NO: 45.

In some embodiments, the binding domain is an anti-CD226 binding domain. In some embodiments, the binding domain comprises a VH comprising an amino acid sequence of SEQ ID NO: 46 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 46. In some embodiments, the binding domain comprises a VL comprising an amino acid sequence of SEQ ID NO: 47 or an amino acid sequence having at least 75%, 8(0%, 85%, 86%, 87%, 88%, 89%, 90%, 91%, 92%, 93%, 94%, 95%, 96%, 97%, 98%, 99%, or 1000% sequence identity to SEQ ID NO: 47. In some embodiments, the anti-CD226 binding domain comprises a VH comprising an amino acid sequence of SEQ ID NO: 46, and/or a VL comprising an amino acid sequence of SEQ ID NO: 47. In some embodiments, the binding domain comprises a VH comprising an amino acid sequence of SEQ ID NO: 48 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 48. In some embodiments, the binding domain comprises a VL comprising an amino acid sequence of SEQ ID NO: 49 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 49. In some embodiments, the anti-CD226 binding domain comprises a VH comprising an amino acid sequence of SEQ ID NO: 48, and/or a VL comprising an amino acid sequence of SEQ ID NO: 49.

In some embodiments, the binding domain is an anti-CD25 binding domain. In some embodiments, the binding domain comprises a VH comprising an amino acid sequence of SEQ ID NO: 50 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 50. In some embodiments, the binding domain comprises a VL comprising an amino acid sequence of SEQ ID NO: 51 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 51. In some embodiments, the anti-CD25 binding domain comprises a VH comprising an amino acid sequence of SEQ ID NO: 50, and/or a VL comprising an amino acid sequence of SEQ ID NO: 51. In some embodiments, the binding domain comprises a VH comprising an amino acid sequence of SEQ ID NO: 52 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 52. In some embodiments, the binding domain comprises a VL comprising an amino acid sequence of SEQ ID NO: 53 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 53. In some embodiments, the anti-CD226 binding domain comprises a VH comprising an amino acid sequence of SEQ ID NO: 52, and/or a VL comprising an amino acid sequence of SEQ ID NO: 53.

In some embodiments, the binding domain is an anti-CD83 binding domain. In some embodiments, the binding domain comprises a VH comprising an amino acid sequence of SEQ ID NO: 54 or an amino acid sequence having at least 75%, 8(0%, 85%, 86%, 87%, 88%, 89%, 90%, 91%, 92%, 93%, 94%, 95%, 96%, 97%, 98%, 99%, or 100% sequence identity to SEQ ID NO: 54. In some embodiments, the binding domain comprises a VL comprising an amino acid sequence of SEQ ID NO: 55 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 55. In some embodiments, the anti-CD83 binding domain comprises a VH comprising an amino acid sequence of SEQ ID NO: 54, and/or a VL comprising an amino acid sequence of SEQ ID NO: 55.

In some embodiments, the binding domain is an anti-KLRG1 binding domain. In some embodiments, the binding domain comprises a VH comprising an amino acid sequence of SEQ ID NO: 56 or an amino acid sequence having at least 75%, 800%, 85%, 86%, 87%, 88%, 89%, 90%, 91%, 92%, 93%, 94%, 95%, 96%, 97%, 98%, 99%, or 100% sequence identity to SEQ ID NO: 56. In some embodiments, the binding domain comprises a VL comprising an amino acid sequence of SEQ ID NO: 57 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 57. In some embodiments, the anti-KLRG1 binding domain comprises a VH comprising an amino acid sequence of SEQ ID NO: 56, and/or a VL comprising an amino acid sequence of SEQ ID NO; 57.

In some embodiments, the binding domain is an anti-CD70 binding domain. In some embodiments, the binding domain comprises a VH comprising an amino acid sequence of SEQ ID NO: 58 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 58. In some embodiments, the binding domain comprises a VL comprising an amino acid sequence of SEQ ID NO: 59 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 59. In some embodiments, the anti-CD70 binding domain comprises a VH comprising an amino acid sequence of SEQ ID NO: 58, and/or a VL comprising an amino acid sequence of SEQ ID NO: 59.

In some embodiments, the binding domain is an anti-CD30 binding domain. In some embodiments, the binding domain comprises a VH comprising an amino acid sequence of SEQ ID NO: 60 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 60. In some embodiments, the binding domain comprises a VL comprising an amino acid sequence of SEQ ID NO: 61 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 61. In some embodiments, the anti-CD30 binding domain comprises a VH comprising an amino acid sequence of SEQ ID NO: 60, and/or a VL comprising an amino acid sequence of SEQ ID NO: 61.

In some embodiments, the binding domain is an anti-CD229 binding domain. In some embodiments, the binding domain comprises a VH comprising an amino acid sequence of SEQ ID NO: 62 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 62. In some embodiments, the binding domain comprises a VL comprising an amino acid sequence of SEQ ID NO: 63 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 63. In some embodiments, the anti-CD229 binding domain comprises a VH comprising an amino acid sequence of SEQ ID NO: 62, and/or a VL comprising an amino acid sequence of SEQ ID NO: 63.

In some embodiments, the binding domain is an anti-NCR3 binding domain. In some embodiments, the binding domain comprises a VH comprising an amino acid sequence of SEQ ID NO: 64 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 64. In some embodiments, the binding domain comprises a VL comprising an amino acid sequence of SEQ ID NO: 65 or an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 65. In some embodiments, the anti-NCR3 binding domain comprises a VH comprising an amino acid sequence of SEQ ID NO: 64, and/or a VL comprising an amino acid sequence of SEQ ID NO: 65.

### Antigen Binding Domains Targeting Tumor Antigens

Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, BCMA, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). Non-limiting examples of TSA or TAA antigens include: differentiation antigens such as MART-1/MelanA (MART-I), gp 100 (Pmel 17), tyrosinase. TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK. MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

Additional non-limiting exemplary targets of the CARs provided herein include GPC2, CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor. HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor. Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present CARs is CD19. In other specific embodiments, at least one target antigen of the present CARs is CD20. In yet other specific embodiments, at least one target antigen of the present CARs is CD22. In yet other specific embodiments, at least one target antigen of the present CARs is BCMA. In yet other specific embodiments, at least one target antigen of the present CARs is VEGFR2. In yet other specific embodiments, at least one target antigen of the present CARs is FAP. In yet other specific embodiments, at least one target antigen of the present CARs is EpCam. In yet other specific embodiments, at least one target antigen of the present CARs is GPC3. In yet other specific embodiments, at least one target antigen of the present CARs is CD133. In yet other specific embodiments, at least one target antigen of the present CARs is IL13Ra. In yet other specific embodiments, at least one target antigen of the present CARs is EGFRIII. In yet other specific embodiments, at least one target antigen of the present CARs is EphA2. In yet other specific embodiments, at least one target antigen of the present CARs is Muc1. In yet other specific embodiments, at least one target antigen of the present CARS is CD70. In yet other specific embodiments, at least one target antigen of the present CARs is CD123. In yet other specific embodiments, at least one target antigen of the present CARs is ROR1. In yet other specific embodiments, at least one target antigen of the present CARs is PSMA. In yet other specific embodiments, at least one target antigen of the present CARs is CD5. In yet other specific embodiments, at least one target antigen of the present CARs is GD2. In yet other specific embodiments, at least one target antigen of the present CARs is GAP. In yet other specific embodiments, at least one target antigen of the present CARs is CD33. In yet other specific embodiments, at least one target antigen of the present CARs is CEA. In yet other specific embodiments, at least one target antigen of the present CARs is PSCA. In yet other specific embodiments, at least one target antigen of the present CARs is Her2. In yet other specific embodiments, at least one target antigen of the present CARs is Mesothelin.

In some embodiments, the tumor antigen is BCMA. In some embodiments, the antigen binding domain comprises an amino acid sequence of SEQ ID NO: 7, SEQ ID NO: 8 and/or SEQ ID NO: 9.

The multispecific CARs provided herein also include a transmembrane domain, a hinge region, an intracellular signaling domain, a co-stimulatory domain, and/or a signal peptide, each of which is as described in Section 5.2.2 above. Briefly, in certain embodiments, the CARs provided herein may comprise a signal peptide at the N-terminus of the polypeptide. In some embodiments, the signal peptide is derived from a molecule selected from the group consisting of CD8α, GM-CSF receptor α, and IgG1 heavy chain. In some embodiments, the CARs provided herein comprise a hinge domain that is located between the extracellular antigen binding domain and the transmembrane domain. In some specific embodiments, the hinge domain is derived from CD8α. In other embodiments, the hinge domain is derived from CD28α. The CARs of the present disclosure comprise a transmembrane domain that can be directly or indirectly fused to the extracellular antigen binding domain. In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40. BAFFR, HVEM (LIGHTR), SLAMF7, NKp80 (KLRFI), CD60, CD19. IL-2R beta, IL-2R gamma, IL-7R a. ITGA1, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NCR2, NCR3, NCR1, NKG2D, and/or NKG2C. In some specific embodiments, the transmembrane domain is derived from CD8α. In some embodiments, the transmembrane domain is a transmembrane domain of CD28α. The intracellular signaling domain in the CARs provided herein is responsible for activation of at least one of the normal effector functions of the immune effector cell expressing the CARs. In some embodiments, the primary intracellular signaling domain contains a signaling motif known as immunoreceptor tyrosine-based activation motif, or ITAM. Exemplary ITAM-containing primary cytoplasmic signaling sequences include those derived from CD3z, FcR gamma (FCER1G), FcR beta (Fc Epsilon Rib), CD3 gamma. CD3 delta. CD3 epsilon, CD5, CD22, CD79a. CD79b, and CD66d. In some embodiments, the CAR comprises at least one co-stimulatory signaling domain. The co-stimulatory signaling domain of any co-stimulatory molecule may be compatible for use in the CARs described herein. Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand-TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta. OX40/TNFRSF4, OX40 Ligand/TNFSF4, RELT/TNFRSF9L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

### Peptide Linkers

The various binding domains in the multispecific CARs described herein may be fused to each other via peptide linkers. In some embodiments, the binding domains are directly fused to each other without any peptide linkers. The peptide linkers connecting different domains may be the same or different. Different domains of the CARs may also be fused to each other via peptide linkers.

Each peptide linker in a CAR may have the same or different length and/or sequence depending on the structural and/or functional features of the binding domains and/or the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. In some embodiments, a short peptide linker may be disposed between the transmembrane domain and the intracellular signaling domain of a CAR. In some embodiment, a peptide linker comprises flexible residues (such as glycine and serine) so that the adjacent domains are free to move relative to each other. For example, a glycine-serine doublet can be a suitable peptide linker.

In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers, glycine-serine polymers, glycine-alanine polymers, alanine-serine polymers, and other flexible linkers known in the art.

Other linkers known in the art, for example, as described in WO2016014789. WO2015158671, WO2016102965, US20150299317, WO02018067992, U.S. Pat. No. 7,741,465, Colcher el al., J Nat. Cancer Inst 82:1191-1197 (1990), and Bird et al., Science 242:423-426 (1988) may also be included in the CARs provided herein, the disclosure of each of which is incorporated herein by reference.

5.4. Polynucleotides

In another aspect, the disclosure provides polynucleotides that encode the polypeptide provided herein, including those antibodies, CARs, and fusion polypeptides described in Section 5.2 and Section 5.2 above. For example, in some embodiments, provided herein is a polynucleotide encoding a polypeptide comprising a first CAR targeting NK cells and a second CAR targeting a tumor antigen, wherein the first and the second CARs are linked via peptide linker such as a self-cleavable linker (e.g., 2A peptides).

In yet another aspect, provided herein is a polynucleotide comprising a first region encoding a first CAR targeting NK cells, and a second region encoding a second CAR targeting a tumor antigen. The NK cell targeting CARs and the tumor antigen targeting CARs are as described in Section 5.2 and Section 5.3 above. In some embodiments, the first region and the second region are controlled by the same promoter. For example, in some embodiments, internal ribosomal entry sites (IRES) are used herein to express multiple genes from one promoter. In other embodiments, the first region and the second region are controlled by separate promoters.

In yet another aspect, provided herein is a composition comprising a first polynucleotide encoding a first CAR targeting NK cells, and a second CAR targeting a tumor antigen. The NK cell targeting CARs and the tumor antigen targeting CARs are as described in Section 5.2 and Section 5.3 above.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the polypeptide of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about %%, 97%, 98% or 99% identical to a polynucleotide encoding the polypeptide of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

5.5. Vectors

Also provided are vectors comprising the polynucleotides or nucleic acid molecules described herein. In one embodiment, the nucleic acid molecules can be incorporated into a recombinant expression vector.

The present disclosure provides vectors for cloning and expressing any one of the polypeptides described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual. Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a polypeptide described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the polypeptide is operably linked to a constitutive promoter. Constitutive promoters allow heterologous genes (also referred to as transgenes) to be expressed constitutively in the host cells. Exemplary constitutive promoters contemplated herein include, but are not limited to, murine stem cell virus (MSCV) promoter, cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the CAR is operably linked to a hEF1α promoter. In some embodiments, the nucleic acid encoding the CAR is operably linked to a MSCV promoter.

In some embodiments, the nucleic acid encoding the polypeptide is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the polypeptide from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

5.6. Preparation of Engineered Immune Effector Cells

In another aspect, provided herein are methods or processes for making or producing the immune effector cells (e.g., engineered T cells) comprising any one of the polypeptides, polynucleotides, or vectors described herein.

In some embodiments, provided herein is a method of producing an engineered T cell comprising introducing into the T cell a polynucleotide or a vector provided herein as described in Section 5.4 and Section 5.5.

In some embodiments, provided herein is a method of producing an engineered T cell comprising introducing into the T cell a polynucleotide encoding a polypeptide comprising a first CAR targeting NK cells and a second CAR targeting a tumor antigen, wherein the first and the second CARs are linked via peptide linker such as a self-cleavable linker (e.g., 2A peptides).

In other embodiments, provided herein is a method of producing an engineered T cell comprising introducing into the T cell a polynucleotide comprising a first region encoding a first CAR targeting NK cells, and a second region encoding a second CAR targeting a tumor antigen. The NK cell targeting CARs and the tumor antigen targeting CARs are as described in Section 5.2 and Section 5.3 above. In some embodiments, the first region and the second region are controlled by the same promoter. For example, in some embodiments, internal ribosomal entry sites (IRES) are used herein to express multiple genes from one promoter. In other embodiments, the first region and the second region are controlled by separate promoters.

In other embodiments, provided herein is a method of producing an engineered T cell comprising introducing into the T cell a composition comprising a first polynucleotide encoding a first CAR targeting NK cells, and a second CAR targeting a tumor antigen. The NK cell targeting CARs and the tumor antigen targeting CARs are as described in Section 5.2 and Section 5.3 above.

In yet other embodiments, provided herein is a method of producing an engineered T cell comprising introducing into a T cell a polynucleotide encoding a multispecific CAR comprising a first antigen binding domain targeting NK cells and a second antigen binding domain targeting a tumor antigen as described in Section 5.3.2 above.

The engineered immune effector cells are prepared by introducing the polypeptide provided herein into the immune effector cells, such as T cells. In some embodiments, the polypeptide is introduced to the immune effector cells by transfecting any one of the isolated nucleic acids or any one of the vectors described above.

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the polypeptides described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the polypeptide in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In yet another aspect, provided herein is an engineered immune effector cell (e.g., a CAR-T cell) produced according to the method provided herein.

In some embodiments, the method or process provided herein comprises modifying the immune effector cells prior to introducing the exogenenous functional receptor (such as one or more CARs), for example, knocking down or knocking out one or more endogenous gene (e.g., the genes encoding MHC I molecule and/or an antigen on NK cells targeted by the CAR to be introduced into the immune cell). This and other steps are described in more detail below.

### Source of T Cells, Cell Preparation and Culture

Prior to expansion and genetic modification of the T cells (e.g., precursor T cells), a source of T cells is obtained from an individual. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. In some embodiments, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, the T cell is provided from an umbilical cord blood bank, a peripheral blood bank, or derived from an induced pluripotent stem cell (iPSC), multipotent and pluripotent stem cell, or a human embryonic stem cell. In some embodiments, the T cells are derived from cell lines. The T cells in some embodiments are obtained from a xenogeneic source, for example, from mouse, rat, non-human primate, and pig. In some embodiments, the T cells are human cells. In some aspects, the T cells are primary cells, such as those isolated directly from a subject and/or isolated from a subject and frozen.

In some embodiments, the cells include one or more subsets of T cells, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some cases, the T cell is allogeneic in reference to one or more intended recipients. In some cases, the T cell is suitable for transplantation, such as without inducing GvHD in the recipient.

Among the sub-types and subpopulations of T cells and/or of CD4⁺ and/or of CD8⁺ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62Lhi, GITR⁺, and FoxP3⁺. Alternatively, in certain embodiments. T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present application is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993).

In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Whether prior to or after genetic modification of the T cells with functional exogenous receptors described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells. In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8⁺ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15 (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresis peripheral blood mononuclear cell products have a helper T cell population (TH, CD4⁺) that is greater than the cytotoxic or suppressor T cell population (TC, CD8). Er vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, the methods include assessing expression of one or more markers on the surface of the modified cells or cells to be engineered. In one embodiment, the methods include assessing surface expression of TCR, MHC I, or CD3 (e.g., CD3ε), for example, by affinity-based detection methods such as by flow cytometry. In some aspects, where the method reveals surface expression of the antigen or other marker, the gene encoding the antigen or other marker is disrupted or expression otherwise repressed for example, using the methods described herein

### Isolation and Enrichment of Modified T Cells

In some embodiments, the method described herein further comprise isolating or enriching T cells comprising the first and/or the second nucleic acid. In some embodiments, the method described herein further comprises isolating or enriching endogenous MHC I-negative T cells from the modified T cell. In some embodiments, the method described herein further comprises isolating or enriching CD4⁺ and/or CD28⁺ T cells from the modified T cells. In some embodiments, the method described herein further comprises isolating or enriching modified T cells expressing the functional exogenous receptor described herein. In some embodiments, the isolation or enrichment of T cells comprises any combinations of the methods described herein.

In some embodiments, the isolation methods include the separation of different cell types based on the absence or presence in the cell of one or more specific molecules, such as surface markers, e.g., surface proteins, intracellular markers, or nucleic acid. In some embodiments, the selection marker is functional exogenous receptor and/or MHC I. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule. e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained, for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec. Auburn. Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of croelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5):355-376. In both cases, cells can be labeled with multiple markers, allowing for the isolation of well-defined T cell subsets at high purity. In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

### Gene-Editing of Endogenous Loci

In some embodiments, the endogenous loci of the T cell, such as endogenous TCR loci (e.g., TCRα, TCRβ), B2M (beta-2-microglobulin; can lead to deficiency in MHC Class I molecule expression and/or depletion of CD8⁺ T cells), CD38, and CS1, is modified by a gene-editing method, prior to or simultaneously with modifying the T cell to express one or more CARs or other functional exogenous receptors described herein.

In some embodiments, the modification of the endogenous loci is carried out by effecting a disruption in the gene, such as a knock-out, insertion, missense or frameshift mutation, such as a biallelic frameshift mutation, deletion of all or part of the gene, e.g., one or more exon or portion thereof, and/or knock-in. In some embodiments, such locus modification is performed using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

In some embodiments, the modification of endogenous loci is carried out using one or more DNA-binding nucleic acids, such as disruption via an RNA-guided endonuclease (RGEN), or other form of repression by another RNA-guided effector molecule.

For example, in some embodiments, the repression is carried out using clustered regularly interspaced short palindromic repeats (CRISPR) and CRISPR-associated (Cas) proteins. See Sander and Joung, Nature Biotechnology, 32 (4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a proto spacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to an eterologous effector domain such as a transcriptional repressor or activator, to affect gene expression.

In some embodiments, an endogenous locus of a T cell is modified by CRISPR/Cas system prior to modifying the T cell to express one or more functional exogenous receptors such as CARs. In some embodiments, an endogenous loci of a T cell is modified by CRISPR/Cas system simultaneously with modifying the T cell to express one or more functional exogenous receptors such as CARs described herein. In some embodiments, the nucleic acid(s) encoding the CRISPR/Cas system and the nucleic acid(s) encoding one or more functional exogenous receptors described herein are on the same vector, either optionally controlled by the same promoter or different promoters. In some embodiments, the nucleic acid(s) encoding the CRISPR/Cas system and the nucleic acid(s) encoding one or more functional exogenous receptors described herein described herein are on different vectors.

Many other techniques (such as knockout or knockdown techniques) are known in the art for deleting, inactivating or altering the function of genes, and the present disclosure includes these known techniques, such as nucleases based techniques or RNA based techniques (e.g., RNA interference (RNAi), small interfering RNA (siRNA) or short hairpin RNA (snRNA) based techniques).

In some embodiments, MHC I is knocked out or knocked down in the present T cells. In other embodiments, CD38 is knocked out or knocked down in the present T cells. In yet other embodiments. CS1 is knocked out our knocked down in the present T cells.

### Down Regulation of MHC I

Specifically, in certain embodiments of the various engineered immune cells (allogeneic T cells) provided herein, the cell surface expression of MHC I molecule is down regulated (or reduced).

The human MHC is called the HLA (Human Leukocyte Antigen). The MHC molecules are highly polymorphic. Allogeneic cells express MHC class I and MHC class II molecules, which may cause graft rejection when administered to an MHC-mismatched patient. Two major classes of MHC molecules, MHC class I and MHC class II molecules, determine the recognition of foreign cells by T-lymphocytes in a host.

MHC class I molecules are expressed on the surface of almost all nucleated cells, including immune cells and HSCs. The MHC class I molecule is a heterodimer comprising a highly polymorphic alpha heavy chain, which is non-covalently associated with a conserved light chain called beta-2 microglobulin (B2M). The heavy chains of HLA class I molecules are encoded by major class I HLA genes HLA-A. HLA-B and HLA-C, and minor class I HLA genes HLA-E, HLA-F and HLA-G. The conserved P2-microglobulin binds with a subunit encoded by a major or minor class I HLA gene to produce a functional HLA class I heterodimer on the surface of a cell. MHC class I molecules present endogenously synthesized peptides to host CD8⁺ T lymphocytes.

There are several proposed mechanisms for recognition of alloantigens on graft cells. Allo-MHC molecules can create many new pMHC complexes that can serve as ligands for various T cell clones. See, for example, Front Immunol. 13(3):184. The prevalence of either model in T cell allorecognition presumably depends upon the degree of heterogeneity (structural and/or conformational) between recipient and donor MHC molecules. Induction of the adaptive immune response to an allograft begins with recognition of alloantigen by recipient T cells occur through three main processes known as the direct, the indirect, and the semi-direct pathways of antigen presentation. In direct allorecognition, naive T cells located in lymph nodes can become activated through recognition of allogeneic MHC molecules displayed on donor passenger leukocytes. The direct T cell alloresponse is polyclonal in that it involves a large portion of the T cell repertoire (1-10%). In indirect allorecognition, T cells interact with donor peptides (derived from MHC and minor histocompatibility antigens) processed and presented by recipient APCs, thus indirectly activate allorecognition. This can be inhibited, for example, by expressing sICP47 in the cells. In semi-direct allorecognition, T cells can also recognize intact donor MHC molecules on recipient APCs (MHC molecules transferred from donor to recipient APCs).

More specifically, MHC class I H chain and B2M assemble with peptide in a multimeric complex with calreticulin, ERp57, tapasin, and the TAP heterodimer in the ER. Certain viral proteins retard MHC class I egress or induce its turnover, in some cases by ejection of the molecules from the ER into the cytoplasm. Peptides are provided by proteasomal cleavage of ubiquitinated cytosolic proteins and TAP transport into the ER, and both TAP and the proteasome are known targets for viral interference. Within the ER, the peptides are N-terminally trimmed by ER aminopeptidase associated with Ag presentation (ERAAP). Once peptide is bound, the complete MHC class I molecule is released from ER chaperones and proceeds through the Golgi. Via vesicular transport, the MHC class I molecule reaches the cell surface where it can present peptide to CTL (see J Immunol, 2003, 171:4473-4478; and PNAS, 2003, 102: 5144-5149).

After the arrival of MHC class I molecules at the cell surface, the presence of certain virus proteins can cause the endocytosis of MHC class I molecules. For example, HIV-1 Nef binds MHC class I on its cytoplasmic tail and escorts it from the cell membrane into the endosomal compartment. These MHC molecules are subsequently degraded, or they are transported into the trans-Golgi with the assistance of protein transport proteins like phosphofurin acidic cluster sorting protein-1, adaptor protein complexes, and phosphoinositide 3-kinase (see J biomed biotechnol, Volume 2011, Article ID 724607).

Other viruses, such as human CMV (HCMV) invests heavily in products able to interfere with MHC class I. The HCMV unique short (US) genes (US2, US3, US6, and US11) all assist HCMV in evading MHC class I presentation. HCMV encodes a unique long (UL) region protein, UL 18, which is an MHC class I homolog, capable of binding b2m and peptide.

Notably, Herpes Simplex Virus 1 (HSV-1) and HSV-2 encode a soluble cytoplasmic protein, ICP47, that associates with the peptide binding site formed by the C-terminal cytosolic domains of TAP1 and TAP2, thereby acting as a high-affinity competitor for peptide binding to the MHC class I molecules. See International Immunology, 9(19): 1115-1122.

Any known knockdown and knockout methods can be used herein to reduce the cell surface expression of MHC I molecule of the present engineered T cells including those described immediately above.

In addition, in certain embodiments, the modified therapeutic cells described herein express a simian ICP47 (sICP47) protein or a functional variant thereof for reducing MHC I expression on cell surface, for example, as described in Section 6 below as well as described in PCT/CN2020/090069, which content is incorporated herein by reference in its entirety.

Without being bound by theory, the active domain of sICP47 blocks peptide binding to TAP, as well as suppress peptide transport by TAP. sICP47 is a viral protein that plays a role in the inhibition of the host immune response, by blocking antigen processing and presentation in the host cells (see J Exp Med. 1997 May 5; 185(9): 1565-1572.). In general, ICP47 protein binds specifically to TAP (transporters associated with antigen processing) in the infected cells. The interactions between sICP47 and TAP blocks peptide binding and translocation by TAP, and subsequently inhibits loading of peptides onto MHC class I molecules. Without peptides bound, MHC I molecules are subjected to proteasomal degradation in the endoplasmic reticulum. Thus, infected cells are masked for immune recognition by cytotoxic T-lymphocytes. See, for example, Journal of virology 74.10 (2000): 4465-4473.

In some embodiments according to any one of the engineered T cells or methods described above, expression of the sICP47 protein or functional variant thereof downregulates cell surface expression of MHC molecules in the engineered T cell. In some embodiments, expression of the sICP47 protein or functional variant thereof downregulates cell surface expression of MHC molecules in the T cell by at least about any one of 10%, 20%, 30%, 40%, 50%, 60%, 70%, 80%, 90% or more, compared to a T cell that does not express the sICP47 protein or functional variant thereof.

In some embodiments, the sICP47 protein is derived from ICP47 of SA8. In some embodiments, the sICP47 protein comprises an active domain of ICP47 of SA8 or a functional variant thereof. In some embodiments, the sICP47 protein comprises full-length sICP47 of SA8 or a functional variant thereof. In some embodiments, the sICP47 protein is derived from ICP47 of Cercopithecine herpesvirus 16 (CeHV-16). In some embodiments, the sICP47 protein comprises an active domain of ICP47 of CeHV-16 or a functional variant thereof. In some embodiments, the sICP47 protein comprises full-length sICP47 of CeHV-16 or a functional variant thereof. In some embodiments, the sICP47 protein is derived from ICP47 of Cercopithecine herpesvirus 1 (CeHV-1). In some embodiments, the sICP47 protein comprises an active domain of ICP47 of CeHV-1 or a functional variant thereof. In some embodiments, the sICP47 protein comprises full-length sICP47 of CeHV-1 or a functional variant thereof. In some embodiments, the sICP47 protein is derived from ICP47 of Macacine alphaherpesvirus 1. In some embodiments, the sICP47 protein comprises an active domain of ICP47 of Macacine alphaherpesvirus 1 or a functional variant thereof. In some embodiments, the sICP47 protein is derived from ICP47 of Pappine alphaherpesvirus 2. In some embodiments, the sICP47 protein comprises an active domain of ICP47 of Pappine alphaherpesvirus 2 or a functional variant thereof.

In some embodiments, the sICP47 protein described in PCT/CN2020/090069 is expressed in the present engineered immune cells (e.g., CAR-T cells provided herein).

In some specific embodiments, a MHC I knockdown molecule described in Section 6 below is expressed in the present engineered T cell, such as SEQ ID NO: 10, SEQ ID NO; 11, SEQ ID NO: 12, SEQ ID NO: 13, SEQ ID NO: 14 and SEQ ID NO: 15, or variants thereof.

5.7. Pharmaceutical Compositions

In one aspect, the present disclosure further provides pharmaceutical compositions comprising an engineered immune cell (e.g., an engineered T cell) or a therapeutic agent such as antibodies of the present disclosure. In some embodiments, a pharmaceutical composition comprises a therapeutically effective amount of the engineered T cell of the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, omithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability. Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively. or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

5.8. Methods and Uses

In another aspect, provided herein are methods for using and uses of the engineered immune cells (e.g., an engineered T cell) provided herein. Such methods and uses include therapeutic methods and uses, for example, involving administration of the cells, or compositions containing the same, to a subject having a disease or disorder. In some embodiments, the cell is administered in an effective amount to effect treatment of the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present CAR-T cell therapies are used for treating solid tumor cancer. In other embodiments, the present CAR-T cell therapies are used for treating blood cancer. In other embodiments, the disease or disorder is an autoimmune and inflammatory disease.

In some embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments, the disease or disorder is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In other embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome. AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemo lytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale. Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpura, hepatitis A, hepatitis B, hepatitis C, HIV infection/HIV neuropathy, Hodgkin's disease, hypoparathyroidism. Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, mvasthenia gravis, microscopic vasculitis of the kidneys, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis. Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis. Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Th1-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, Yersinia and Salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

In some embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Cytokines and prostaglandins control the inflammatory response, and are released in an ordered and self-limiting cascade into the blood or affected tissues. This release of cytokines and prostaglandins increases the blood flow to the area of injury or infection, and may result in redness and warmth. Some of these chemicals cause a leak of fluid into the tissues, resulting in swelling. This protective process may stimulate nerves and cause pain. These changes, when occurring for a limited period in the relevant area, work to the benefit of the body.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as psoriasis, rheumatoid arthritis, osteoarthritis, psoriatic arthritis, Crohn's disease, asthma, allergies or inflammatory bowel disease, are characterized by chronic inflammation. Inflammatory diseases such as arthritis, related arthritic conditions (e.g., osteoarthritis, rheumatoid arthritis, and psoriatic arthritis), inflammatory bowel disease (e.g., Crohn's disease and ulcerative colitis), sepsis, psoriasis, atopic dermatitis, contact dermatitis, and chronic obstructive pulmonary disease, chronic inflammatory pulmonary diseases are also prevalent and problematic ailments.

In some embodiments, the methods include adoptive cell therapy, whereby genetically engineered cells are administered to a subject. Such administration can promote activation of the cells (e.g., T cell activation), such that the cells of the disease or disorder are targeted for destruction.

In some embodiments, the methods include administration of the cells or a composition containing the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive T cell therapy. In some embodiments, the cells or compositions are administered to the subject, such as a subject having or at risk for the disease or disorder. In some embodiments, the methods thereby treat, e.g., ameliorate one or more symptom of the disease or disorder.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, either simultaneously or sequentially in any order. In some embodiments, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

5.9. Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein. A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Pre-Treatments to Deplete Host NK Cells

In this example, exemplary therapeutic antibodies (e.g., anti-CD38 antibodies such as Daratumumab (Johnson & Johnson)) were used to deplete host NK cells prior to infusion of CAR-T cells. Exemplary sequences in the present studies are shown in Table 2 below.

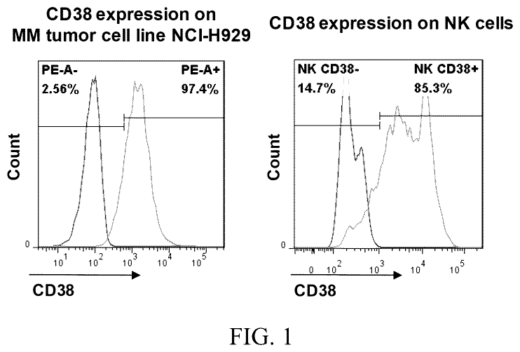

CD38 was shown to overexpress in multiple myeloma cells and activated NK cells. Specifically, by FACS staining analysis, CD38 was confirmed to be highly expressed on multiple myeloma (MM) tumor cell line NCI-H929 cells and activated NK cells. As shown in FIG. 1, MM tumor cell line NCI-H929 cells and activated NK cells were stained with anti-CD38 antibody, the NCI-H929 cells showed 97.4% CD38⁺ population, and active NK showed 85.3% CD38⁺ population. Both the MM tumor cells and NK cells can be recognized by anti-CD38 antibody (such as Daratumumab, Isatuximab, etc.) and eliminated by Antibody-Dependent Cell-Mediated Cytotoxicity (ADCC), Complement Dependent Cytotoxicity (CDC) and antibody-dependent cell-mediated phagocytosis (ADCP).

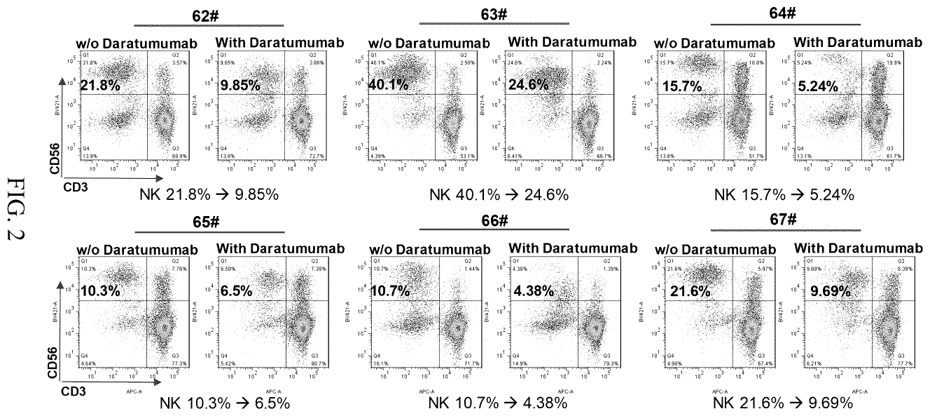

As shown in FIG. 2, in vitro treatment of PBMC with Daratumumab decreased the percentage of NK cells in the population. Specifically, PBMC cells from six donors were randomly selected. The cells were treated with anti-CD38 antibody (i.e., Daratumumab) overnight, and stained with CD56 antibody and CD3 antibody. Then flow cytometry analysis was performed. The results indicate that the NK % population decreased in all of the six donors when treated with anti-CD38 antibody (Daratumumab).

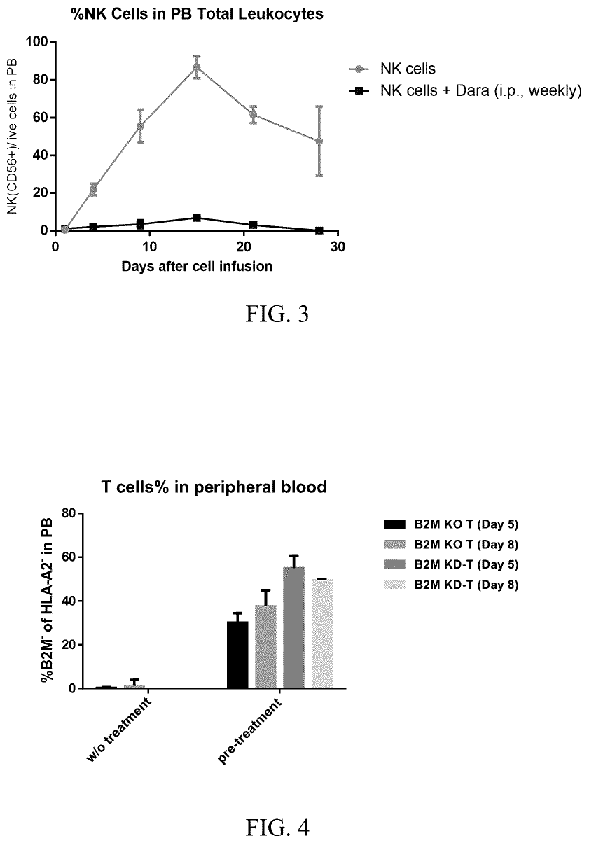

NK cells were administered intravenously into NCG mice (NOD_Prkdcem26Cd52/NjuCrl) at day 0, day 2 and day 4. Three mice were treated with anti-CD38 antibody (Daratumumab) weekly at dose of 0.5 μg/mice, and other four mice were dosed with HBSS buffer simultaneously. By monitoring the CD56⁺ cells in mice peripheral blood, it showed that Daratumumab continuously cleared NK cells in vivo (see FIG. 3).

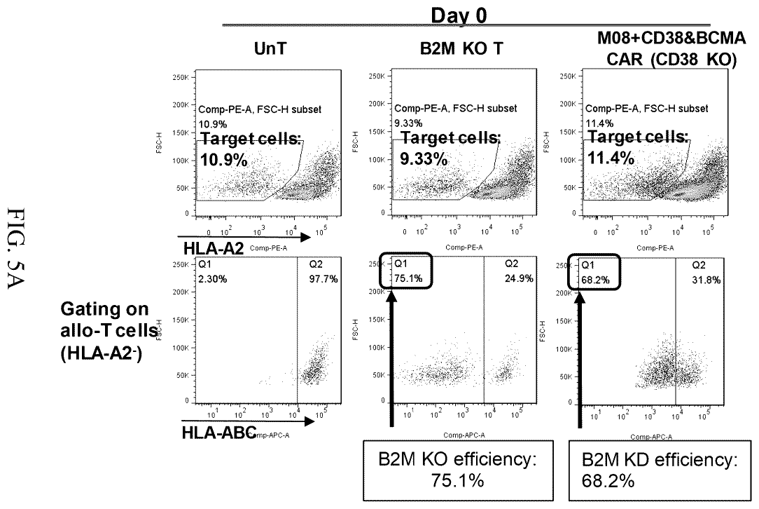

Next, the effects of in vivo treatment with Daratumumab on the survival of MHC I KD/KO T cells were tested. Specifically, a host verse graft in vivo model was assessed by NCG mouse (NOD_Prkdcem26Cd52/NjuCrl). At day 0, 10M of NK cells (HLA-A2⁺ donor) were administered intravenously into mice, with or without anti-CD38 antibody (Daratumumab) infusion (dose of 0.5 ug/mice i.p., weekly). 5M B2M KO T cells or 5M B2M KD T cells (HLA-A2⁻ donor) were administered intravenously into mice separately at day 3. B2M KO T cells were generated by CRISPR KO technology, and KO efficiency was 89%. B2M KD T cells were transduced with MHC I KD molecule (see SEQ ID NOs: 10 to 15), and the KD efficiency was 44%. The percentage of HLA-A2⁻ cells in peripheral blood at day 5 and day 8 after T cells infusion was analyzed. Groups without Daratumumab treatment showed almost no B2M KO T cells left, meaning that the host NK cells rejected the graft T cells. In the groups with Daratumumab treatment, there were still B2M KO T cells and B2M KD T cells as shown in FIG. 4. These results indicate that pre-treatment with a therapeutic agent for depleting host NK cells increases the survival of the infused engineered T cells. We chose a B2M KD T cell which was transduced with MHC I KD molecule (M08, SEQ ID NO: 15) for further evaluation below.

TABLE 2
Exemplary Sequences

| SEQ ID NO. | Name | Sequence |
|---|---|---|
| SEQ ID NO: 1 | VH of | EVQLLESGGGLVQPGGSLRLSCAVSGFTENSFAMSWVRQAPG |
| | Daratumumab | KGLEWVSAISGSGGGTYYADSVKGRFTISRDNSKNTLYLQMN |
| | (anti-CD38 | SLRAEDTAVYFCAKDKILWFGEPVEDYWGQGTLVTVSS |
| | antibody) | |
| SEQ ID NO: 2 | VL of | EIVLTQSPATLSLSPGERATLSCRASQSVSSYLAWYQQKPGQA |
| | Daratumumab | PRLLIYDASNRATGIPARFSGSGSGTDFTLTISSLEPEDFAVYYC |
| | (anti-CD38 | QQRSNWPPTFGQGTKVEIK |
| | antibody) | |
| SEQ ID NO: 3 | VH of Isatuximab | QVQLVQSGAEVAKPGTSVKLSCKASGYTFTDYWMQWVKQR |
| | (anti-CD38 | PGQGLEWIGTIYPGDGDTGYAQKFQGKATLTADKSSKTVYM |
| | antibody) | HLSSLASEDSAVYYCARGDYYGSNSLDYWGQGTSVTVSS |
| SEQ ID NO: 4 | VL of Isatuximab | DIVMTQSHLSMSTSLGDPVSITCKASQDvsTVVAWYQQKPGQS |
| | (anti-CD38 | PRRLIY&AsYRYIGVPDRFTGSGAGTDFTFTISSVQAEDLAVYY |
| | antibody) | CQQHYSPPYTFGGGTKLEIKRT |
| SEQ ID NO: 5 | VH of elotuzumab | SEVQLVESGGGLVQPGGSLRLSCAASGFDFSRYWMSWVRQA |
| | (anti-CSI | PGKGLEWIGEINPDSSTINYAPSLKDKFIISRDNAKNSLYLQMN |
| | antibody) | SLRAEDTAVYYCARPDGNYWYFDVWGQGTLVTVSS |
| SEQ ID NO: 6 | VL of elotuzumab | DIQMTQSPSSLSASVGDRVTITCKASQDVGIAVAWYQQKPGK |
| | (anti-CSI | VPKLLIYWASTRHTGVPDRFSGSGSGTDFTLTISSLQPEDVATY |
| | antibody) | YCQQYSSYPYTFGQGTKVEIK |
| SEQ ID NO: 7 | anti-BCMAL sdAb | EVQLVESGGGLVQAGGSLRLSCAASGRTFTMGWFRQAPGKE |
| | | REFVAAISLSPTLAYYAESVKGRFTISRDNAKNTVVLQMNSLK |
| | | PEDTALYYCAADRKSVMSIRPDYWGQGTQVTVSS |
| SEQ ID NO: 8 | anti-BCMA2 sdAb | QVQLEESGGGSVQAGGSLRLSCAYTYSTYSNYYMGWFREAP |
| | | GKARTSVAIISSDTTITYKDAVKGRFTISKDNAKNTLYLQMNS |
| | | LKPEDSAMYRCAAWTSDWSVAYWGQGTQVTVSS |
| SEQ ID NO: 9 | anti-BCMA3 sdAb | AVQLVESGGGLVQAGDSLRLTCTASGRAFSTYFMAWFRQAP |
| | | GKEREFVAGIAWSGGSTAYADSVKGRFTISRDNAKNTVYLQM |
| | | NSLKSEDTAVYYCASRGIEVEEFGAWGQGTQVTVSS |
| SEQ ID NO: 10 | K5 (HHV8P) | MASKDVEEGVEGPICWICREEVGNEGIHPCACTGELDVVHPQ |
| | | CLSTWLTVSRNTACQMCRVIYRTRTQWRSRLNLWPEMERQEI |
| | | FELFLLMSVVVAGLVGVALCTWTLLVILTAPAGTFSPGAVLGF |
| | | LCFFGFYQIFIVFAFGGICRVSGTVRALYAANNTRVTVLPYRRP |
| | | RRPTANEDNIELTVLVGPAGGTDEEPTDESSEGDVASGDKERD |
| | | GSSGDEPDGGPNDRAGLRGTARTDLCAPTKKPVRKNHPKNN |
| | | G |
| SEQ ID NO: 11 | US6 (HHV-5) | MDLLIRLGFLLMCALPTPGERSSRDPKTLLSLSPRQQACVPRT |
| | | KSHRPVCYNDTGDCTDADDSWKQLGEDFAHQCLQAAKKRPK |
| | | THKSRPNDRNLEGRLTCQRVRRLLPCDLDIHPSHRLLTLMNNC |
| | | VCDGAVWNAFRLIERHGFFAVTLYLCCGITLLVVILALLCSITY |
| | | ESTGRGIRRCGS |
| SEQ ID NO: 12 | ICP47 (HHV-1) | MSWALEMADTFLDTMRVGPRTYADVRDEINKRGREDREAAR |
| | | TAVHDPERPLLRSPGLLPEIAPNASLGVAHRRTGGTVTDSPRN |
| | | PVTR |
| SEQ ID NO: 13 | SICP47 of CeHV- | MSSLYLAEVDAFLQSPRTRHRTCADLRRELDAYADEERREAA |
| | 16 | KAIAHPDRPLLAPPSAPPDRSRPAPRGTAHPPAASP |
| SEQ ID NO: 14 | SICP47 of CeHV-1 | MSSRYLAAVDDYLHHPSPRYQAHVDLRRELRAYADEERREA |
| | | ARAIAHPERPLLPPPATQAAPPQPSTREAAHPSAPTAASS |
| SEQ ID NO: 15 | SICP47 of SA8 | MSSLYLATVDAFLRNPHTRHRTCADLRRELDAYADEERREAA |
| | | KAIAHPDRPLLAPPSAPPNHSHLAARETAPPPAATP |
| SEQ ID NO: 16 | GS linker1 | GGGGS |
| SEQ ID NO: 17 | GS linker2 | GGGGSGGGGS |
| SEQ ID NO: 18 | GS linker3 | GGGGSGGGGSGGGGS |
| SEQ ID NO: 19 | CD8a leading | MALPVTALLLPLALLLHAARP |
| | peptide | |
| SEQ ID NO: 20 | CD&a hinge | TTTPAPRPPTPAPTIASQPLSLRPEACRPAAGGAVHTRGLDFAC |
| | | D |
| SEQ ID NO: 21 | CD8a | IYIWAPLAGTCGVLLLSLVITLYC |
| | transmembrane | |
| SEQ ID NO: 22 | CD4 leading | MNRGVPFRHLLLVLQLALLPAATQG |
| | peptide | |
| SEQ ID NO: 23 | CD4 | SGQVLLESNIKVLPTWSTPVQPMALIVLGGVAGLLLFIGLGIFF |
| | transmembrane | |
| SEQ ID NO: 24 | intracellular | RVKFSRSADAPAYQQGQNQLYNELNLGRREEYDVLDKRRGR |
| | domain of CD3 | DPEMGGKPRRKNPQEGLYNELQKDKMAEAYSEIGMKGERRR |
| | zeta | GKGHDGLYQGLSTATKDTYDALHMQALPPR |
| SEQ ID NO: 25 | intracellular | KRGRKKLLYIFKQPFMRPVQTTQEEDGCSCRFPEEEEGGCEL |
| | domain of 4-1BB | |
| SEQ ID NO: 26 | CD3 epsilon | MQSGTHWRVLGLCLLSVGVWGQ |
| | leading peptide | |
| SEQ ID NO: 27 | CD3 epsilon | DGNEEMGGITQTPYKVSISGTTVILTCPQYPGSEILWQHNDKNI |
| | | GGDEDDKNIGSDEDHLSLKEFSELEQSGYYVCYPRGSKPEDA |
| | | NFYLYLRARVCENCMEMDVMSVATIVIVDICITGGLLLLVYY |
| | | WSKNRKAKAKPVTRGAGAGGRQRGQNKERPPPVPNPDYEPI |
| | | RKGQRDLYSGLNQRRI |
| SEQ ID NO: 28 | VH of anti-IL-T2 | QVQLQQSGAELVKPGASVRLSCKASGYTFTAHTIHWVKQRSG |
| | antibody | QGLEWIGWLYPGSGSIKYNEKFKDKATLTADKSSSTVYMELS |
| | | RLTSEDSAVYFCARHTNWDFDYWGQGTTLTVSS |
| SEQ ID NO: 29 | VL of anti-IL-T2 | NIVLTQSPASLAVSLGQRATISCKASQSVDYGGASYMNWYQQ |
| | antibody | KPGQPPKLLIYAASNLESGIPARFSGSGSGTDLTLNIHPVEEED |
| | | AAMYYCQQSNEEPWTFGGGTKLEIK |
| SEQ ID NO: 30 | VH of anti-CD137 | EVQLVQSGAEVKKPGESLRISCKGSGYSFSTYWISWVRQMPG |
| | antibody | KGLEWMGKIYPGDSYTNYSPSFQGQVTISADKSISTAYLQWSS |
| | | LKASDTAMYYCARGYGIFDYWGQGTLVTVSS |
| SEQ ID NO: 31 | VL of anti-CD137 | SYELTQPPSVSVSPGQTASITCSGDNIGDQYAHWYQQKPGQSP |
| | antibody | VLVIYQDKNRPSGIPERFSGSNSGNTATLTISGTQAMDEADYY |
| | | CATYTGFGSLAVFGGGTKLTVL |
| SEQ ID NO: 32 | VH of anti- | QVQLVQSGAEVKKPGASVKVSCKASGYTFTSYWMNWVRQA |
| | NKG2A antibody | PGQGLEWMGRIDPYDSETHYAQKLQGRVTMTTDTSTSTAYM |
| | | ELRSLRSDDTAVYYCARGGYDFDVGTLYWFFDVWGQGTTVT |
| | | VSS |
| SEQ ID NO: 33 | VL of anti-NKG2A | DIQMTQSPSSLSASVGDRVTITCRASENIYSYLAWYQQKPGKA |
| | antibody | PKLLIYNAKTLAEGVPSRFSGSGSGTDFTLTISSLQPEDFATYY |
| | | CQHHYGTPRTFGGGTKVEIK |
| SEQ ID NO: 34 | VH of anti- | QVHLQESGPGLVKPSETLSLTCTVSDDSISSYYWSWIRQPPGK |
| | NKG2D-1 | GLEWIGHISYSGSANYNPSLKSRVTISVDTSKNQFSLKLSSVTA |
| | antibody | ADTAVYYCANWDDAFNIWGQGTMVTVSS |
| SEQ ID NO: 35 | VL of anti- | EIVLTQSPGTLSLSPGERATLSCRASQSVSSSYLAWYQQKPGQ |
| | NKG2D-1 | APRLLIYGASSRATGIPDRFSGSGSGTDFTLTISRLEPEDFAVYY |
| | antibody | CQQYGSSPWTFGQGTKVEIK |
| SEQ ID NO: 36 | VH of anti- | EVQLVQSGAEVKEPGESLKISCKNSGYSFTNYWVGWVRQMP |
| | NKG2D-2 | GKGLEWMGIIYPGDSDTRYSPSFQGQVTISADKSINTAYLQWS |
| | antibody | SLKASDTAMYYCGRLTMFRGIIIGYFDYWGQGTLVTVSS |
| SEQ ID NO: 37 | VL of anti- | EIVLTQSPATLSLSPGERATLSCRASQSVSSYLAWYQQKPGQA |
| | NKG2D-2 | PRLLIYDASNRATGIPARFSGSGSGTDFTLTISSLEPEDFAVYYC |
| | antibody | QQRSNWPWTFGQGTKVEIK |
| SEQ ID NO: 38 | VH of anti-CD16 | EVQLVESGGGVVRPGGSLRLSCAASGFTFDDYGMSWVRQAP |
| | antibody | GKGLEWVSGINWNGGSTGYADSVKGRFTISRDNAKNSLYLQ |
| | | MNSLRAEDTAVYYCARGRSLLFDYWGQGTLVTVSR |
| SEQ ID NO: 39 | VL of anti-CD16 | SELTQDPAVSVALGQTVRITCQGDSLRSYYASWYQQKPGQAP |
| | antibody | VLVIYGKNNRPSGIPDRFSGSSSGNTASLTITGAQAEDEADYYC |
| | | NSRDSSGNHVVFGGGTKLTVL |
| SEQ ID NO: 40 | VH of anti-CD16a | QVQLVQSGAEVKKPGESLKVSCKASGYTFTSYYMHWVRQAP |
| | antibody | GQGLEWMGIINPSGGSTSYAQKFQGRVTMTRDTSTSTVYMEL |
| | | SSLRSEDTAVYYCARGSAYYYDFADYWGQGTLVTVSS |
| SEQ ID NO: 41 | VL of anti-CD16a | SYVLTQPSSVSVAPGQTATISCGGHNIGSKNVHWYQQRPGQSP |
| | antibody | VLVIYQDNKRPSGIPERFSGSNSGNTATLTISGTQAMDEADYY |
| | | CQVWDNYSVLFGGGTKLTVL |
| SEQ ID NO: 42 | VH of anti-KIR | QIQLVQSGSELKKPGASVKVSCKASGYTFTTAGMQWVRQAP |
| | antibody | GQGLEWIGWINSHSGVPKYAEDFKGRFVFSLDTSVSTAYLQIS |
| | | SLKAEDTAVYFCARGGDEGVMDYWGQGTTVTVSS |
| SEQ ID NO: 43 | VL of anti-KIR | DIQMTQSPSFLSASVGDRVTITCKASQDVSTAVAWYQQKPGQ |
| | antibody | PPKLLIYWTSTRHTGVPDRFSGSGSGTDYTLTISSLQAEDVAV |
| | | YYCQQHYSTPWTFGGGTKVEIK |
| SEQ ID NO: 44 | VH of anti-CD56 | QVQLVESGGGVVQPGRSLRLSCAASGFTFSSFGMHWVRQAPG |
| | antibody | KGLEWVAYISSGSFTIYYADSVKGRFTISRDNSKNTLYLQMNS |
| | | LRAEDTAVYYCARMRKGYAMDYWGQGTLVTVSS |
| SEQ ID NO: 45 | VL of anti-CD56 | DVVMTQSPLSLPVTLGQPASISCRSSQIIIHSDGNTYLEWFQQR |
| | antibody | PGQSPRRLIYKVSNRFSGVPDRFSGSGSGTDFTLKISRVEAEDV |
| | | GVYYCFQGSHVPHTFGQGTKVEIK |
| SEQ ID NO: 46 | VH of anti-CD226- | EVQLLESGGGLVQPGGSLRLSCAASGFTFSSYAMSWVRQAPG |
| | 1 antibody | KGLEWVSAISGSGGSTYYADSVKGRFTISRDNSKNTLYLQMN |
| | | SLRAEDTAVYYCARDWVTTGPFAFDLWGQGTMVTVSS |
| SEQ ID NO: 47 | VL of anti-CD226- | DIQMTQSPSSLSASVGDRVTITCRASQSISSYLNWYQQKPGKA |
| | 1 antibody | PKLLIYAASSLQSGVPSRFSGSGSGTDFTLTISSLQPEDFATYYC |
| | | QQYYTLPFTFGGGTKVEIK |
| SEQ ID NO: 48 | VH of anti-CD226- | EVQLLESGGGLVQPGGSLRLSCAASGFTFSSYAMSWVRQAPG |
| | 2 antibody | KGLEWVSAISGSGGSTYYADSVKGRFTISRDNSKNTLYLQMN |
| | | SLRAEDTAVYYCARDSSGYYFDYWGQGTLVTVSS |
| SEQ ID NO: 49 | VL of anti-CD226- | EIVMTQSPATLSVSPGERATLSCRASQSVSSNLAWYQQKPGQ |
| | 2 antibody | APRLLIYGASTRATGIPARFSGSGSGTEFTLTISSLQSEDFAVYY |
| | | CQQYNKWPLFTFGGGTKVEIK |
| SEQ ID NO: 50 | VH of anti-CD25-1 | QVQLVQSGAEVKKPGSSVKVSCKASGYTFTSYRMHWVRQAP |
| | antibody | GQGLEWIGYINPSTGYTEYNQKFKDKATITADESTNTAYMELS |
| | | SLRSEDTAVYYCARGGGVFDYWGQGTLVTVSS |
| SEQ ID NO: 51 | VL of anti-CD25-1 | DIQMTQSPSTLSASVGDRVTITCSASSSISYMHWYQQKPGKAP |
| | antibody | KLLIYTTSNLASGVPARFSGSGSGTEFTLTISSLQPDDFATYYC |
| | | HQRSTYPLTFGQGTKVEVKR |
| SEQ ID NO: 52 | VH of anti-CD25-2 | QVQLVQSGAEVKKPGSSVKVSCKASGGTFSRYIINWVRQAPG |
| | antibody | QGLEWMGRIIPILGVENYAQKFQGRVTITADKSTSTAYMELSS |
| | | LRSEDTAVYYCARKDWFDYWGQGTLVTVSS |
| SEQ ID NO: 53 | VL of anti-CD25-2 | EIVLTQSPGTLSLSPGERATLSCRASQSVSSYLAWYQQKPGQA |
| | antibody | PRLLIYGASSRATGIPDRFSGSGSGTDFTLTISRLEPEDFAVYYC |
| | | QQYGSSPLTFGGGTKVEIK |
| SEQ ID NO: 54 | VH of anti-CD83 | QVQLKESGPGLVKPSQSLSLTCSVTGFSITTGGYWWTWIRQFP |
| | antibody | GQKLEWMGYIFSSGNTNYNPSIKSRISITRDTSKNQFFLQLNSV |
| | | TTEGDTARYYCARAYGKLGFDYWGQGTLVTVSS |
| SEQ ID NO: 55 | VL of anti-CD83 | QPVLTQSPSASASLGNSVKITCTLSSQHSTYTIGWYQQHPDKA |
| | antibody | PKYVMYVNSDGSHSKGDGIPDRFSGSSSGAHRYLSISNIQPED |
| | | EADYFCGSSDSSGYVFGSGTQLTVL |
| SEQ ID NO: 56 | VH of anti-KLRGI | EVQLVQSGAEVKKPGASVKVSCKASGYTFTDHNMHWVKQA |
| | antibody | TGQGLEWFGFINPNTGVTRYNQKFQGRVTLTINKAISTAYLEL |
| | | SSLRSEDTAVYYCARDYYGSAWFAYWGQGTLVTVSS |
| SEQ ID NO: 57 | VL of anti-KLRGI | DIVMTQSPDSLAVSLGERATINCKSSQTLLYSSDQKNYLAWY |
| | antibody | QQKPGQPPKLLIYWASTRESGVPDRFSGSGSGTDFTLTISSLQA |
| | | EDVAVYYCQQYYNYPTFGGGTKV |
| SEQ ID NO: 58 | VH of anti-CD70 | QVQLVQSGAEVKKPGASVKVSCKASGYTFTNYGMNWVRQA |
| | antibody | PGQGLKWMGWINTYTGEPTYADAFKGRVTMTRDTSISTAYM |
| | | ELSRLRSDDTAVYYCARDYGDYGMDYWGQGTTVTVSS |
| SEQ ID NO: 59 | VL of anti-CD70 | DIVMTQSPDSLAVSLGERATINCRASKSVSTSGYSFMHWYQQ |
| | antibody | KPGQPPKLLIYLASNLESGVPDRFSGSGSGTDFTLTISSLQAED |
| | | VAVYYCQHSREVPWTFGQGTKVEIK |
| SEQ ID NO: 60 | VH of anti-CD30 | QIQLQQSGPEVVKPGASVKISCKASGYTFTDYYITWVKQKPGQ |
| | antibody | GLEWIGWIYPGSGNTKYNEKFKGKATLTVDTSSSTAFMQLSSL |
| | | TSEDTAVYFCANYGNYWFAYWGQGTQVTVSAASTKGPSVFP |
| | | LAPSSKSTSGGTAALGCLVKDYFPEPVTVSWNS |
| SEQ ID NO: 61 | VL of anti-CD30 | DIVLTQSPASLAVSLGQRATISCKASQSVDFDGDSYMNWYQQ |
| | antibody | KPGQPPKVLIYAASNLESGIPARFSGSGSGTDFTLNIHPVEEED |
| | | AATYYCQQSNEDPWTFGGGTKLEIK |
| SEQ ID NO: 62 | VH of anti-CD229 | DIQMTQSPSSLSASVGDRVTITCPvASQSIGSSLHWYQQKPGKA |
| | antibody | PKFLIYDASSLESGVPSPvFSGSGSGTEFTLTISSLQPDDFATYY |
| | | CQQYNSYPLTFGGGTKLEIK |
| SEQ ID NO: 63 | VL of anti-CD229 | QITLKESGPTLVKPTQTLTLTCTFSGFSLSTSGVGVGWIRQPPG |
| | antibody | KALEWLALIYWNDDKRYSPSLKSRLTIAKDTSKNQVVLTMTN |
| | | MDPVDTATYYCARMGWNDPHMVDYWGQGTLVTVSS |
| SEQ ID NO: 64 | VH of anti-NCR3 | EIQLLESGGGLVQPGGSLRLSCAVSGFSITTTGYLIWNWVRQA |
| | antibody | PGKGLEWVGYIYSSGSTSYNPSLKSRFTISRDTSKNTFYLQMN |
| | | SLRAEDTAVYYCARGDWHYFDYWGQGTMVTVSS |
| SEQ ID NO: 65 | VL of anti-NCR3 | DSVTTQSPLSLPVTLGQPASISCSGEKLSDKYVHWYQQRPGQS |
| | antibody | PRMLIYENDRRPSGVPDRFSGSNSGNDATLKISRVEAEDVGVY |
| | | FCQFWDSTNSAVFGGGTKVEIK |

### 6.2. Example 2—Engineered T Cells Comprising a NK Cell Depleting Domain

Exemplary NK cell targeting CARs were constructed and tested in this example. More specifically, T cells comprising an anti-CD38 CAR and an anti-BCMA CAR were constructed. Exemplary sequences are shown in Table 2 above.

6.2.1. CD38&BCMA CAR-T Cells (with MHC I KD Molecule) Demonstrated Better Persistence than B2M KO T Cells and Wild Type T Cells

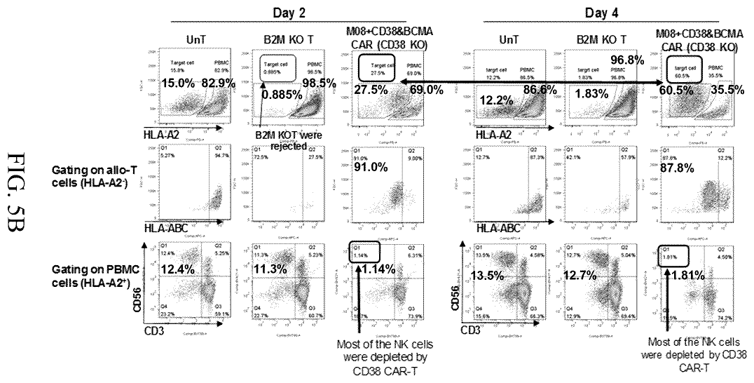

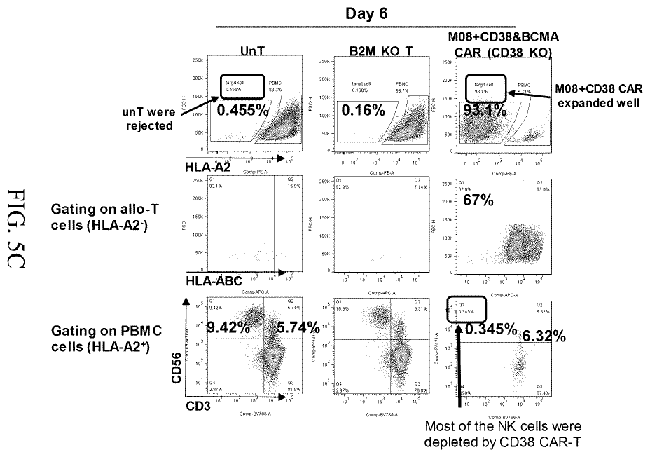

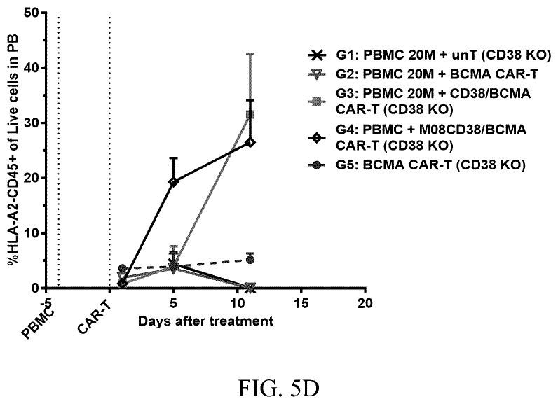

A MLR assay was conducted with PBMCs (HLA-A2⁺ donor) and allogeneic T cells (HLLA-A2⁻ donor). The ratio of PBMCs and graft T cells was 9:1. Several replicate wells were seeded for FACS staining at day 0, day 2, day 4 and day 6. At day 0, FACS analysis was performed to confirm the PBMCs:T ratios. As showed in FIG. 5A, the HLA-A2+ cells was 10.9% for unT, 9.33% for B2M KO T and 11.4% for M08+CD38&BCMA CAR-T (CD38 KO). CD38&BCMA CAR-T cells (CD38 KO) were dual CAR-T cells targeting both CD38 and BCMA, and CD38 was knocked-out in these T cells. M08 as used herein was a code for MHC I knockdown molecule.

CD38 was knocked out on primary T cells by CRISPR/Cas9 genome editing. Briefly, on days 1-2 of T cells activation, the T cells were collected by centrifugation at 200 g for 10 min. Cells were washed with 2 ml of DPBS and re-suspended for determination of cell density. The cell density was adjusted to 1×10⁶-4×10⁶ per 5 ml in a 15 ml centrifuge tube, followed by centrifugation at 200 g for 10 min. The supernatant was removed after centrifugation. Cas9 solution (made by GenScript) and RNP complex solution (premixed) were added to the cell, and the mixture was incubated for 20 min at room temperature. The solution for electroporation was prepared as suggested by the kit manual (Human T cell NUCLEOFECTOR™ kit). The electroporation solution was gently added to the cells and incubated for 10 min at room temperature. After incubation, the cell suspension was transferred into an electric rotor, and subjected to the electroporation procedure. After electroporation, 2 ml of pre-warmed medium was added to the cells. The cells were then incubated at 37° C. and 5% C02.

As shown in FIG. 5B for the FACS analysis data of day 2 and day 4, B2M KO T cells were almost rejected by allo-PBMCs cells at day 2 (from 9.33% to 0.885%). In contrast. M08+CD38&BCMA CAR-T cells expanded well, and from day 2 to day 4, the HLA-A2− cells % expended from 27.5% to 60.5%. When gating on HLA-A2+ cells (host PBMCs), in the wells of co-culturing PBMC and CD38&BCMA CAR-T (CD38 KO), host NK cells were almost depleted.

FACS analysis data of day 6 are shown in FIG. 5C. As shown, wild type T cells were almost rejected by allo-PBMCs cells at day 6 (0.455%) and B2M KO T cells were rejected completely by allo-PBMCs cells at day 6 (0.16%), whereas M08+CD38&BCMA CAR-T (CD38 KO) cells expanded well (93.1%). Therefore, compared to unT and B2M KO T cells, M08+CD38&BCMA CAR-T (CD38 KO) showed best persistence in MLR assay.

FIG. 5D shows the in vivo persistency of different products in the presence of allogeneic PBMCs in immunodeficient mouse model to mimic host rejection of allogeneic graft cells. The grafted unT cells (CD38 KO) and BCMA CAR-T cells were rejected by allogeneic PBMCs on day 11. Percentage of grafted unT cells and BCMA CAR-T cells in peripheral blood were 0.06% and 0.03%, respectively (Group 1&2). CD38&BCMA CAR-T (CD38 KO) cells and M08+CD38&BCMA CAR-T (CD38 KO) cells persisted well in the same setting. Percentage of grafted CD38&BCMA CAR-T (CD38 KO) cells and M08+CD38&BCMA CAR-T (CD38 KO) cells in peripheral blood were 31.52% and 26.48%, respectively on day 11 (Group 3&4). M08+CD38&BCMA CAR-T (CD38 KO) cells showed better persistence at earlier stage in comparison with CD38&BCMA CAR-T (CD38 KO) cells due to M08's help on escaping rejection from allogeneic T cells within PBMCs.

6.2.2. CD38&BCMA CAR-T Cells (with MHC I KD Molecule) Demonstrated Better Persistence than Anti-CD38 CAR-T Cells and B2M KO T Cells

A MLR assay was then conducted with PBMCs (HLA-A2⁻ donor) and allogeneic T cells (HLA-A2⁺ donor), and the ratio of PBMCs and graft T cells was 4:1. Several replicate wells were seeded for FACS staining at day 0, day 2, day 4, day 6 and day 8.

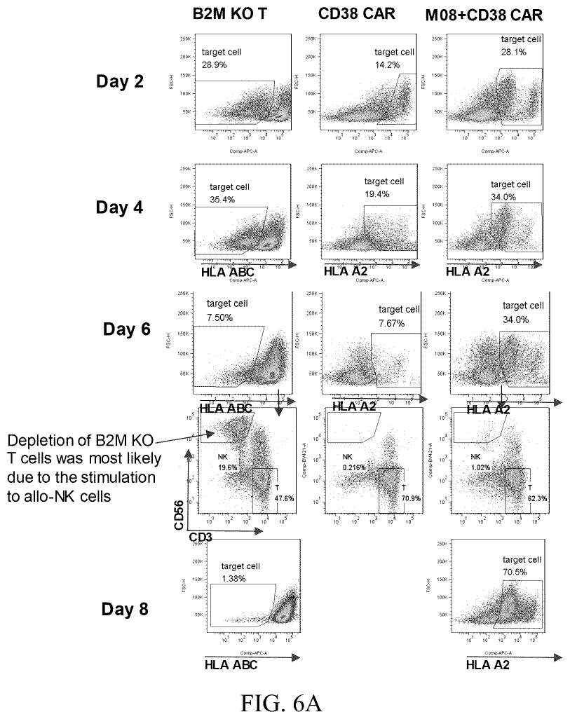

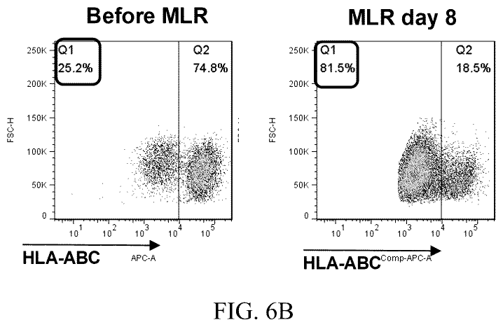

As shown in FIG. 6A, depletion of B2M KO T cells was observed at day 6, and it was most likely that B2M KO T cells (MHC-1 KO) stimulated the allo-NK cells and were killed by allo-NK cells. At day 6, CD38 CAR-T cells were also rejected by allo-PBMC cells (from day 2 to day 6: 14.2% to 7.67%), and CD38 CAR-T cell was almost not detectable at day 8. Though CD38 CAR-T cells killed the host NK cells (most of the NK cells were depleted by CD38 CAR-T), the remaining T cells still could reject the CD38 CAR-T cells. In contrast. M08+CD38&BCMA CAR-T cells expanded to 70.5% at day 8. Therefore, compared to CD38 CAR-T and B2M KO T, M08+CD38&BCMA dual CAR-T cells showed better persistence in MLR assay. As shown in FIG. 6B, M08+CD38&BCMA CAR-T cells enriched during the MLR assay, the positive rate of M08+CD38&BCMA CAR-T increased to 81.5% at day 8 (before MLR, the positive rate of M08+CD38&BCMA CAR-T was 25.2%).

6.2.3. In Vitro Anti-Tumor Efficacy of CD38&BCMA CAR-T Cells

In vitro cytotoxicity assays were performed by co-culturing RPMI-8226 cells (BCMA expressed MM cell line) and various T cells, i.e., unT, BCMA CAR-T, CD38&BCMA CAR-T, CD38-4&BCMA CAR-T, M08+CD38&BCMA CAR-T, M08+CD38&BCMA CAR-T(CD38 KO), M08+CD38-I BCMA CAR-T, respectively, at Effector:Target ratio of 0.16:1 for 24 hours. Specifically. BCMA CAR-T is anti-BCMA-CD8hCD8TM4-1BBz CAR-T; CD38&BCMA CAR-T is anti-CD38-CD8hCD8TM-4-1BBz-P2A-anti-BCMA-CD8hCD8TM-4-1BBz CAR-T; and CD38-4&BCMA CAR-T is anti-CD38-CD4TM/4-1BBz P2A anti-BCMA-CD8hCD8TM-4-1BBz CAR-T. CD38-I is Isatuximab, an anti-CD38 antibody, which comprises a VH comprising an amino acid sequence of SEQ ID NO: 3, and a VL comprising an amino acid sequence of SEQ ID NO: 4.

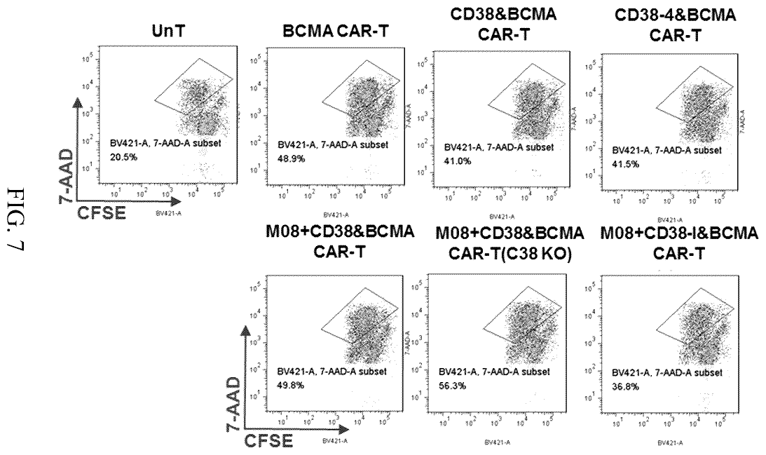

Killing of CFSE labeled target cells was examined by flow cytometry. As shown in FIG. 7, CD38&BCMA CAR-T, CD38-4&BCMA CAR-T, M08+CD38&BCMA CAR-T, M08+CD38-I&BCMA CAR-T showed similar cytotoxicity on RPMI-8226 cells as compared with BCMA CAR-T; and M08+CD38&BCMA CAR-T(CD38 KO) appeared to have higher in vitro cytotoxicity.

6.2.4. Other Exemplary Targets for NK Depletion

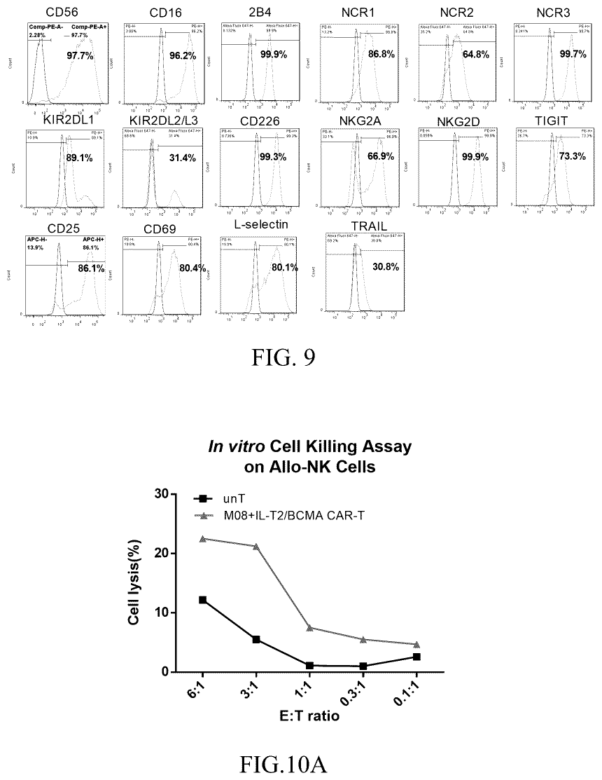

Additional NK cell targeting CARs were constructed in this study, including those targeting CD56, CD16, 2B4, NCR-1, NCR-2, NCR-3, KIR2DL1, KIR2DL2/L3, CD226, NKG2A, NKG2D, TIGIT, CD25, CD69, L-SELECTIN, TRAIL, IL-T2, CD137, KIR, for NK depletion. By FACS analysis on activated NK cells, as shown in FIG. 9, all activated NK cells tested expressed CD56, CD16, 2B4, NCR-1, NCR-2, NCR-3, CD226, NKG2D, TIGIT, CD25, CD69, and part of the activated NK cells also expressed KIR2DL1, KIR2DL2/L3, NKG2A, L-SELECTIN, TRAIL. Thus, these targets could be targets for NK depletion.

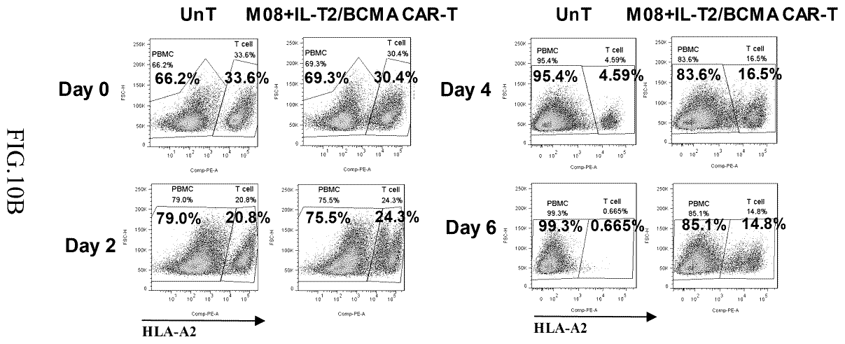

IL-T2 is an inhibitory receptor expressed by T cells, B cells, NK cells, and other immune cells. As show in FIG. 10A, in the allo-reaction assay with allo-NK cells, M08+IL-T2/BCMA CAR-T cells can deplete part of allo-NK cells. A MLR assay was then conducted with PBMCs (HLA-A2− donor) and allogeneic T cells (HLA-A2+ donor), and the ratio of PBMCs and graft T cells was 4:1. Several replicate wells were seeded for FACS staining at day 0, day 2, day 4 and day 6. At day 6, unT cells were completely rejected by allo-PBMC cells (unT cells from 33.6% at day 0 to 0.665% at day 6). Compared to unT cells, M08+IL-T2/BCMA CAR-T cell showed better survival (14.8% at day 6).

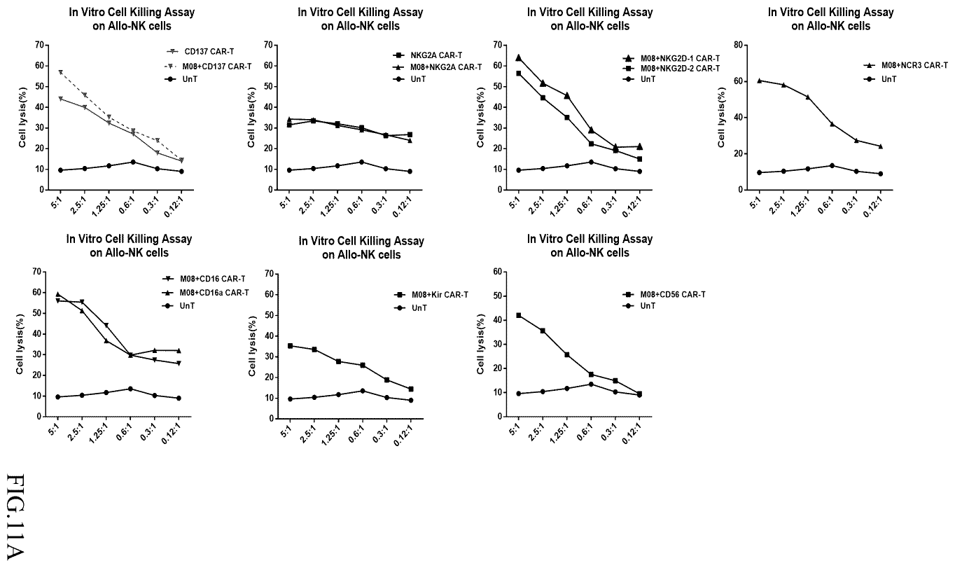

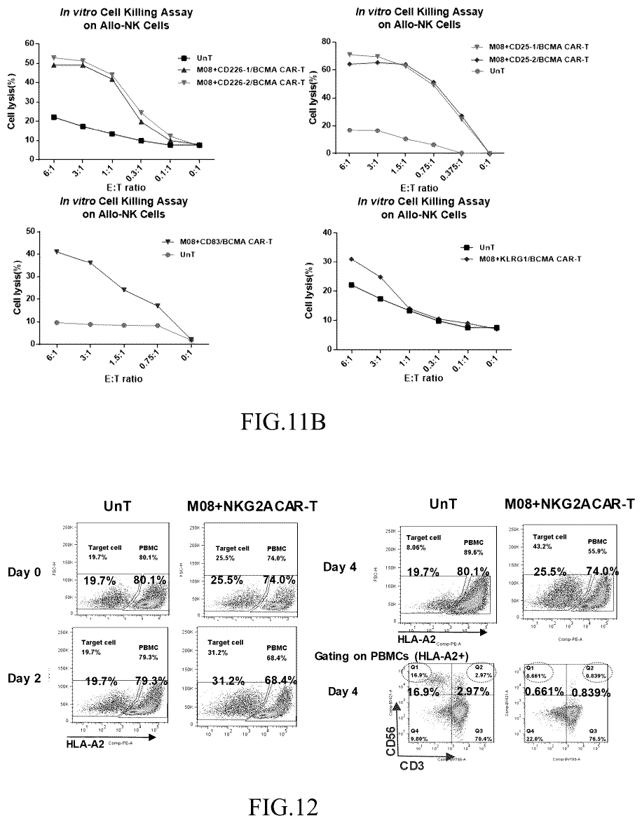

As showed in FIG. 11, CD137 CAR-T, M08+CD137 CAR-T, NKG2A CAR-T, M08+NKG2A CAR-T cells, M08+NKG2D-1 CAR-T, M08+NKG2D-2 CAR-T, M08+NCR3 CAR-T, M08+CD16 CAR-T, M08+CD16a CAR-T, M08+Kir CAR-T, M08+CD56 CAR-T (FIG. 11A), M08+CD226-1/BCMA CAR-T, M08+CD226-2/BCMA CAR-T, M08+CD25-1/BCMA CAR-T cells, M08+CD25-2/BCMA CAR-T, M08+CD83/BCMA CAR-T, M08+KLRG1/BCMA CAR-T (FIG. 11B) were seeded in the allo-reaction assay with allo-NK cells, all these CAR-T cells can deplete part of allo-NK cells. By depletion of allo-NK cells, CAR-T cells can survival well in MLR assay. As showed in FIG. 12, a MLR assay was conducted with PBMCs (HLA-A2⁺ donor) and allogeneic T cells (HLA-A2⁻ donor), and the ratio of PBMCs and graft T cells was 4:1. Several replicate wells were seeded for FACS staining at day 0, day 2 and day 4. The percentage of unT cells were decreased from 19.7% at day 0 to 8.06% at day 4. Compared to unT cells, M08+NKG2A CAR-T cell showed better survival (43.2% at day 4). When gating on allo-PBMC cells at day 4. CD56+/CD3− cells and CD56+/CD3+ cells in PBMCs were depleted by M08+NKG2A CAR-T cells.

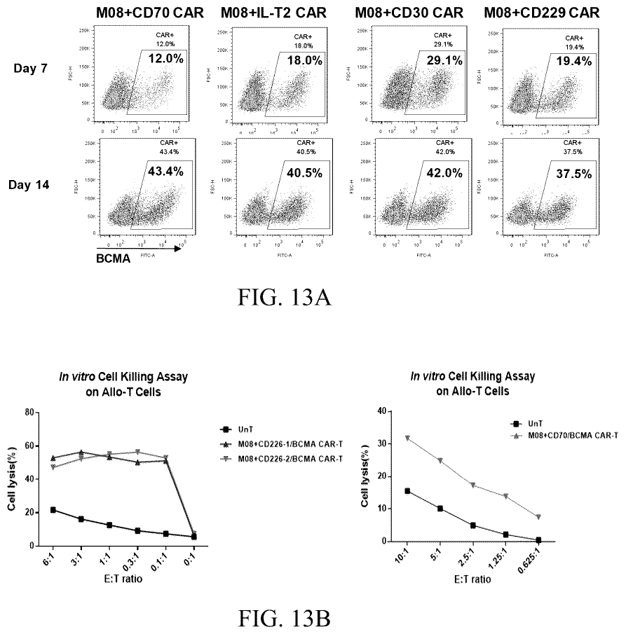

Exemplary target antigens were also expressed in activated pan-T cells. When we monitor the CAR percentage during co-culture, we found the CAR % positive cells enriched. As showed in FIG. 13A, the CAR % percentage of M08+CD70 CAR-T, M08+IL-T2 CAR-T, M08+CD30 CAR-T and M08+CD229 CAR T cells increased from day 7 and Day 14. FIG. 13B shows the anti-alloreaction effects of M08+CD226-1/BCMA CAR-T. M08+CD226-2/BCMA CAR-T, M08+CD70/BCMA CAR-T and unT cells when co-cultured with allogeneic T cells. M08+CD226-1/BCMA CAR-T, M08+CD226-2/BCMA CAR-T cells and M08+CD70/BCMA CAR-T can deplete part of allo-T cells as well.

### 6.3. Example 3—Treatment of NK Depletion Antibody Help the Survival of CAR-NK Cells

In this example, exemplary therapeutic antibodies (e.g., anti-CD38 antibodies such as Daratumumab (Johnson & Johnson)) were used to deplete host NK cells in the infusion of CAR-NK cells. Exemplary sequences in the present studies are shown in Table 2 above.

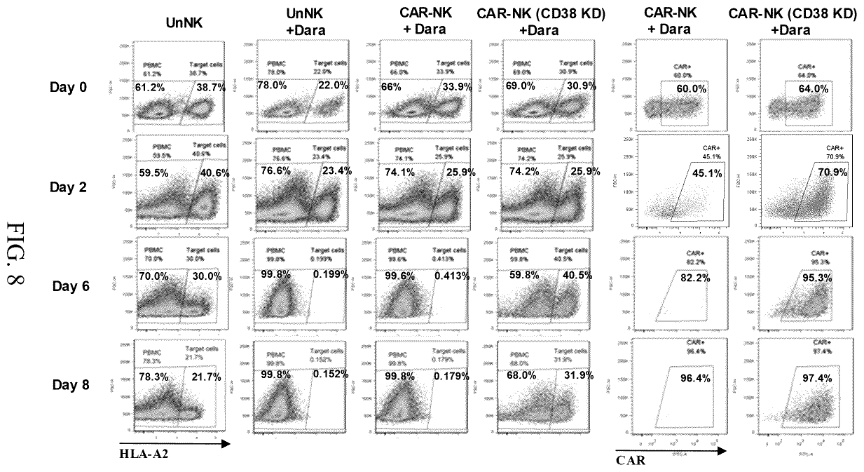

Since CD38 was also expressed on activated NK cells, to avoid sacrifice of NK cell under treatment of CD38 antibody, we knockdown CD38 on BCMA CAR-NK cells. As shown in FIG. 8, under the treatment of NK cells, both NK cells and CAR-NK cells can not survival well due to cell sacrifice, the group with BCMA CAR-NK (CD38 KD) showed good persistence with the help of CD38 antibody treatment. The CAR % cells were enriched during the treatment of CD38 antibody and allo-rejection from all genetic PBMCs (CAR % at day 0 and day 8 were 64% and 97.4% respectively).

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A method for treating a disease or disorder in a subject comprising administering to the subject an engineered immune cell, wherein the method comprises depleting the subject's immune cells, and wherein the engineered immune cell has a reduced expression of the MHC I molecule on its surface.

2. The method of claim 1, wherein the method comprises:
(i) administering to the subject an agent comprising an antibody capable of binding an antigen expressed on natural killer (NK) cells and depleting NK cells; and
(ii) administering to the subject an engineered immune cell comprising a functional exogenous receptor after step (i), wherein the functional exogenous receptor comprises an extracellular binding domain, a transmembrane domain, and an intracellular signaling domain,
wherein the engineered immune cell has reduced expression of the MHC I on cell surface,
wherein optionally the endogenous expression of the antigen targeted by the antibody is down regulated in the engineered immune cell, and
wherein optionally the antigen targeted by the antibody is also a cell surface marker on an activated T cell.

3. The method of claim 1 or 2, wherein the expression of the MHC I is reduced by expressing MHC I knockdown molecule on the engineered immune cell.

4. The method of claim 3, wherein the MHC I knockdown molecule is sICP47 protein.

5. The method of claim 4, wherein the sICP47 protein is derived from ICP47 of Simian Agent 8 (SA8), Cercopithecine herpesvirus 16 (CeHV-16), Cercopithecine herpesvirus 1 (CeHV-1), Macacine alphaherpesvirus 1, Pappine alphaherpesvirus 2 or functional variant thereof.

6. The method of claim 4 or 5, wherein the sICP47 protein comprises an amino acid sequence selected from the group consisting of SEQ ID NOs: 10-15 or variants thereof.

7. The method of any one of claims 2 to 6, wherein
(i) the antibody is capable of depleting NK cells via antibody-dependent cellular cytotoxicity (ADCC), complement-dependent cytotoxicity (CDC), antibody-dependent cellular phagocytosis (ADCP), or induced apoptosis; or
(ii) the antibody is conjugated to a drug as an antibody-drug conjugate (ADC), and the drug conjugated to the antibody is capable of depleting NK cells.

8. The method of any one of claims 2 to 7, wherein the antibody binds to an antigen selected from a group consisting of CD38, CS1, IL-T2, CD137, NKG2A, NKG2D, CD16, CD56, CD138, CD25, CD69, SLAM family members, L-SELECTIN, CD226, Kir family members, TIGIT, TRAIL, and the natural cytotoxicity receptors NCR1, NCR2, NCR3, wherein optionally SLAM family members are selected from a group consisting of SLAMF1, SLAMF4(2B4), SLAMF6, SLAMF3 and SLAMF5, and wherein optionally Kir family members are selected from a group consisting of KIR2DL1, KIR2DS1, KIR2DL2/L3, KIR2DS2, KIR2DS4, KIR3DL1, and KIR3DL2.

9. The method of any one of claims 2 to 8, wherein:
(i) the antibody is an anti-CD38 antibody, wherein optionally (i) the anti-CD38 antibody comprises a VH comprising an amino acid sequence of SEQ ID NO: 1, and/or a VL comprising an amino acid sequence of SEQ ID NO: 2; or (ii) the anti-CD38 antibody comprises a VH comprising an amino acid sequence of SEQ ID NO: 3, and/or a VL comprising an amino acid sequence of SEQ ID NO: 4;
(ii) the antibody is an anti-CS1 antibody, wherein optionally the anti-CS1 antibody comprises a VH comprising an amino acid sequence of SEQ ID NO: 5, and/or a VL comprising an amino acid sequence of SEQ ID NO: 6;
(iii) the antibody is an anti-IL-T2 antibody, wherein optionally the anti-IL-T2 antibody comprises a VH comprising an amino acid sequence of SEQ ID NO: 28, and/or a VL comprising an amino acid sequence of SEQ ID NO: 29;
(iv) the antibody is an anti-CD137 antibody, wherein optionally the anti-CD137 antibody comprises a VH comprising an amino acid sequence of SEQ ID NO: 30, and/or a VL comprising an amino acid sequence of SEQ ID NO: 31;
(v) the antibody is an anti-NKG2A antibody, wherein optionally the anti-NKG2A antibody comprises a VH comprising an amino acid sequence of SEQ ID NO: 32, and/or a VL comprising an amino acid sequence of SEQ ID NO: 33;
(vi) the antibody is an anti-NKG2D antibody, wherein optionally (i) the anti-NKG2D antibody comprises a VH comprising an amino acid sequence of SEQ ID NO: 34, and/or a VL comprising an amino acid sequence of SEQ ID NO: 35; or (ii) the anti-NKG2D antibody comprises a VH comprising an amino acid sequence of SEQ ID NO: 36, and/or a VL comprising an amino acid sequence of SEQ ID NO: 37;
(vii) the antibody is an anti-CD16 antibody, wherein optionally the anti-CD16 antibody comprises a VH comprising an amino acid sequence of SEQ ID NO: 38, and/or a VL comprising an amino acid sequence of SEQ ID NO: 39;
(viii) the antibody is an anti-CD16a antibody, wherein optionally the anti-CD16a antibody comprises a VH comprising an amino acid sequence of SEQ ID NO: 40, and/or a VL comprising an amino acid sequence of SEQ ID NO: 41;
(ix) the antibody is an anti-KIR antibody, wherein optionally the anti-KIR antibody comprises a VH comprising an amino acid sequence of SEQ ID NO: 42, and/or a VL comprising an amino acid sequence of SEQ ID NO: 43;
(x) the antibody is an anti-CD56 antibody, wherein optionally the anti-CD56 antibody comprises a VH comprising an amino acid sequence of SEQ ID NO: 44, and/or a VL comprising an amino acid sequence of SEQ ID NO: 45;
(xi) the antibody is an anti-CD226 antibody, wherein optionally (i) the anti-CD226 antibody comprises a VH comprising an amino acid sequence of SEQ ID NO: 46, and/or a VL comprising an amino acid sequence of SEQ ID NO: 47; or (ii) the anti-CD226 antibody comprises a VH comprising an amino acid sequence of SEQ ID NO: 48, and/or a VL comprising an amino acid sequence of SEQ ID NO: 49;
(xii) the antibody is an anti-CD25 antibody, wherein optionally (i) the anti-CD25 antibody comprises a VH comprising an amino acid sequence of SEQ ID NO: 50, and/or a VL comprising an amino acid sequence of SEQ ID NO: 51; or (ii) the anti-CD226 antibody comprises a VH comprising an amino acid sequence of SEQ ID NO: 52, and/or a VL comprising an amino acid sequence of SEQ ID NO: 53;
(xiii) the antibody is an anti-CD83 antibody, wherein optionally the anti-CD83 antibody comprises a VH comprising an amino acid sequence of SEQ ID NO: 54, and/or a VL comprising an amino acid sequence of SEQ ID NO: 55;
(xiv) the antibody is an anti-KLRG1 antibody, wherein optionally the anti-KLRG1 antibody comprises a VH comprising an amino acid sequence of SEQ ID NO: 56, and/or a VL comprising an amino acid sequence of SEQ ID NO: 57;
(xv) the antibody is an anti-CD70 antibody, wherein optionally the anti-CD70 antibody comprises a VH comprising an amino acid sequence of SEQ ID NO: 58, and/or a VL comprising an amino acid sequence of SEQ ID NO: 59;
(xvi) the antibody is an anti-CD30 antibody, wherein optionally the anti-CD30 antibody comprises a VH comprising an amino acid sequence of SEQ ID NO: 60, and/or a VL comprising an amino acid sequence of SEQ ID NO: 61;
(xvii) the antibody is an anti-CD229 antibody, w % herein optionally the anti-CD229 antibody comprises a VH comprising an amino acid sequence of SEQ ID NO: 62, and/or a VL comprising an amino acid sequence of SEQ ID NO: 63; or
(xviii) the antibody is an anti-NCR3 antibody, wherein optionally the anti-NCR3 antibody comprises a VH comprising an amino acid sequence of SEQ ID NO: 64, and/or a VL comprising an amino acid sequence of SEQ ID NO: 65.

10. The method of any one of claim 2 to 9, wherein the antibody is a monospecific or multispecific antibody.

11. The method of any one of claims 2 to 10, wherein the functional exogenous receptor is a T cell receptor (TCR), a chimeric antigen receptor (CAR), a chimeric TCR (cTCR), or a T cell antigen coupler (TAC)-like chimeric receptor.

12. The method of claim 11, wherein the functional exogenous receptor is a CAR.

13. The method of claim 12, wherein the extracellular binding domain of the CAR comprises an antigen binding domain capable of binding a tumor antigen.

14. The method of claim 13, wherein the tumor antigen is BCMA.

15. The method of claim 14, wherein the antigen binding domain comprises an amino acid sequence of SEQ ID NO: 7, SEQ ID NO: 8 and/or SEQ ID NO: 9.

16. An engineered immune cell comprising:
(i) a first functional exogenous receptor capable of binding and depleting natural killer (NK) cells, comprising a first extracellular binding domain capable of binding to a first antigen, a first transmembrane domain, and a first intracellular signaling domain; and
(ii) a second functional exogenous receptor comprising a second extracellular binding domain capable of binding to a second antigen, a second transmembrane domain, and a second intracellular signaling domain,
wherein the engineered immune cell has reduced expression of MHC I on cell surface,
wherein optionally the endogenous expression of the first antigen is down regulated in the engineered immune cell, and
wherein optionally the first antigen is also a cell surface marker on an activated T cell.

17. The method of claim 16, wherein the expression of MHC I is reduced by expressing MHC I knockdown molecule on the engineered immune cell.

18. The method of claim 17, wherein the MHC I knockdown molecule is sICP47 protein.

19. The method of claim 18, wherein the sICP47 protein is derived from ICP47 of Simian Agent 8 (SA8), Cercopithecine herpesvirus 16 (CeHV-16), Cercopithecine herpesvirus 1 (CeHV-1), Macacine alphaherpesvirus 1, Pappine alphaherpesvirus 2 or functional variant thereof.

20. The method of claim 18 or 19, wherein the sICP47 protein comprises an amino acid sequence selected from the group consisting of SEQ ID NOs: 10-15 or variants thereof.

21. The engineered immune cell of any one of claims 16 to 20, wherein the first functional exogenous receptor is a first chimeric antigen receptor (CAR), and wherein the second functional exogenous receptor is a T cell receptor (TCR), a chimeric TCR (cTCR), a T cell antigen coupler (TAC)-like chimeric receptor or a second CAR.

22. The engineered immune cell of any one of claims 16 to 21, wherein the first antigen is selected from a group consisting of CD38, CS1, IL-T2, CD137, NKG2A, NKG2D, CD16, CD56, CD138, CD25, CD69, SLAM family members, L-SELECTIN, CD226, Kir family members, TIGIT, TRAIL, and the natural cytotoxicity receptors NCR1, NCR2, NCR3.

23. The engineered immune cell of claim 22, wherein the SLAM family members are selected from a group consisting of SLAMF1, SLAMF4(2B4), SLAMF6, SLAMF3 and SLAMF5.

24. The engineered immune cell of claim 22, wherein the Kir family members are selected from a group consisting of KIR2DL1, KIR2DS1, KIR2DL2/L3, KIR2DS2, KIR2DS4, KIR3DL1, and KIR3DL2.

25. The engineered immune cell of any one of claims 21 to 24, wherein the first extracellular binding domain comprises:
(i) an anti-CD38 binding domain, wherein optionally (i) the anti-CD38 binding domain comprises a VH comprising an amino acid sequence of SEQ ID NO: 1, and/or a VL comprising an amino acid sequence of SEQ ID NO: 2; or (ii) the anti-CD38 binding domain comprises a VH comprising an amino acid sequence of SEQ ID NO: 3, and/or a VL comprising an amino acid sequence of SEQ ID NO: 4;
(ii) an anti-CS1 binding domain, wherein optionally the anti-CS1 binding domain comprises a VH comprising an amino acid sequence of SEQ ID NO: 5, and/or a VL comprising an amino acid sequence of SEQ ID NO: 6;
(iii) an anti-IL-T2 binding domain, wherein optionally the anti-IL-T2 binding domain comprises a VH comprising an amino acid sequence of SEQ ID NO: 28, and/or a VL comprising an amino acid sequence of SEQ ID NO: 29;
(iv) an anti-CD137 binding domain, wherein optionally the anti-CD137 binding domain comprises a VH comprising an amino acid sequence of SEQ ID NO: 30, and/or a VL comprising an amino acid sequence of SEQ ID NO: 31;
(v) an anti-NKG2A binding domain, wherein optionally the anti-NKG2A antibody comprises a VH comprising an amino acid sequence of SEQ ID NO: 32, and/or a VL comprising an amino acid sequence of SEQ ID NO: 33;
(vi) an anti-NKG2D binding domain, wherein optionally (i) the anti-NKG2D binding domain comprises a VH comprising an amino acid sequence of SEQ ID NO: 34, and/or a VL comprising an amino acid sequence of SEQ ID NO: 35; or (ii) the anti-NKG2D binding domain comprises a VH comprising an amino acid sequence of SEQ ID NO: 36, and/or a VL comprising an amino acid sequence of SEQ ID NO: 37;
(vii) an anti-CD16 binding domain, wherein optionally the anti-CD16 binding domain comprises a VH comprising an amino acid sequence of SEQ ID NO: 38, and/or a VL comprising an amino acid sequence of SEQ ID NO: 39;
(viii) an anti-CD16a binding domain, wherein optionally the anti-CD16a binding domain comprises a VH comprising an amino acid sequence of SEQ ID NO: 40, and/or a VL comprising an amino acid sequence of SEQ ID NO: 41;
(ix) an anti-KIR binding domain, wherein optionally the anti-KIR binding domain comprises a VH comprising an amino acid sequence of SEQ ID NO: 42, and/or a VL comprising an amino acid sequence of SEQ ID NO: 43;
(x) an anti-CD56 binding domain, wherein optionally the anti-CD56 binding domain comprises a VH comprising an amino acid sequence of SEQ ID NO: 44, and/or a VL comprising an amino acid sequence of SEQ ID NO: 45;
(xi) an anti-CD226 binding domain, wherein optionally (i) the anti-CD226 binding domain comprises a VH comprising an amino acid sequence of SEQ ID NO: 46, and/or a VL comprising an amino acid sequence of SEQ ID NO: 47; or (ii) the anti-CD226 binding domain comprises a VH comprising an amino acid sequence of SEQ ID NO: 48, and/or a VL comprising an amino acid sequence of SEQ ID NO: 49;
(xii) the antibody is an anti-CD25 binding domain, wherein optionally (i) the anti-CD25 binding domain comprises a VH comprising an amino acid sequence of SEQ ID NO: 50, and/or a VL comprising an amino acid sequence of SEQ ID NO: 51; or (ii) the anti-CD226 binding domain comprises a VH comprising an amino acid sequence of SEQ ID NO: 52, and/or a VL comprising an amino acid sequence of SEQ ID NO: 53;
(xiii) an anti-CD83 binding domain, wherein optionally the anti-CD83 binding domain comprises a VH comprising an amino acid sequence of SEQ ID NO: 54, and/or a VL comprising an amino acid sequence of SEQ ID NO: 55;
(xiv) an anti-KLRG1 binding domain, wherein optionally the anti-KLRG1 binding domain comprises a VH comprising an amino acid sequence of SEQ ID NO: 56, and/or a VL comprising an amino acid sequence of SEQ ID NO: 57;
(xv) an anti-CD70 binding domain, wherein optionally the anti-CD70 binding domain comprises a VH comprising an amino acid sequence of SEQ ID NO: 58, and/or a VL comprising an amino acid sequence of SEQ ID NO: 59;
(xvi) an anti-CD30 binding domain, wherein optionally the anti-CD30 binding domain comprises a VH comprising an amino acid sequence of SEQ ID NO: 60, and/or a VL comprising an amino acid sequence of SEQ ID NO: 61;
(xvii) an anti-CD229 binding domain, wherein optionally the anti-CD229 binding domain comprises a VH comprising an amino acid sequence of SEQ ID NO: 62, and/or a VL comprising an amino acid sequence of SEQ ID NO: 63; or
(xviii) an anti-NCR3 binding domain, wherein optionally the anti-NCR3 binding domain comprises a VH comprising an amino acid sequence of SEQ ID NO: 64, and/or a VL comprising an amino acid sequence of SEQ ID NO: 65.

26. The engineered immune cell of any one of claims 16 to 25, wherein the first extracellular binding domain comprises a monospecific binding domain.

27. The engineered immune cell of any one of claims 16 to 25, wherein the first extracellular binding domain comprises a multispecific or multivalent binding domain.

28. The engineered immune cell of any one of claims 21 to 27 wherein the second functional exogenous receptor is the second CAR and the second antigen is a tumor antigen.

29. The engineered immune cell of claim 28, wherein the tumor antigen is BCMA.

30. The engineered immune cell of claim 29, wherein the second extracellular antigen binding domain comprises an amino acid sequence of SEQ ID NO: 7, SEQ ID NO: 8 and/or SEQ ID NO: 9.

31. An engineered immune cell comprising a functional exogenous receptor comprising an extracellular binding domain, a transmembrane domain, and an intracellular signaling domain, wherein the extracellular binding domain comprises
(i) a first binding domain capable of binding a first antigen on natural killer (NK) cells, and
(ii) a second binding domain capable of binding a second antigen,
wherein the engineered immune cell has reduced expression of MHC I on cell surface;
wherein the engineered immune cells is capable of depleting NK cells;
wherein optionally the endogenous expression of the first antigen is down regulated in the engineered immune cell; and
wherein optionally the first antigen is also a cell surface marker on an activated T cell.

32. The method of claim 31, wherein the expression of MHC I is reduced by expressing MHC I knockdown molecule on the engineered immune cell.

33. The method of claim 32, wherein the MHC I knockdown molecule is sICP47 protein.

34. The method of claim 33, wherein the sICP47 protein is derived from ICP47 of Simian Agent 8 (SA8), Cercopithecine herpesvirus 16 (CeHV-16), Cercopithecine herpesvirus 1 (CeHV-1), Macacine alphaherpesvirus 1, Pappine alphaherpesvirus 2 or functional variant thereof.

35. The method of claim 33 or 34, wherein the sICP47 protein comprises an amino acid sequence selected from the group consisting of SEQ ID NOs: 10-15 or variants thereof.

36. The engineered immune cell of any one of claims 31 to 35, wherein the functional exogenous receptor is a chimeric antigen receptor (CAR).

37. The engineered immune cell of any one of claims 31 to 36, wherein the first antigen is selected from a group consisting of CD38, CS1, IL-T2, CD137, NKG2A, NKG2D, CD16, CD56, CD138, CD25, CD69, SLAM family members, L-SELECTIN, CD226, Kir family members, TIGIT, TRAIL, and the natural cytotoxicity receptors NCR1, NCR2, NCR3.

38. The engineered immune cell of claim 37, wherein the SLAM family members are selected from a group consisting of SLAMF1, SLAMF4(2B4), SLAMF6, SLAMF3 and SLAMF5.

39. The engineered immune cell of claim 37, wherein the Kir family members are selected from a group consisting of KIR2DL1, KIR2DS1, KIR2DL2/L3, KIR2DS2, KIR2DS4, KIR3DL1, and KIR3DL2.

40. The engineered immune cell of any one of claims 31 to 39, wherein the first binding domain comprises:
(i) an anti-CD38 binding domain, wherein optionally (i) the anti-CD38 binding domain comprises a VH comprising an amino acid sequence of SEQ ID NO: 1, and/or a VL comprising an amino acid sequence of SEQ ID NO: 2; or (ii) the anti-CD38 binding domain comprises a VH comprising an amino acid sequence of SEQ ID NO: 3, and/or a VL comprising an amino acid sequence of SEQ ID NO: 4;
(ii) an anti-CS1 binding domain, wherein optionally the anti-CS1 binding domain comprises a VH comprising an amino acid sequence of SEQ ID NO: 5, and/or a VL comprising an amino acid sequence of SEQ ID NO: 6;
(iii) an anti-IL-T2 binding domain, wherein optionally the anti-IL-T2 binding domain comprises a VH comprising an amino acid sequence of SEQ ID NO: 28, and/or a VL comprising an amino acid sequence of SEQ ID NO: 29;
(iv) an anti-CD137 binding domain, wherein optionally the anti-CD137 binding domain comprises a VH comprising an amino acid sequence of SEQ ID NO: 30, and/or a VL comprising an amino acid sequence of SEQ ID NO: 31;
(v) an anti-NKG2A binding domain, wherein optionally the anti-NKG2A antibody comprises a VH comprising an amino acid sequence of SEQ ID NO: 32, and/or a VL comprising an amino acid sequence of SEQ ID NO: 33;
(vi) an anti-NKG2D binding domain, wherein optionally (i) the anti-NKG2D binding domain comprises a VH comprising an amino acid sequence of SEQ ID NO: 34, and/or a VL comprising an amino acid sequence of SEQ ID NO: 35; or (ii) the anti-NKG2D binding domain comprises a VH comprising an amino acid sequence of SEQ ID NO: 36, and/or a VL comprising an amino acid sequence of SEQ ID NO: 37;
(vii) an anti-CD16 binding domain, wherein optionally the anti-CD16 binding domain comprises a VH comprising an amino acid sequence of SEQ ID NO: 38, and/or a VL comprising an amino acid sequence of SEQ ID NO: 39;
(viii) an anti-CD16a binding domain, wherein optionally the anti-CD16a binding domain comprises a VH comprising an amino acid sequence of SEQ ID NO: 40, and/or a VL comprising an amino acid sequence of SEQ ID NO: 41;
(ix) an anti-KIR binding domain, wherein optionally the anti-KIR binding domain comprises a VH comprising an amino acid sequence of SEQ ID NO: 42, and/or a VL comprising an amino acid sequence of SEQ ID NO: 43;
(x) an anti-CD56 binding domain, wherein optionally the anti-CD56 binding domain comprises a VH comprising an amino acid sequence of SEQ ID NO: 44, and/or a VL comprising an amino acid sequence of SEQ ID NO: 45;
(xi) an anti-CD226 binding domain, wherein optionally (i) the anti-CD226 binding domain comprises a VH comprising an amino acid sequence of SEQ ID NO: 46, and/or a VL comprising an amino acid sequence of SEQ ID NO: 47; or (ii) the anti-CD226 binding domain comprises a VH comprising an amino acid sequence of SEQ ID NO: 48, and/or a VL comprising an amino acid sequence of SEQ ID NO: 49;
(xii) the antibody is an anti-CD25 binding domain, wherein optionally (i) the anti-CD25 binding domain comprises a VH comprising an amino acid sequence of SEQ ID NO: 50, and/or a VL comprising an amino acid sequence of SEQ ID NO: 51; or (ii) the anti-CD226 binding domain comprises a VH comprising an amino acid sequence of SEQ ID NO: 52, and/or a VL comprising an amino acid sequence of SEQ ID NO: 53;
(xiii) an anti-CD83 binding domain, wherein optionally the anti-CD83 binding domain comprises a VH comprising an amino acid sequence of SEQ ID NO: 54, and/or a VL comprising an amino acid sequence of SEQ ID NO: 55;
(xiv) an anti-KLRG1 binding domain, wherein optionally the anti-KLRG1 binding domain comprises a VH comprising an amino acid sequence of SEQ ID NO: 56, and/or a VL comprising an amino acid sequence of SEQ ID NO: 57;
(xv) an anti-CD70 binding domain, wherein optionally the anti-CD70 binding domain comprises a VH comprising an amino acid sequence of SEQ ID NO: 58, and/or a VL comprising an amino acid sequence of SEQ ID NO: 59;
(xvi) an anti-CD30 binding domain, wherein optionally the anti-CD30 binding domain comprises a VH comprising an amino acid sequence of SEQ ID NO: 60, and/or a VL comprising an amino acid sequence of SEQ ID NO: 61;
(xvii) an anti-CD229 binding domain, wherein optionally the anti-CD229 binding domain comprises a VH comprising an amino acid sequence of SEQ ID NO: 62, and/or a VL comprising an amino acid sequence of SEQ ID NO: 63; or
(xviii) an anti-NCR3 binding domain, wherein optionally the anti-NCR3 binding domain comprises a VH comprising an amino acid sequence of SEQ ID NO: 64, and/or a VL comprising an amino acid sequence of SEQ ID NO: 65.

41. The engineered immune cell of any one of claims 31 to 41, wherein the first binding domain comprises a monospecific binding domain.

42. The engineered immune cell of any one of claim 31, wherein the first binding domain comprises a multispecific or multivalent binding domain.

43. The engineered immune cell of any one of claims 31 to 42, wherein the second antigen is a tumor antigen.

44. The engineered immune cell of claim 43, wherein the tumor antigen is BCMA.

45. The engineered immune cell of claim 44, wherein the antigen binding domain comprises an amino acid sequence of SEQ ID NO: 7, SEQ ID NO: 8 and/or SEQ ID NO: 9.

46. The engineered immune cell of any one of claims 16 to 45, wherein the engineered immune cell is a T cell, a natural killer cell, a macrophage, a peripheral blood mononuclear cell (PBMC), a monocyte, a neutrophil, or an eosinophil.

47. The engineered immune cell of claim 46, wherein the T cell is a cytotoxic T cell, a helper T cell, a natural killer T cell, or a γδT cell.

48. A pharmaceutical composition, comprising the engineered immune cell of any one of claims 16 to 47, and a pharmaceutically acceptable excipient.

49. A method for treating a disease or disorder in a subject comprising administering to the subject a therapeutically effective amount of the engineered T cell of any one of claims 16 to 47, or the pharmaceutical composition of claim 48.

50. The method of any one of claims 1 to 15 and 49, wherein the disease or disorder is cancer.

51. The method of claim 50, wherein the cancer is blood cancer.

52. The method of claim 50, wherein the cancer is solid tumor cancer.

53. The method of any one of claims 1 to 15 and 49 to 52, wherein the subject is a human subject in need thereof.


SEQUENCE LISTING (65 sequences)

SEQ ID NO: 1 - protein, 122 aa, Artificial Sequence [sequence omitted]

SEQ ID NO: 2 - protein, 107 aa, Artificial Sequence [sequence omitted]

SEQ ID NO: 3 - protein, 120 aa, Artificial Sequence [sequence omitted]

SEQ ID NO: 4 - protein, 109 aa, Artificial Sequence
  VL of Isatuximab (anti-CD38 antibody)
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRT

SEQ ID NO: 5 - protein, 120 aa, Artificial Sequence
  VH of elotuzumab (anti-CS1 antibody)
SEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 6 - protein, 107 aa, Artificial Sequence
  VL of elotuzumab (anti-CS1 antibody)
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 7 - protein, 118 aa, Artificial Sequence
  anti-BCMA1 sdAb
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 8 - protein, 117 aa, Artificial Sequence
  anti-BCMA2 sdAb
QVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 9 - protein, 119 aa, Artificial Sequence
  anti-BCMA3 sdAb
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSS

SEQ ID NO: 10 - protein, 256 aa, Artificial Sequence
  K5 (HHV8P)
MASKDVEEGVEGPICWICREEVGNEGIHPCACTGELDVVHPQCLSTWLTVSRNTACQMCRVIYRTRTQWRSRLNLWPEMERQEIFELFLLMSVVVAGLVGVALCTWTLLVILTAPAGTFSPGAVLGFLCFFGFYQIFIVFAFGGICRVSGTVRALYAANNTRVTVLPYRRPRRPTANEDNIELTVLVGPAGGTDEEPTDESSEGDVASGDKERDGSSGDEPDGGPNDRAGLRGTARTDLCAPTKKPVRKNHPKNNG

SEQ ID NO: 11 - protein, 183 aa, Artificial Sequence
  US6 (HHV-5)
MDLLIRLGFLLMCALPTPGERSSRDPKTLLSLSPRQQACVPRTKSHRPVCYNDTGDCTDADDSWKQLGEDFAHQCLQAAKKRPKTHKSRPNDRNLEGRLTCQRVRRLLPCDLDIHPSHRLLTLMNNCVCDGAVWNAFRLIERHGFFAVTLYLCCGITLLVVILALLCSITYESTGRGIRRCGS

SEQ ID NO: 12 - protein, 88 aa, Artificial Sequence
  ICP47 (HHV-1)
MSWALEMADTFLDTMRVGPRTYADVRDEINKRGREDREAARTAVHDPERPLLRSPGLLPEIAPNASLGVAHRRTGGTVTDSPRNPVTR

SEQ ID NO: 13 - protein, 78 aa, Artificial Sequence
  sICP47 of CeHV-16
MSSLYLAEVDAFLQSPRTRHRTCADLRRELDAYADEERREAAKAIAHPDRPLLAPPSAPPDRSRPAPRGTAHPPAASP

SEQ ID NO: 14 - protein, 81 aa, Artificial Sequence
  sICP47 of CeHV-1
MSSRYLAAVDDYLHHPSPRYQAHVDLRRELRAYADEERREAARAIAHPERPLLPPPATQAAPPQPSTREAAHPSAPTAASS

SEQ ID NO: 15 - protein, 78 aa, Artificial Sequence
  sICP47 of SA8
MSSLYLATVDAFLRNPHTRHRTCADLRRELDAYADEERREAAKAIAHPDRPLLAPPSAPPNHSHLAARETAPPPAATP

SEQ ID NO: 16 - protein, 5 aa, Artificial Sequence
  GS linker1
GGGGS

SEQ ID NO: 17 - protein, 10 aa, Artificial Sequence
  GS linker2
GGGGSGGGGS

SEQ ID NO: 18 - protein, 15 aa, Artificial Sequence
  GS linker3
GGGGSGGGGSGGGGS

SEQ ID NO: 19 - protein, 21 aa, Artificial Sequence
  CD8a leading peptide
MALPVTALLLPLALLLHAARP

SEQ ID NO: 20 - protein, 45 aa, Artificial Sequence
  CD8a hinge
TTTPAPRPPTPAPTIASQPLSLRPEACRPAAGGAVHTRGLDFACD

SEQ ID NO: 21 - protein, 24 aa, Artificial Sequence
  CD8a transmembrane
IYIWAPLAGTCGVLLLSLVITLYC

SEQ ID NO: 22 - protein, 25 aa, Artificial Sequence
  CD4 leading peptide
MNRGVPFRHLLLVLQLALLPAATQG

SEQ ID NO: 23 - protein, 44 aa, Artificial Sequence
  CD4 transmembrane
SGQVLLESNIKVLPTWSTPVQPMALIVLGGVAGLLLFIGLGIFF

SEQ ID NO: 24 - protein, 112 aa, Artificial Sequence
  intracellular domain of CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 42 aa, Artificial Sequence
  intracellular domain of 4-1BB
KRGRKKLLYIFKQPFMRPVQTTQEEDGCSCRFPEEEEGGCEL

SEQ ID NO: 26 - protein, 22 aa, Artificial Sequence
  CD3 epsilon leading peptide
MQSGTHWRVLGLCLLSVGVWGQ

SEQ ID NO: 27 - protein, 185 aa, Artificial Sequence
  CD3 epsilon
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 28 - protein, 117 aa, Artificial Sequence
  VH of anti-IL-T2 antibody
QVQLQQSGAELVKPGASVRLSCKASGYTFTAHTIHWVKQRSGQGLEWIGWLYPGSGSIKYNEKFKDKATLTADKSSSTVYMELSRLTSEDSAVYFCARHTNWDFDYWGQGTTLTVSS

SEQ ID NO: 29 - protein, 111 aa, Artificial Sequence
  VL of anti-IL-T2 antibody
NIVLTQSPASLAVSLGQRATISCKASQSVDYGGASYMNWYQQKPGQPPKLLIYAASNLESGIPARFSGSGSGTDLTLNIHPVEEEDAAMYYCQQSNEEPWTFGGGTKLEIK

SEQ ID NO: 30 - protein, 116 aa, Artificial Sequence
  VH of anti-CD137 antibody
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 31 - protein, 108 aa, Artificial Sequence
  VL of anti-CD137 antibody
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 32 - protein, 125 aa, Artificial Sequence
  VH of anti-NKG2A antibody
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 33 - protein, 107 aa, Artificial Sequence
  VL of anti-NKG2A antibody
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 34 - protein, 115 aa, Artificial Sequence
  VH of anti-NKG2D-1 antibody
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 35 - protein, 108 aa, Artificial Sequence
  VL of anti-NKG2D-1 antibody
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 36 - protein, 123 aa, Artificial Sequence
  VH of anti-NKG2D-2 antibody
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSS

SEQ ID NO: 37 - protein, 107 aa, Artificial Sequence
  VL of anti-NKG2D-2 antibody
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 38 - protein, 117 aa, Artificial Sequence
  VH of anti-CD16 antibody
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 39 - protein, 107 aa, Artificial Sequence
  VL of anti-CD16 antibody
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 40 - protein, 120 aa, Artificial Sequence
  VH of anti-CD16a antibody
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 41 - protein, 106 aa, Artificial Sequence
  VL of anti-CD16a antibody
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 42 - protein, 118 aa, Artificial Sequence
  VH of anti-KIR antibody
QIQLVQSGSELKKPGASVKVSCKASGYTFTTAGMQWVRQAPGQGLEWIGWINSHSGVPKYAEDFKGRFVFSLDTSVSTAYLQISSLKAEDTAVYFCARGGDEGVMDYWGQGTTVTVSS

SEQ ID NO: 43 - protein, 107 aa, Artificial Sequence
  VL of anti-KIR antibody
DIQMTQSPSFLSASVGDRVTITCKASQDVSTAVAWYQQKPGQPPKLLIYWTSTRHTGVPDRFSGSGSGTDYTLTISSLQAEDVAVYYCQQHYSTPWTFGGGTKVEIK

SEQ ID NO: 44 - protein, 118 aa, Artificial Sequence
  VH of anti-CD56 antibody
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 45 - protein, 112 aa, Artificial Sequence
  VL of anti-CD56 antibody
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 46 - protein, 121 aa, Artificial Sequence
  VH of anti-CD226-1 antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWVTTGPFAFDLWGQGTMVTVSS

SEQ ID NO: 47 - protein, 107 aa, Artificial Sequence
  VL of anti-CD226-1 antibody
DIQMTQSPSSLSASVGDRVTITCRASQSISSYLNWYQQKPGKAPKLLIYAASSLQSGVPSRFSGSGSGTDFTLTISSLQPEDFATYYCQQYYTLPFTFGGGTKVEIK

SEQ ID NO: 48 - protein, 118 aa, Artificial Sequence
  VH of anti-CD226-2 antibody
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSSGYYFDYWGQGTLVTVSS

SEQ ID NO: 49 - protein, 108 aa, Artificial Sequence
  VL of anti-CD226-2 antibody
EIVMTQSPATLSVSPGERATLSCRASQSVSSNLAWYQQKPGQAPRLLIYGASTRATGIPARFSGSGSGTEFTLTISSLQSEDFAVYYCQQYNKWPLFTFGGGTKVEIK

SEQ ID NO: 50 - protein, 116 aa, Artificial Sequence
  VH of anti-CD25-1 antibody
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 51 - protein, 107 aa, Artificial Sequence
  VL of anti-CD25-1 antibody
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKR

SEQ ID NO: 52 - protein, 115 aa, Artificial Sequence
  VH of anti-CD25-2 antibody
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSS

SEQ ID NO: 53 - protein, 107 aa, Artificial Sequence
  VL of anti-CD25-2 antibody
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIK

SEQ ID NO: 54 - protein, 120 aa, Artificial Sequence
  VH of anti-CD83 antibody
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 55 - protein, 111 aa, Artificial Sequence
  VL of anti-CD83 antibody
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 56 - protein, 119 aa, Artificial Sequence
  VH of anti-KLRG1 antibody
EVQLVQSGAEVKKPGASVKVSCKASGYTFTDHNMHWVKQATGQGLEWFGFINPNTGVTRYNQKFQGRVTLTINKAISTAYLELSSLRSEDTAVYYCARDYYGSAWFAYWGQGTLVTVSS

SEQ ID NO: 57 - protein, 109 aa, Artificial Sequence
  VL of anti-KLRG1 antibody
DIVMTQSPDSLAVSLGERATINCKSSQTLLYSSDQKNYLAWYQQKPGQPPKLLIYWASTRESGVPDRFSGSGSGTDFTLTISSLQAEDVAVYYCQQYYNYPTFGGGTKV

SEQ ID NO: 58 - protein, 118 aa, Artificial Sequence
  VH of anti-CD70 antibody
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 59 - protein, 111 aa, Artificial Sequence
  VL of anti-CD70 antibody
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 60 - protein, 160 aa, Artificial Sequence
  VH of anti-CD30 antibody
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNS

SEQ ID NO: 61 - protein, 111 aa, Artificial Sequence
  VL of anti-CD30 antibody
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 62 - protein, 109 aa, Artificial Sequence
  VH of anti-CD229 antibody
DIQMTQSPSSLSASVGDRVTITCPVASQSIGSSLHWYQQKPGKAPKFLIYDASSLESGVPSPVFSGSGSGTEFTLTISSLQPDDFATYYCQQYNSYPLTFGGGTKLEIK

SEQ ID NO: 63 - protein, 121 aa, Artificial Sequence
  VL of anti-CD229 antibody
QITLKESGPTLVKPTQTLTLTCTFSGFSLSTSGVGVGWIRQPPGKALEWLALIYWNDDKRYSPSLKSRLTIAKDTSKNQVVLTMTNMDPVDTATYYCARMGWNDPHMVDYWGQGTLVTVSS

SEQ ID NO: 64 - protein, 119 aa, Artificial Sequence
  VH of anti-NCR3 antibody
EIQLLESGGGLVQPGGSLRLSCAVSGFSITTTGYLIWNWVRQAPGKGLEWVGYIYSSGSTSYNPSLKSRFTISRDTSKNTFYLQMNSLRAEDTAVYYCARGDWHYFDYWGQGTMVTVSS

SEQ ID NO: 65 - protein, 108 aa, Artificial Sequence
  VL of anti-NCR3 antibody
DSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIK